(19) United States
(12) Patent Application Publication
(10) Pub. No.: US 2024/0164905 A1
(43) Pub. Date: May 23, 2024

(54) HEART VALVE REPAIR DEVICES AND DELIVERY DEVICES THEREFOR

(71) Applicant: EDWARDS LIFESCIENCES CORPORATION, Irvine, CA (US)

(72) Inventors: Yaron Herman, Givat Ada (IL); Gad Tennenbaum, Kiryat Ono (IL); Carmel Peleg, Pardes Hana-Karkur (IL); Sarit Avivi, Givat Shmuel (IL); Bezalel Haberman-Browns, Jerusalem (IL); Grant Matthew Stearns, Costa Mesa, CA (US); Lauren R. Freschauf, Mission Viejo, CA (US); Sergio Delgado, Irvine, CA (US); Eric Robert Dixon, Villa Park, CA (US); Lior Harush, Herzliya (IL)

(21) Appl. No.: 18/419,487

(22) Filed: Jan. 22, 2024

Related U.S. Application Data
(63) Continuation of application No. PCT/US2022/037983, filed on Jul. 22, 2022.
(60) Provisional application No. 63/307,589, filed on Feb. 7, 2022.
(60) Provisional application No. 63/225,387, filed on Jul. 23, 2021.

(51) Int. Cl. A61F 2/24

(52) U.S. Cl. CPC A61F 2/246; A61F 2/2466; A61F 2220/0008; A61F 2220/0091; A61F 2250/0098

(57) ABSTRACT

A valve repair device for repairing a native valve of a patient. The valve repair device includes a paddle, a gripping member, and an indicator. The paddle and/or the gripping member are movable to form an opening or capture region between the gripping member and the paddle. The indicator is configured to indicate whether a leaflet of the native valve is inserted into the opening or capture region between the paddle and the gripping member to at least a minimum insertion depth.

### RELATED APPLICATIONS

The present application is a continuation of Patent Cooperation Treaty Application no. PCT/US2022/037983, filed on Jul. 22, 2022, which claims the benefit of U.S. Provisional Application No. 63/225,387 filed on Jul. 23, 2021, titled “Heart Valve Repair Devices and Delivery Devices Therefor,” and the benefit of U.S. Provisional Application No. 63/307,589 filed on Feb. 7, 2022, titled “Heart Valve Repair Devices and Delivery Devices Therefor,” which are each incorporated herein by reference in their entireties for all purposes.

### BACKGROUND

The native heart valves (i.e., the aortic, pulmonary, tricuspid, and mitral valves) serve critical functions in assuring the forward flow of an adequate supply of blood through the cardiovascular system. These heart valves may be damaged, and thus rendered less effective, for example, by congenital malformations, inflammatory processes, infectious conditions, disease, etc. Such damage to the valves may result in serious cardiovascular compromise or death. Damaged valves can be surgically repaired or replaced during open heart surgery. However, open heart surgeries are highly invasive, and complications may occur. Transvascular techniques can be used to introduce and implant prosthetic devices in a manner that is much less invasive than open heart surgery. As one example, a transvascular technique useable for accessing the native mitral and aortic valves is the trans-septal technique. The trans-septal technique comprises advancing a catheter into the right atrium (e.g., inserting a catheter into the right femoral vein, up the inferior vena cava and into the right atrium). The septum is then punctured, and the catheter passed into the left atrium. A similar transvascular technique can be used to implant a prosthetic device within the tricuspid valve that begins similarly to the trans-septal technique but stops short of puncturing the septum and instead turns the delivery catheter toward the tricuspid valve in the right atrium.

A healthy heart has a generally conical shape that tapers to a lower apex. The heart is four-chambered and comprises the left atrium, right atrium, left ventricle, and right ventricle. The left and right sides of the heart are separated by a wall generally referred to as the septum. The native mitral valve of the human heart connects the left atrium to the left ventricle. The mitral valve has a very different anatomy than other native heart valves. The mitral valve includes an annulus portion, which is an annular portion of the native valve tissue surrounding the mitral valve orifice, and a pair of cusps, or leaflets, extending downward from the annulus into the left ventricle. The mitral valve annulus may form a “D”-shaped, oval, or otherwise out-of-round cross-sectional shape having major and minor axes. The anterior leaflet may be larger than the posterior leaflet, forming a generally “C”-shaped boundary between the abutting sides of the leaflets when they are closed together.

When operating properly, the anterior leaflet and the posterior leaflet function together as a one-way valve to allow blood to flow only from the left atrium to the left ventricle. The left atrium receives oxygenated blood from the pulmonary veins. When the muscles of the left atrium contract and the left ventricle dilates (also referred to as “ventricular diastole” or “diastole”), the oxygenated blood that is collected in the left atrium flows into the left ventricle. When the muscles of the left atrium relax and the muscles of the left ventricle contract (also referred to as “ventricular systole” or “systole”), the increased blood pressure in the left ventricle urges the sides of the two leaflets together, thereby closing the one-way mitral valve so that blood cannot flow back to the left atrium and is instead expelled out of the left ventricle through the aortic valve. To prevent the two leaflets from prolapsing under pressure and folding back through the mitral annulus toward the left atrium, a plurality of fibrous cords called chordae tendineae tether the leaflets to papillary muscles in the left ventricle.

Valvular regurgitation involves the valve improperly allowing some blood to flow in the wrong direction through the valve. For example, mitral regurgitation occurs when the native mitral valve fails to close properly and blood flows into the left atrium from the left ventricle during the systolic phase of heart contraction. Mitral regurgitation is one of the most common forms of valvular heart disease. Mitral regurgitation may have many different causes, such as leaflet prolapse, dysfunctional papillary muscles, stretching of the mitral valve annulus resulting from dilation of the left ventricle, more than one of these, etc. Mitral regurgitation at a central portion of the leaflets can be referred to as central jet mitral regurgitation and mitral regurgitation nearer to one commissure (i.e., location where the leaflets meet) of the leaflets can be referred to as eccentric jet mitral regurgitation. Central jet regurgitation occurs when the edges of the leaflets do not meet in the middle and thus the valve does not close, and regurgitation is present. Tricuspid regurgitation may be similar, but on the right side of the heart.

### SUMMARY

This summary is meant to provide some examples and is not intended to be limiting of the scope of the invention in any way. For example, any feature included in an example of this summary is not required by the claims, unless the claims explicitly recite the features. Also, the features, components, steps, concepts, etc. described in examples in this summary and elsewhere in this disclosure can be combined in a variety of ways. Various features and steps as described elsewhere in this disclosure can be included in the examples summarized here.

Devices for repairing and/or treating a native valve of a patient are disclosed. The devices can be valve repair devices, implantable devices, valve treatment devices, implants, etc. While sometimes described as an implantable device for illustration purposes in various examples herein, similar configurations can be used on other devices, e.g., valve repair devices, etc., that are not necessarily implanted and may be removed after treatment.

The devices can include an indicator (these can be the same as or similar to other indicators described anywhere herein) and a gripping member or clasp and (these can be the same as or similar to other gripping members, gripper arms, clasps, and clasp arms described anywhere herein). The devices can also include a paddle (the paddle can be the same as or similar to other paddles described anywhere herein). The paddle and/or the gripping member/clasp (e.g., a clasp arm of the clasp, a gripper arm, etc.) are movable to form an opening or capture region for receiving a leaflet. In some implementations, the opening or capture region is formed between the gripper member/clasp (e.g., a clasp arm of the clasp, etc.) and the paddle (e.g., a portion of the paddle, etc.). The indicator is configured to indicate whether a leaflet of the native valve is inserted into the opening or capture region to at least a minimum insertion depth or engagement depth. The minimum insertion depth or engagement depth can be preselected and/or configured to a particular depth as desired.

The indicators herein can be configured in a variety of shapes, sizes, and materials. In some implementations, the indicators can comprise an undulating shape, an S-shape, a C-shape, a U-shape, a V-shape, a hook shape, a check-mark shape, a swoosh shape, etc.

In some implementations, a valve repair device (or valve treatment device, etc.) includes a clasp and/or a clasp arm and an indicator (e.g., leaflet depth indicator, indicator arm, marker, sensor, electrode, etc.). The device can also include a paddle. The indicator can be configured as an indicator arm and/or can be configured such that is movable (e.g., through the clasp, paddle and/or another portion of the device) to indicate whether a leaflet of the native valve is inserted into the opening or capture region to at least a minimum insertion depth. The minimum insertion depth can be preselected and/or configured to a particular depth as desired.

In some implementations, the indicator can comprise an indicator arm, and the indicator arm can be coupled to the valve repair device at a first end of the indicator arm and at a second end of the indicator arm. The indicator arm can be coupled to an optional coaptation element of the valve repair device. The indicator arm can be compressible and can be configured to engage the leaflet of the native valve. The indicator arm can comprise one or more protrusions extending from the indicator arm. The clasp and the indicator arm can each comprise a marker comprising a radiopaque material. The capture region can be formed between a portion of the paddle and an arm of the clasp. The paddle can comprise an outer paddle and an inner paddle.

In some implementations, the indicator or indicator arm can be configured to pass through a channel, slot, gap, and/or opening of the clasp. In some implementations, the indicator or indicator arm can be configured to pass through a channel, slot, gap, and/or opening of the paddle. In some implementations, the indicator or indicator arm can be configured to pass through channel, slot, gap, and/or opening of a moveable arm of the clasp.

In some implementations, the clasp can optionally include a fixed arm. In some implementations, the fixed arm of the clasp can comprise a first beam, a second beam, and/or an engaging member between the first beam and the second beam.

In some implementations, an indicator marker can be attached to the indicator arm. The indicator arm can comprise a fixed end and a moving end. The fixed end of the indicator arm can be coupled to the clasp. The fixed end of the indicator arm can be coupled to a movable arm of the clasp. The moving end can comprise an indicator marker comprising a radiopaque material. The fixed end and the moving end can be disposed on a first side of a movable arm of the clasp.

In some implementations, the indicator or indicator arm includes a leaflet engaging member (e.g., an extension, protrusion, arm, edge, bump, dip, swoop, U-shaped portion, V-shaped portion, triangular-shaped portion, curved portion, circular portion, rectangular portion, etc.) between the fixed end and the moving end. The leaflet-engaging member can be configured to pass through at least one of a movable arm of the clasp and the paddle.

In some implementations, the leaflet-engaging member is disposed on a second side of a movable arm of the clasp. In some implementations, the leaflet-engaging member can comprise one or more protrusions extending from the leaflet-engaging member.

In some implementations, an indicator can comprise a first arm and a second arm. The first arm and the second arm can be coupled with the moving end and can be connected at a connection point at the fixed end.

In some implementations, the indicator arm is formed from a portion of the clasp. The indicator arm can be formed between outer beams of a movable arm of the clasp and/or outside outer beams of the clasp (or clasp arm of the clasp).

In some implementations, the indicator arm can comprise a twisted portion. The twisted portion can comprise one or more twists between 0 degrees and 180 degrees.

In some implementations, the indicator arm can comprise a first arm portion and a second arm portion. At least one of the first arm portion and a second arm portion can be formed between outer beams of the clasp and/or outside outer beams of the clasp. At least one of the first arm portion and a second arm portion can be formed from a portion of first beam of the clasp. In some implementations, the first arm portion can comprise a twisted portion. The twisted portion of the first arm portion can comprise one or more twists between 0 degrees and 180 degrees clockwise.

In some implementations, a second arm portion can comprise a twisted portion. The twisted portion of the second arm portion can comprise one or more twists between 0 degrees and 180 degrees counterclockwise.

In some implementations, the first arm portion and the second arm portion are coupled with the moving end at a connection point. The connection point can comprise an indicator marker comprising a radiopaque material press fit into at least one of the first arm portion and the second arm portion.

In some implementations, a valve repair system for repairing a native valve of a patient includes a delivery system and a valve repair device coupled to the delivery system. The valve repair device can include a paddle, an indicator (e.g., leaflet depth indicator, indicator arm, sensor, etc.), and a gripping member or clasp. The gripping member/clasp and/or the paddle can be movable to form an opening or capture region to receive a leaflet of the native valve. The indicator is coupled to the valve repair device. In some implementations, the indicator is configured as an indicator arm and/or is movable to indicate whether the leaflet of the native valve is inserted into the opening or capture region to at least a minimum insertion depth. The device can be configured to have different minimum insertion depths as desired (e.g., a minimum depth of one or more of 3 mm, 4 mm, 5 mm, 6 mm, 7 mm, 8 mm, etc.). The indicator or indicator arm can be configured to pass through one or more of the paddle and the gripping member/clasp.

In some implementations, a valve repair device includes a gripping member or clasp (e.g., a clasp arm, gripper arm, etc.) and a leaflet depth indicator. The leaflet depth indicator includes at least a first electrode and a second electrode. The first electrode and the second electrode provide electrical signals to indicate whether a leaflet of the native valve is inserted into an opening or capture region to at least a minimum insertion depth. The minimum insertion depth can be preselected and/or configured to a particular depth as desired. The device can also include a paddle.

In some implementations, a valve repair device for repairing a native heart valve includes a gripping member or clasp (e.g., a clasp arm, gripper arm, etc.) and a leaflet depth indicator. The clasp (or a clasp arm/gripper arm of the clasp) can be movable to form an opening or capture region for receiving a native leaflet of the native valve. The leaflet depth indicator can comprise a first electrode and a second electrode. The first electrode and the second electrode can provide electrical signals to indicate whether a leaflet of the native valve is inserted into the opening to a particular insertion depth.

In some implementations, the electrical signals comprises an intracardiac electrocardiogram signal or a bioimpedance signal. The first electrode and the second electrode can be coupled to the gripping member/clasp (or clasp arm, gripper arm, etc.). In some implementations, the gripping member/clasp comprises a movable arm, and the first electrode and the second electrode are coupled to the movable arm. In some implementations, the first electrode and the second electrode are coupled to an indicator arm. The indicator arm can be coupled to the valve repair device and is movable in an opening or capture region.

In some implementations, a valve repair system for repairing a native heart valve includes a delivery system and valve repair device. The valve repair device is releasably coupled to the delivery system. The valve repair device includes a gripping member or clasp (e.g., a clasp arm, gripper arm, etc.) and a leaflet depth indicator. The gripping member/clasp (e.g., a portion, clasp arm, gripper arm, etc. thereof) is movable to form an opening or capture region for receiving a native leaflet of the native valve. The leaflet depth indicator comprises a first electrode and a second electrode. The first electrode and the second electrode provide electrical signals to indicate whether a leaflet of the native valve is inserted into the opening or capture region to at least a minimum insertion depth. The minimum insertion depth can be preselected and/or configured to a particular depth as desired.

In some implementations, a leaflet depth indicator can be integrally formed with the gripping member/clasp. For example, the leaflet depth indicator can be formed from the same material as the gripping member/clasp. In some implementations, the gripping member/clasp and the leaflet depth indicator can be cut from a single piece of sheet material.

In some implementations, the material of the leaflet depth indicator can be bent, twisted and/or shape set relative to the material of the gripping member/clasp such that the leaflet depth indicator is positioned in a plane such that it can contact a native leaflet and determine whether the gripping member/clasp has properly engaged the native leaflet. The leaflet depth indicator can extend from a movable arm of the clasp, aa hinge portion of the clasp, and/or a fixed arm of the clasp.

In some implementations, a valve repair device includes a gripping member or clasp (and/or clasp arm, gripper arm, etc. thereof) and an indicator. The device can also include a paddle. The valve repair device can also include an insulator disposed between at least a portion of the gripping member/clasp/clasp arm and the indicator. The indicator includes one or more electrically conductive indicator contacts which can connect to a sensor to indicate whether a leaflet of the native valve is inserted into the opening or capture region to at least a minimum insertion depth. The minimum insertion depth can be preselected and/or configured to a particular depth as desired.

In some implementations, the signal can be passed to the sensors by electrical wiring from the valve repair device to the sensors. The signal can be passed to the sensors by electrical conductance from the indicator through a portion of the valve repair device. In some implementations, the signal is passed to the sensors by electrical conductance from the indicator through at least one of an electrically conductive fixed arm, an electrically conductive coaptation element, an electrically conductive collar, an electrically conductive catheter coupler, and an electrically conductive actuation line

In some implementations, a gripping member or clasp can include movable arm and a fixed arm, as well as a first indicator plate coupled to the fixed arm, and a second indicator plate coupled to the movable arm.

In some implementations the valve repair device can include a bar coupled to the clasp wherein the bar includes a leaflet engaging portion and a device engaging portion. The leaflet engaging portion can reinforce the paddle and can prevent or inhibit a leaflet from bunching around the indicator or between portions of the indicator.

In some implementations, a valve repair device for repairing a native valve of a patient includes a gripping member or clasp (and/or clasp arm, gripper arm, etc.) and an indicator. The gripping member/clasp (or a portion, arm, etc. of the gripping member/clasp) is movable to form an opening or capture region for capturing a leaflet of the native valve. The indicator is coupled to the valve repair device. The indicator can comprise one or more electrically conductive indicator contacts. The indicator can indicate whether the leaflet of the native valve is inserted into the opening or capture region to at least a minimum insertion depth.

In some implementations, the indicator can comprise two electrically conductive indicator contacts. The two electrically conductive indicator contacts can be bridged when the gripping member/clasp is in a closed position and leaflet tissue is not inserted to the minimum insertion depth. Or the two electrically conductive indicator contacts can be electrically isolated when the gripping member/clasp is in a closed position and leaflet tissue is inserted to the minimum insertion depth. The one or more electrically conductive indicator contacts can be disposed on a paddle of the valve repair device.

In some implementations, a valve repair device for repairing a native heart valve includes an electrically conductive clasp (or other gripping member), an electrically conductive paddle, and an insulator. The insulator is disposed between a portion of the electrically conductive clasp and the electrically conductive paddle. The electrically conductive clasp is configured to move to form a capture region for capturing a leaflet of the native valve. The electrically conductive clasp contacts the electrically conductive paddle when the clasp is in a closed position and leaflet tissue is not inserted to a minimum insertion depth.

In some implementations, the clasp is electrically isolated from the electrically conductive paddle when the clasp is in a closed position and leaflet tissue is inserted to the minimum insertion depth.

In some implementations, the electrically conductive paddle is coupled to an electrically conductive collar. The electrically conductive paddle can be coupled to the electrically conductive collar by an electrically conductive coaptation element.

In some implementations, a valve repair system includes a valve repair device and a delivery device. The valve repair device includes an electrically conductive clasp (or other gripping member), an electrically conductive paddle, an insulator, and an electrically conductive collar. The insulator is disposed between a portion of the electrically conductive clasp and the electrically conductive paddle. The electrically conductive collar is electrically coupled to the electrically conductive paddle. The delivery device includes a catheter, and electrically conductive coupler, and an electrically conductive actuation line. The electrically conductive coupler is releasably coupled to the electrically conductive collar. The electrically conductive actuation line is connected to the electrically conductive clasp configured to move the clasp to form a capture region for capturing a leaflet of the native valve. The electrically conductive clasp contacts the electrically conductive paddle when the clasp is in a closed position and leaflet tissue is not inserted to a minimum insertion depth.

In some implementations, the electrically conductive paddle is coupled to the electrically conductive collar by an electrically conductive coaptation element. The clasp can be electrically isolated from the electrically conductive paddle when the clasp is in a closed position and leaflet tissue is inserted to the minimum insertion depth.

In some implementations, a valve repair device for repairing a native heart valve includes an electrically conductive clasp (or other gripping member), an electrically conductive leaflet depth indicator, and an insulator. The insulator is disposed between a portion of the electrically conductive clasp and the electrically conductive leaflet depth indicator. The electrically conductive clasp (or clasp arm) is configured to move to form a capture region for capturing a leaflet of the native valve.

In some implementations, the electrically conductive leaflet depth indicator contacts the electrically conductive clasp when the clasp is in a closed position and leaflet tissue is not inserted to a minimum insertion depth.

In some implementations, the clasp is electrically isolated from the electrically conductive leaflet depth indicator when the clasp is in a closed position and leaflet tissue is inserted to the minimum insertion depth.

In some implementations, the electrically conductive leaflet depth indicator moves relative to the clasp (or clasp arm) when the clasp is in a closed position and leaflet tissue is inserted to the minimum insertion depth.

In some implementations, a valve repair device includes a clasp (or clasp arm), an indicator, and a sensor. The clasp includes a movable arm and a fixed arm. The clasp (or movable arm thereof) is movable to form an opening or capture region for capturing a leaflet of the native valve. The indicator comprises a first indicator plate coupled to the fixed arm and a second indicator plate coupled to the movable arm. The indicator is configured to detect one or more electrical characteristics of blood or tissue. The sensor is coupled to the indicator.

In some implementations, the sensor is configured to measure one or more of resistance, inductance, capacitance, voltage, current, and impedance. The sensor can be configured to measure impedance. The sensor can be configured to compare the sensed one or more electrical characteristics to previously measured electrical characteristics that correspond to known tissue and blood samples. The sensor can be configured to determine whether tissue is engaged. The sensor is configured to differentiate between leaflet tissue and chordae tendinea tissue.

In some implementations a first impedance value is measured in a method of identifying a clasp condition (or gripper member condition). The first impedance value is compared to previously measured impedance values. One or more of the condition or location of the clasp based is determined or estimated based on the comparison. The method(s) can be performed on a living animal or on a simulation, such as on a cadaver, cadaver heart, simulator (e.g., with the body parts, heart, leaflet, tissue, etc. being simulated), etc.

In some implementations, a valve repair device for repairing a native valve of a patient includes a paddle, an indicator, a bar, and a gripping member or clasp. The gripping member/clasp (or a portion or movable arm thereof) is movable to form a capture region for capturing a leaflet of the native valve. In some implementations, the indicator is coupled to the gripping member/clasp. The indicator can be configured as an indicator arm and/or configured to be movable to indicate whether the leaflet of the native valve is inserted into the opening or capture region to at least a minimum insertion depth. The minimum insertion depth can be preselected and/or configured to a particular depth as desired. The bar is coupled to the paddle. The bar reinforces the paddle and reduces a space in the capture region.

In some implementation. The bar can comprise a leaflet engaging portion and a device engaging portion. The leaflet engaging portion can comprise a one or more crests positioned to make contact with the leaflet. The crest can overlap the indicator when viewed from the side.

A further understanding of the nature and advantages of the present invention are set forth in the following description and claims, particularly when considered in conjunction with the accompanying drawings in which like parts bear like reference numerals.

### BRIEF DESCRIPTION OF THE DRAWINGS

To further clarify various aspects of examples in the present disclosure, a more particular description of certain examples and implementations will be made by reference to various aspects of the appended drawings. It is appreciated that these drawings depict only example implementations of the present disclosure and are therefore not to be considered limiting of the scope of the disclosure. Moreover, while the figures can be drawn to scale for some examples, the figures are not necessarily drawn to scale for all examples. Examples and other features and advantages of the present disclosure will be described and explained with additional specificity and detail through the use of the accompanying drawings in which:

FIG. 1 illustrates a cutaway view of the human heart in a diastolic phase;

FIG. 2 illustrates a cutaway view of the human heart in a systolic phase;

FIG. 3 illustrates a cutaway view of the human heart in a systolic phase showing valve regurgitation;

FIG. 4 is the cutaway view of FIG. 3 annotated to illustrate a natural shape of mitral valve leaflets in the systolic phase;

FIG. 5 illustrates a healthy mitral valve with the leaflets closed as viewed from an atrial side of the mitral valve;

FIG. 6 illustrates a dysfunctional mitral valve with a visible gap between the leaflets as viewed from an atrial side of the mitral valve;

FIG. 7 illustrates a tricuspid valve viewed from an atrial side of the tricuspid valve;

FIGS. 8-14 show an example of an implantable device or implant, in various stages of deployment;

FIG. 15 shows an example of an implantable device or implant that is similar to the device illustrated by FIGS. 8-14, but where the paddles are independently controllable;

FIGS. 16-21 show the example implantable device or implant of FIGS. 8-14 being delivered and implanted within a native valve;

FIG. 22 shows a perspective view of an example implantable device or implant in a closed position;

FIG. 23 shows a front view of the implantable device or implant of FIG. 22;

FIG. 24 shows a side view of the implantable device or implant of FIG. 22;

FIG. 25 shows a front view of the implantable device or implant of FIG. 22 with a cover covering the paddles and a coaptation element or spacer;

FIG. 26 shows a top perspective view of the implantable device or implant of FIG. 22 in an open position;

FIG. 27 shows a bottom perspective view of the implantable device or implant of FIG. 22 in an open position;

FIG. 28 shows a clasp for use in an implantable device or implant;

FIG. 29 shows a portion of native valve tissue grasped by a clasp;

FIG. 30 shows a side view of an example implantable device or implant in a partially-open position with clasps in a closed position;

FIG. 31 shows a side view of an example implantable device or implant in a partially-open position with clasps in an open position;

FIG. 32 shows a side view of an example implantable device or implant in a half-open position with clasps in a closed position;

FIG. 33 shows a side view of an example implantable device or implant in a half-open position with clasps in an open position;

FIG. 34 shows a side view of an example implantable device or implant in a three-quarters-open position with clasps in a closed position;

FIG. 35 shows a side view of an example implantable device or implant in a three-quarters-open position with clasps in an open position;

FIG. 36 shows a side view of an example implantable device in a fully open or full bailout position with clasps in a closed position;

FIG. 37 shows a side view of an example implantable device in a fully open or full bailout position with clasps in an open position;

FIGS. 38-49 show the example implantable device or implant of FIGS. 30-38, including a cover, being delivered and implanted within a native valve;

FIG. 50 shows a schematic view illustrating a path of native valve leaflets along each side of a coaptation element or spacer of an example valve repair device or implant;

FIG. 51 shows a top schematic view illustrating a path of native valve leaflets around a coaptation element or spacer of an example valve repair device or implant;

FIG. 52 shows a coaptation element or spacer in a gap of a native valve as viewed from an atrial side of the native valve;

FIG. 53 shows a valve repair device or implant attached to native valve leaflets with the coaptation element or spacer in the gap of the native valve as viewed from a ventricular side of the native valve;

FIG. 54 shows a perspective view of a valve repair device or implant attached to native valve leaflets with the coaptation element or spacer in the gap of the native valve shown from a ventricular side of the native valve;

FIG. 55 shows a perspective view of an example implantable device or implant in a closed position;

FIG. 56 shows a perspective view of an example clasp of an example implantable device or implant in a closed position;

FIG. 57 illustrates a valve repair device with paddles in an open position;

FIG. 58 illustrates the valve repair device of FIG. 57, in which the paddles are in the open position and gripping members (e.g., gripping arms, clasp arms, etc.) are moved to create a wider gap between the gripping members and paddles;

FIG. 59 illustrates the valve repair device of FIG. 57, in which the valve repair device is in the position shown in FIG. 57 with valve tissue placed between the gripping members and the paddles;

FIG. 60 illustrates the valve repair device of FIG. 57, in which the gripping members are moved to lessen the gap between the gripping members and the paddles;

FIGS. 61A-61B illustrate the movement of the paddles of the valve repair device of FIG. 57 from the open position to a closed position;

FIG. 62 illustrates the valve repair device of FIG. 57 in a closed position, in which the gripping members are engaging valve tissue;

FIG. 63 illustrates the valve repair device of FIG. 57 after being disconnected from a delivery device and attached to valve tissue, in which the valve repair device is in a closed and locked condition;

FIGS. 64-67 show an example clasp or leaflet capture portion being deployed to engage with a leaflet of a native valve;

FIGS. 68-77 show a device having clasps with indicator arms being delivered and deployed within a native valve;

FIGS. 78-84 illustrate an example valve repair device with paddles in an open position;

FIGS. 85-87 show a device having clasps with indicator arms;

FIGS. 88-93 illustrate an example of a clasp having an indicator arm with a shaped end;

FIGS. 94 and 95A-95G illustrate an example of a clasp having an indicator arm with a shaped portion in a closed position;

FIGS. 96A and 96B illustrate the clasp having an indicator arm with a shaped portion of FIG. 94 in an open position;

FIGS. 97-98 illustrate an example valve repair device having clasps with leaflet depth indicators;

FIGS. 99-101 illustrate example clasps having leaflet depth indicators;

FIGS. 102A and 102B illustrate a valve repair device having clasps with leaflet depth indicators;

FIGS. 103-109 illustrate example clasps having leaflet depth indicators;

FIG. 110 illustrates a fixed end of a leaflet depth indicator;

FIGS. 111-114 illustrate example clasps having leaflet depth indicators;

FIGS. 115-116 illustrate a device having clasps with leaflet depth indicators;

FIGS. 117-118 illustrate example leaflet paths between the clasps and the leaflet depth indicators;

FIGS. 119-120 illustrate example leaflet depth indicators for clasps and/or capture devices;

FIGS. 121-126 illustrate example clasps having leaflet depth indicators;

FIGS. 127-128 illustrate an example implantable device with clasps having leaflet depth indicators;

FIG. 129 illustrates an example clasp having a leaflet depth indicator;

FIG. 130 illustrates an example device with clasps having leaflet depth indicators;

FIG. 131 illustrates an example clasp having a leaflet depth indicator;

FIG. 132 illustrates an example implantable device with clasps having leaflet depth indicators;

FIG. 133 illustrates an example clasp having a leaflet depth indicator;

FIG. 134 illustrates an example implantable device with clasps having leaflet depth indicators;

FIG. 135 illustrates an example clasp having a leaflet depth indicator;

FIGS. 136, 137A, and 137B illustrate intracardiac electrocardiogram (IECG) signals measured by electrodes of example leaflet depth indicators;

FIGS. 137C-137F illustrate bipolar IECG signals measured from electrodes of example leaflet depth indicators;

FIG. 138 illustrates an example clasp having a leaflet depth indicator;

FIG. 139 illustrates an example clasp having an integral leaflet depth indicator;

FIG. 140A illustrates an example clasp having arms that can be formed into an integral leaflet depth indicator;

FIG. 140B illustrates an example clasp having an integral leaflet depth indicator made from the arms shown in FIG. 140A;

FIG. 140C illustrates an example clasp having an integral leaflet depth indicator made from the arms shown in FIG. 140A;

FIG. 141A illustrates an example clasp having arms that can be formed into a movable arm of the clasp and arms that can be formed into an integral leaflet depth indicator;

FIG. 141B illustrates an example clasp having arms that can be formed into a movable arm of the clasp and arms that can be formed into an integral leaflet depth indicator;

FIG. 141C illustrates an example clasp having a movable arm and integral leaflet depth indicator made from the arms shown in FIG. 141A or FIG. 141B;

FIG. 141D illustrates an example clasp having arms that can be formed into a movable arm of the clasp and arms that can be formed into an integral leaflet depth indicator;

FIG. 142A illustrates an example clasp having an integral leaflet depth indicator where a valve leaflet is not inserted to a depth that causes displacement of the leaflet depth indicator;

FIG. 142B illustrates an example clasp having an integral leaflet depth indicator where a valve leaflet is inserted to a depth that causes displacement of the leaflet depth indicator;

FIG. 143A illustrates an example clasp having an integral leaflet depth indicator where a valve leaflet is not inserted to a depth that causes displacement of the leaflet depth indicator;

FIG. 143B illustrates an example clasp having an integral leaflet depth indicator where a valve leaflet is inserted to a depth that causes displacement of the leaflet depth indicator;

FIGS. 144-147 illustrate example devices with clasps having an electrical leaflet depth indicator;

FIGS. 148-155 illustrate example clasps with leaflet depth indicators that are configured to visually and electrically indicate leaflet insertion;

FIGS. 156, 156A, 156B, 156C, and 156D illustrate clasps having different sensing plate configurations;

FIGS. 157-158 illustrate example clasps having electrical leaflet depth indicators;

FIG. 159 illustrates an example clasp having an electrical leaflet depth indicator of one of FIGS. 157-158 sensing blood;

FIG. 160 illustrates an example clasp having an electrical leaflet depth indicator of one of FIGS. 157-158 sensing a valve leaflet;

FIG. 161 illustrates an example clasp having an electrical leaflet depth indicator of one of FIGS. 157-158 sensing chordae tendineae;

FIG. 162 illustrates a circuit used to measure impedance in accordance with some implementations of clasps with electrical indicators;

FIG. 163 illustrates examples of calculations of components of impedance;

FIG. 164 illustrates an implementation of a method of identifying a clasp condition based on an electrical measurement;

FIG. 165-169 illustrate example devices and/or portions of devices with clasps having a leaflet depth indicator.

### DETAILED DESCRIPTION

The following description refers to the accompanying drawings, which illustrate example implementations of the present disclosure. Other implementations having different structures and operation do not depart from the scope of the present disclosure.

Example implementations of the present disclosure are directed to systems, devices, methods, etc. for repairing a defective heart valve. For example, various implementations of valve repair devices, implantable devices, implants, and systems (including systems for delivery thereof) are disclosed herein, and any combination of these options can be made unless specifically excluded. In other words, individual components of the disclosed devices and systems can be combined unless mutually exclusive or otherwise physically impossible. Further, the techniques and methods herein can be performed on a living animal or on a simulation, such as on a cadaver, cadaver heart, simulator (e.g., with the body parts, heart, tissue, etc. being simulated), etc.

As described herein, when one or more components are described as being connected, joined, affixed, coupled, attached, or otherwise interconnected, such interconnection can be direct as between the components or can be indirect such as through the use of one or more intermediary components. Also as described herein, reference to a “member,” “component,” or “portion” shall not be limited to a single structural member, component, or element but can include an assembly of components, members, or elements. Also as described herein, the terms “substantially” and “about” are defined as at least close to (and includes) a given value or state (preferably within 10% of, more preferably within 1% of, and most preferably within 0.1% of). The terms “clasp” and “clasp arm” are often used herein with respect to specific examples, but the terms “gripping member” and/or “gripper arm” can be used in place of and function in the same or similar ways, even if not configured in the same way as a typical clasp.

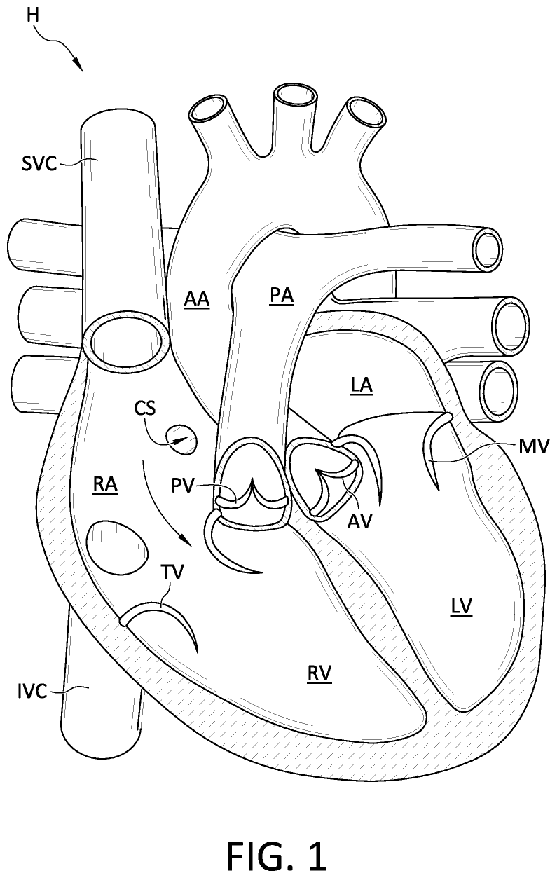

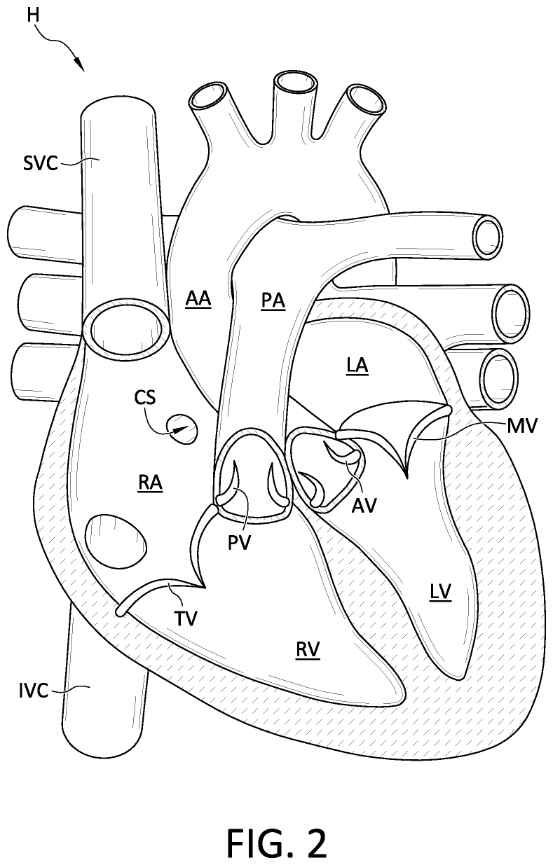

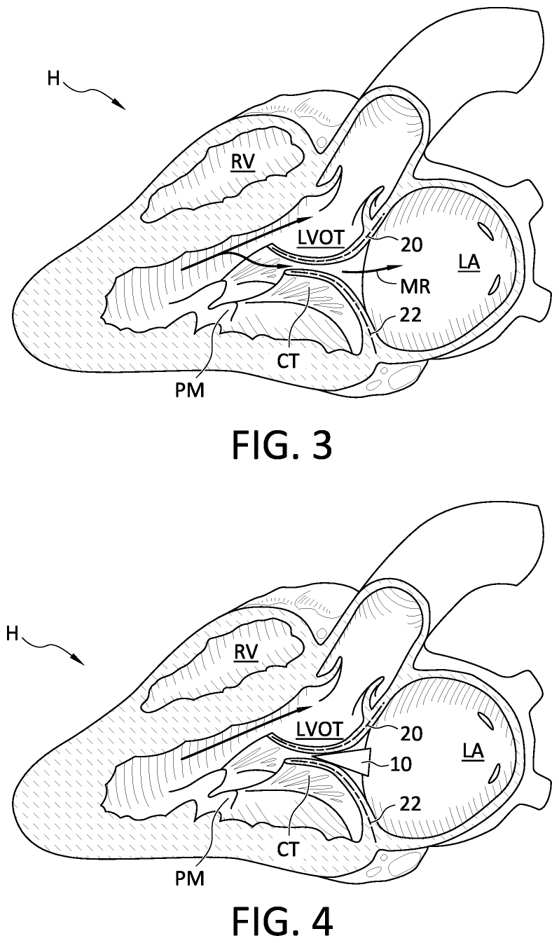

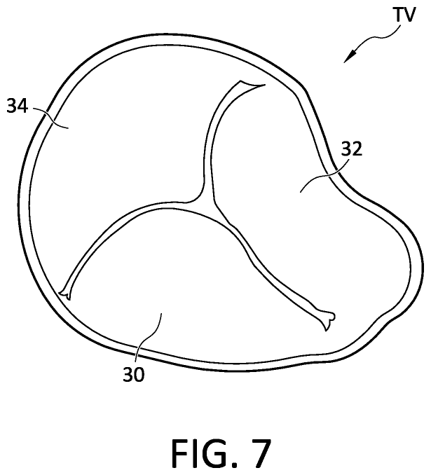

FIGS. 1 and 2 are cutaway views of the human heart H in diastolic and systolic phases, respectively. The right ventricle RV and left ventricle LV are separated from the right atrium RA and left atrium LA, respectively, by the tricuspid valve TV and mitral valve MV; i.e., the atrioventricular valves. Additionally, the aortic valve AV separates the left ventricle LV from the ascending aorta AA, and the pulmonary valve PV separates the right ventricle from the pulmonary artery PA. Each of these valves has flexible leaflets (e.g., leaflets 20, 22 shown in FIGS. 3-6 and leaflets 30, 32, 34 shown in FIG. 7) extending inward across the respective orifices that come together or “coapt” in the flow stream to form the one-way, fluid-occluding surfaces. The native valve repair systems of the present application are frequently described and/or illustrated with respect to the mitral valve MV. Therefore, anatomical structures of the left atrium LA and left ventricle LV will be explained in greater detail. However, the devices described herein can also be used in repairing other native valves, e.g., the devices can be used in repairing the tricuspid valve TV, the aortic valve AV, and the pulmonary valve PV.

The left atrium LA receives oxygenated blood from the lungs. During the diastolic phase, or diastole, seen in FIG. 1, the blood that was previously collected in the left atrium LA (during the systolic phase) moves through the mitral valve MV and into the left ventricle LV by expansion of the left ventricle LV. In the systolic phase, or systole, seen in FIG. 2, the left ventricle LV contracts to force the blood through the aortic valve AV and ascending aorta AA into the body. During systole, the leaflets of the mitral valve MV close to prevent the blood from regurgitating from the left ventricle LV and back into the left atrium LA and blood is collected in the left atrium from the pulmonary vein. In some implementations, the devices described by the present application are used to repair the function of a defective mitral valve MV. That is, the devices are configured to help close the leaflets of the mitral valve to prevent or inhibit blood from regurgitating from the left ventricle LV and back into the left atrium LA. Many of the devices described in the present application are designed to easily grasp and secure the native leaflets around a coaptation element or spacer that beneficially acts as a filler in the regurgitant orifice to prevent or inhibit back flow or regurgitation during systole, though this is not necessary.

Referring now to FIGS. 1-7, the mitral valve MV includes two leaflets, the anterior leaflet 20 and the posterior leaflet 22. The mitral valve MV also includes an annulus 24, which is a variably dense fibrous ring of tissues that encircles the leaflets 20, 22. Referring to FIGS. 3 and 4, the mitral valve MV is anchored to the wall of the left ventricle LV by chordae tendineae CT. The chordae tendineae CT are cord-like tendons that connect the papillary muscles PM (i.e., the muscles located at the base of the chordae tendineae CT and within the walls of the left ventricle LV) to the leaflets 20, 22 of the mitral valve MV. The papillary muscles PM serve to limit the movements of leaflets 20, 22 of the mitral valve MV and prevent the mitral valve MV from being reverted. The mitral valve MV opens and closes in response to pressure changes in the left atrium LA and the left ventricle LV. The papillary muscles PM do not open or close the mitral valve MV. Rather, the papillary muscles PM support or brace the leaflets 20, 22 against the high pressure needed to circulate blood throughout the body. Together the papillary muscles PM and the chordae tendineae CT are known as the subvalvular apparatus, which functions to keep the mitral valve MV from prolapsing into the left atrium LA when the mitral valve closes. As seen from a Left Ventricular Outflow Tract (LVOT) view shown in FIG. 3, the anatomy of the leaflets 20, 22 is such that the inner sides of the leaflets coapt at the free end portions and the leaflets 20, 22 start receding or spreading apart from each other. The leaflets 20, 22 spread apart in the atrial direction, until each leaflet meets with the mitral annulus.

Various disease processes can impair proper function of one or more of the native valves of the heart H. These disease processes include degenerative processes (e.g., Barlow's Disease, fibroelastic deficiency, etc.), inflammatory processes (e.g., Rheumatic Heart Disease), and infectious processes (e.g., endocarditis, etc.). In addition, damage to the left ventricle LV or the right ventricle RV from prior heart attacks (i.e., myocardial infarction secondary to coronary artery disease) or other heart diseases (e.g., cardiomyopathy, etc.) can distort a native valve's geometry, which can cause the native valve to dysfunction. However, the majority of patients undergoing valve surgery, such as surgery to the mitral valve MV, suffer from a degenerative disease that causes a malfunction in a leaflet (e.g., leaflets 20, 22) of a native valve (e.g., the mitral valve MV), which results in prolapse and regurgitation.

Generally, a native valve may malfunction in different ways: including (1) valve stenosis; and (2) valve regurgitation. Valve stenosis occurs when a native valve does not open completely and thereby causes an obstruction of blood flow. Typically, valve stenosis results from buildup of calcified material on the leaflets of a valve, which causes the leaflets to thicken and impairs the ability of the valve to fully open to permit forward blood flow. Valve regurgitation occurs when the leaflets of the valve do not close completely thereby causing blood to leak back into the prior chamber (e.g., causing blood to leak from the left ventricle to the left atrium).

There are three main mechanisms by which a native valve becomes regurgitant—or incompetent—which include Carpentier's type I, type II, and type III malfunctions. A Carpentier type I malfunction involves the dilation of the annulus such that normally functioning leaflets are distracted from each other and fail to form a tight seal (i.e., the leaflets do not coapt properly). Included in a type I mechanism malfunction are perforations of the leaflets, as are present in endocarditis. A Carpentier's type II malfunction involves prolapse of one or more leaflets of a native valve above a plane of coaptation. A Carpentier's type III malfunction involves restriction of the motion of one or more leaflets of a native valve such that the leaflets are abnormally constrained below the plane of the annulus. Leaflet restriction can be caused by rheumatic disease (Ma) or dilation of a ventricle (IIIb).

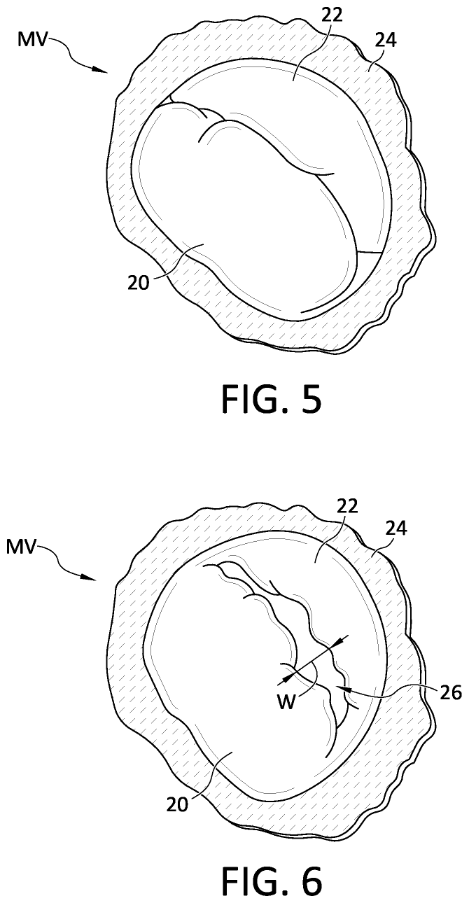

Referring to FIG. 5, when a healthy mitral valve MV is in a closed position, the anterior leaflet 20 and the posterior leaflet 22 coapt, which prevents blood from leaking from the left ventricle LV to the left atrium LA. Referring to FIGS. 3 and 6, mitral regurgitation MR occurs when the anterior leaflet 20 and/or the posterior leaflet 22 of the mitral valve MV is displaced into the left atrium LA during systole so that the edges of the leaflets 20, 22 are not in contact with each other. This failure to coapt causes a gap 26 between the anterior leaflet 20 and the posterior leaflet 22, which allows blood to flow back into the left atrium LA from the left ventricle LV during systole, as illustrated by the mitral regurgitation MR flow path shown in FIG. 3. Referring to FIG. 6, the gap 26 can have a width W between about 2.5 mm and about 17.5 mm, between about 5 mm and about 15 mm, between about 7.5 mm and about 12.5 mm, or about 10 mm. In some situations, the gap 26 can have a width W greater than 15 mm. As set forth above, there are several different ways that a leaflet (e.g., leaflets 20, 22 of mitral valve MV) may malfunction which can thereby lead to valvular regurgitation.

In any of the above-mentioned situations, a valve repair device or implant is desired that is capable of engaging the anterior leaflet 20 and the posterior leaflet 22 to close the gap 26 and prevent or inhibit regurgitation of blood through the mitral valve MV. As can be seen in FIG. 4, an abstract representation of a valve repair device, implantable device, or implant 10 is shown implanted between the leaflets 20, 22 such that regurgitation does not occur during systole (compare FIG. 3 with FIG. 4). In some implementations, the coaptation element (e.g., spacer, coaption element, gap filler, membrane, sheet, plug, wedge, balloon, etc.) of the device 10 has a generally tapered or triangular shape that naturally adapts to the native valve geometry and to its expanding leaflet nature (toward the annulus). In this application, the terms spacer, coaption element, coaptation element, and gap filler are used interchangeably and refer to an element that fills a portion of the space between native valve leaflets and/or that is configured such that the native valve leaflets engage or “coapt” against (e.g., such that the native leaflets coapt against the coaption element, coaptation element, spacer, etc. instead of only against one another).).

Although stenosis or regurgitation can affect any valve, stenosis is predominantly found to affect either the aortic valve AV or the pulmonary valve PV, and regurgitation is predominantly found to affect either the mitral valve MV or the tricuspid valve TV. Both valve stenosis and valve regurgitation increase the workload of the heart H and may lead to very serious conditions if left un-treated; such as endocarditis, congestive heart failure, permanent heart damage, cardiac arrest, and ultimately death. Because the left side of the heart (i.e., the left atrium LA, the left ventricle LV, the mitral valve MV, and the aortic valve AV) are primarily responsible for circulating the flow of blood throughout the body. Accordingly, because of the substantially higher pressures on the left side heart dysfunction of the mitral valve MV or the aortic valve AV is particularly problematic and often life threatening.

Malfunctioning native heart valves can either be repaired or replaced. Repair typically involves the preservation and correction of the patient's native valve. Replacement typically involves replacing the patient's native valve with a biological or mechanical substitute. Typically, the aortic valve AV and pulmonary valve PV are more prone to stenosis. Because stenotic damage sustained by the leaflets is irreversible, treatments for a stenotic aortic valve or stenotic pulmonary valve can be removal and replacement of the valve with a surgically implanted heart valve, or displacement of the valve with a transcatheter heart valve. The mitral valve MV and the tricuspid valve TV are more prone to deformation of leaflets and/or surrounding tissue, which, as described above, prevents the mitral valve MV or tricuspid valve TV from closing properly and allows for regurgitation or back flow of blood from the ventricle into the atrium (e.g., a deformed mitral valve MV may allow for regurgitation or back flow from the left ventricle LV to the left atrium LA as shown in FIG. 3). The regurgitation or back flow of blood from the ventricle to the atrium results in valvular insufficiency. Deformations in the structure or shape of the mitral valve MV or the tricuspid valve TV are often repairable. In addition, regurgitation can occur due to the chordae tendineae CT becoming dysfunctional (e.g., the chordae tendineae CT may stretch or rupture), which allows the anterior leaflet 20 and the posterior leaflet 22 to be reverted such that blood is regurgitated into the left atrium LA. The problems occurring due to dysfunctional chordae tendineae CT can be repaired by repairing the chordae tendineae CT or the structure of the mitral valve MV (e.g., by securing the leaflets 20, 22 at the affected portion of the mitral valve).

The devices and procedures disclosed herein often make reference to repairing the structure of a mitral valve. However, it should be understood that the devices and concepts provided herein can be used to repair any native valve, as well as any component of a native valve. Such devices can be used between the leaflets 20, 22 of the mitral valve MV to prevent or inhibit regurgitation of blood from the left ventricle into the left atrium. With respect to the tricuspid valve TV (FIG. 7), any of the devices and concepts herein can be used between any two of the anterior leaflet 30, septal leaflet 32, and posterior leaflet 34 to prevent or inhibit regurgitation of blood from the right ventricle into the right atrium. In addition, any of the devices and concepts provided herein can be used on all three of the leaflets 30, 32, 34 together to prevent or inhibit regurgitation of blood from the right ventricle to the right atrium. That is, the valve repair devices or implants provided herein can be centrally located between the three leaflets 30, 32, 34.

An example implantable device (e.g., implantable prosthetic device, etc.) or implant can optionally have a coaptation element (e.g., spacer, coaption element, gap filler, etc.) and at least one anchor (e.g., one, two, three, or more). In some implementations, an implantable device or implant can have any combination or sub-combination of the features disclosed herein without a coaptation element. When included, the coaptation element (e.g., coaption element, spacer, etc.) is configured to be positioned within the native heart valve orifice to help fill the space between the leaflets and form a more effective seal, thereby reducing or preventing or inhibiting regurgitation described above. The coaptation element can have a structure that is impervious to blood (or that resists blood flow therethrough) and that allows the native leaflets to close around the coaptation element during ventricular systole to block blood from flowing from the left or right ventricle back into the left or right atrium, respectively. The device or implant can be configured to seal against two or three native valve leaflets; that is, the device can be used in the native mitral (bicuspid) and tricuspid valves. The coaptation element is sometimes referred to herein as a spacer because the coaptation element can fill a space between improperly functioning native leaflets (e.g., mitral valve leaflets 20, 22 or tricuspid valve leaflets 30, 32, 34) that do not close completely.

The optional coaptation element (e.g., spacer, coaption element, etc.) can have various shapes. In some implementations, the coaptation element can have an elongated cylindrical shape having a round cross-sectional shape. In some implementations, the coaptation element can have an oval cross-sectional shape, an ovoid cross-sectional shape, a crescent cross-sectional shape, a rectangular cross-sectional shape, or various other non-cylindrical shapes. In some implementations, the coaptation element can have an atrial portion positioned in or adjacent to the atrium, a ventricular or lower portion positioned in or adjacent to the ventricle, and a side surface that extends between the native leaflets. In some implementations configured for use in the tricuspid valve, the atrial or upper portion is positioned in or adjacent to the right atrium, and the ventricular or lower portion is positioned in or adjacent to the right ventricle, and the side surface that extends between the native tricuspid leaflets.

In some implementations, the anchor can be configured to secure the device to one or both of the native leaflets such that the coaptation element is positioned between the two native leaflets. In some implementations configured for use in the tricuspid valve, the anchor is configured to secure the device to one, two, or three of the tricuspid leaflets such that the coaptation element is positioned between the three native leaflets. In some implementations, the anchor can attach to the coaptation element at a location adjacent the ventricular portion of the coaptation element. In some implementations, the anchor can attach to an actuation element, such as a shaft or actuation wire, to which the coaptation element is also attached. In some implementations, the anchor and the coaptation element can be positioned independently with respect to each other by separately moving each of the anchor and the coaptation element along the longitudinal axis of the actuation element (e.g., actuation shaft, actuation rod, actuation tube, actuation wire, etc.). In some implementations, the anchor and the coaptation element can be positioned simultaneously by moving the anchor and the coaptation element together along the longitudinal axis of the actuation element, e.g., shaft, actuation wire, etc. The anchor can be configured to be positioned behind a native leaflet when implanted such that the leaflet is grasped by the anchor.

The device or implant can be configured to be implanted via a delivery system or other means for delivery. The delivery system can comprise one or more of a guide/delivery sheath, a delivery catheter, a steerable catheter, an implant catheter, tube, combinations of these, etc. The coaptation element and the anchor can be compressible to a radially compressed state and can be self-expandable to a radially expanded state when compressive pressure is released. The device can be configured for the anchor to be expanded radially away from the still-compressed coaptation element initially in order to create a gap between the coaptation element and the anchor. A native leaflet can then be positioned in the gap. The coaptation element can be expanded radially, closing the gap between the coaptation element and the anchor and capturing the leaflet between the coaptation element and the anchor. In some implementations, the anchor and coaptation element are optionally configured to self-expand. The implantation methods for various implementations can be different and are more fully discussed below with respect to each implementation. Additional information regarding these and other delivery methods can be found in U.S. Pat. No. 8,449,599 and U.S. Patent Application Publication Nos. 2014/0222136, 2014/0067052, 2016/0331523, and PCT patent application publication Nos. WO2020/076898, each of which is incorporated herein by reference in its entirety for all purposes. These method(s) can be performed on a living animal or on a simulation, such as on a cadaver, cadaver heart, simulator (e.g., with the body parts, heart, tissue, etc. being simulated), etc. mutatis mutandis.

The disclosed devices or implants can be configured such that the anchor is connected to a leaflet, taking advantage of the tension from native chordae tendineae to resist high systolic pressure urging the device toward the left atrium. During diastole, the devices can rely on the compressive and retention forces exerted on the leaflet that is grasped by the anchor.

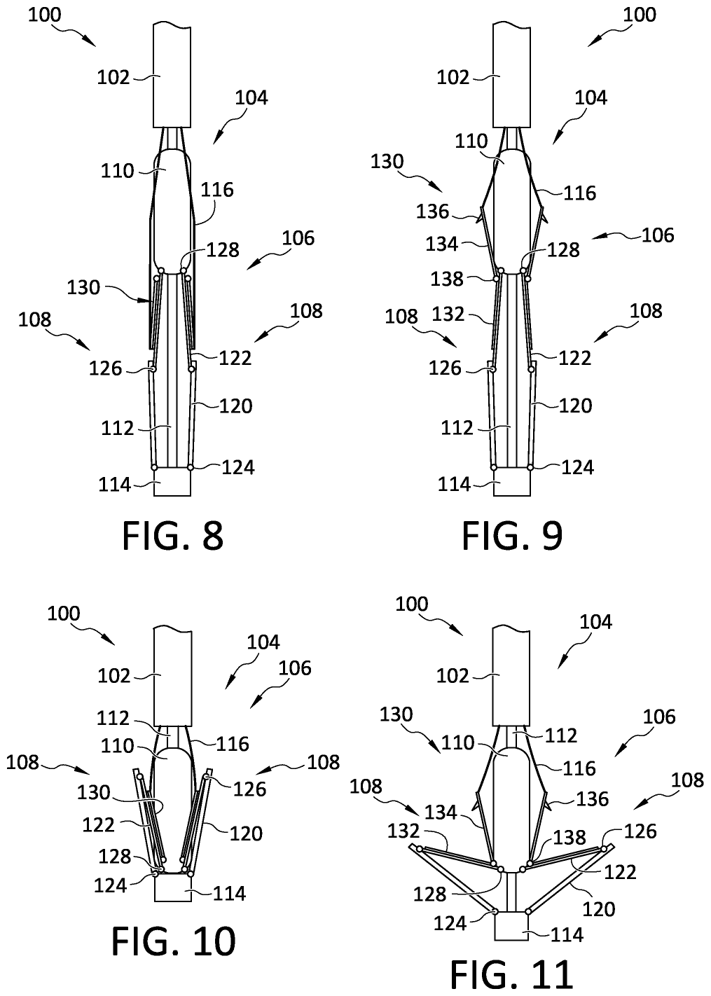

Referring now to FIGS. 8-15, a schematically illustrated device or implant 100 (e.g., a prosthetic spacer device, valve repair device implantable device, etc.) is shown in various stages of deployment. The device or implant 100 and other similar devices/implants are described in more detail in PCT patent application publication Nos. WO2018/195215, WO2020/076898, and WO 2019/139904, which are incorporated herein by reference in their entirety. The device 100 can include any other features for another device or implant discussed in the present application or the applications cited above, and the device 100 can be positioned to engage valve tissue (e.g., leaflets 20, 22, 30, 32, 34) as part of any suitable valve repair system (e.g., any valve repair system disclosed in the present application or the applications cited above).

The device or implant 100 is deployed from a delivery system or other means for delivery 102. The delivery system 102 can comprise one or more of a catheter, a sheath, a guide catheter/sheath, a delivery catheter/sheath, a steerable catheter, an implant catheter, a tube, a channel, a pathway, combinations of these, etc. The device or implant 100 includes a coaptation portion 104 and an anchor portion 106.

In some implementations, the coaptation portion 104 of the device or implant 100 includes a coaptation element 110 (e.g., spacer, plug, filler, foam, sheet, membrane, coaption element, etc.) that is adapted to be implanted between leaflets of a native valve (e.g., a native mitral valve, native tricuspid valve, etc.) and is slidably attached to an actuation element 112 (e.g., actuation wire, shaft, tube, hypotube, line, suture, braid, etc.). The anchor portion 106 includes one or more anchors 108 that are actuatable between open and closed conditions and can take a wide variety of forms, such as, for example, paddles, gripping elements, or the like. Actuation of the means for actuating or actuation element 112 opens and closes the anchor portion 106 of the device 100 to grasp the native valve leaflets during implantation. The means for actuating or actuation element 112 (as well as other means for actuating and actuation elements herein) can take a wide variety of different forms (e.g., as a wire, rod, shaft, tube, screw, suture, line, strip, combination of these, etc.), be made of a variety of different materials, and have a variety of configurations. As one example, the actuation element can be threaded such that rotation of the actuation element moves the anchor portion 106 relative to the coaptation portion 104. Or, the actuation element can be unthreaded, such that pushing or pulling the actuation element 112 moves the anchor portion 106 relative to the coaptation portion 104.

The anchor portion 106 and/or anchors of the device 100 include outer paddles 120 and inner paddles 122 that are, in some implementations, connected between a cap 114 and the coaptation element 110 by portions 124, 126, 128. The portions 124, 126, 128 can be jointed and/or flexible to move between all of the positions described below. The interconnection of the outer paddles 120, the inner paddles 122, the coaptation element 110, and the cap 114 by the portions 124, 126, and 128 can constrain the device to the positions and movements illustrated herein.

In some implementations, the delivery system 102 includes a steerable catheter, implant catheter, and means for actuating or actuation element 112 (e.g., actuation wire, actuation shaft, etc.). These can be configured to extend through a guide catheter/sheath (e.g., a transseptal sheath, etc.). In some implementations, the means for actuating or actuation element 112 extends through a delivery catheter and the coaptation element 110 to the distal end (e.g., a cap 114 or other attachment portion at the distal connection of the anchor portion 106). Extending and retracting the actuation element 112 increases and decreases the spacing between the coaptation element 110 and the distal end of the device (e.g., the cap 114 or other attachment portion), respectively. In some implementations, a collar or other attachment element (e.g., clamp, clip, lock, sutures, friction fit, buckle, snap fit, lasso, etc.) removably attaches the coaptation element 110 to the delivery system 102, either directly or indirectly, so that the means for actuating or actuation element 112 slides through the collar or other attachment element and, in some implementations, through a coaptation element 110 during actuation to open and close the paddles 120, 122 of the anchor portion 106 and/or anchors 108.

In some implementation, the anchor portion 106 and/or anchors 108 can include attachment portions or gripping members. The illustrated gripping members can comprise clasps 130 that include a base or fixed arm 132, a movable arm 134, optional barbs, friction-enhancing elements, or other means for securing 136 (e.g., protrusions, ridges, grooves, textured surfaces, adhesive, etc.), and a joint portion 138. The fixed arms 132 are attached to the inner paddles 122. In some implementations, the fixed arms 132 are attached to the inner paddles 122 with the joint portion 138 disposed proximate a coaptation element 110. In some implementations, the clasps (e.g., barbed clasps, barbed gripping members, etc.) have flat surfaces and do not fit in a recess of the inner paddle. Rather, the flat portions of the clasps are disposed against the surface of the inner paddle 122. The joint portion 138 provides a spring force between the fixed and movable arms 132, 134 of the clasp 130. The joint portion 138 can be any suitable joint, such as a flexible joint, a spring joint, a pivot joint, or the like. In some implementations, the joint portion 138 is a flexible piece of material integrally formed with the fixed and movable arms 132, 134. The fixed arms 132 are attached to the inner paddles 122 and remain stationary or substantially stationary relative to the inner paddles 122 when the movable arms 134 are opened to open the clasps 130 and expose the optional barbs, friction-enhancing elements, or means for securing 136.

In some implementations, the clasps 130 are opened by applying tension to actuation lines 116 attached to the movable arms 134, thereby causing the movable arms 134 to articulate, flex, or pivot on the joint portions 138. The actuation lines 116 extend through the delivery system 102 (e.g., through a steerable catheter and/or an implant catheter). Other actuation mechanisms are also possible.

The actuation line 116 can take a wide variety of forms, such as, for example, a line, a suture, a wire, a rod, a catheter, or the like. The clasps 130 can be spring loaded so that in the closed position the clasps 130 continue to provide a pinching force on the grasped native leaflet. This pinching force remains constant regardless of the position of the inner paddles 122. Optional barbs, friction-enhancing elements, or other means for securing 136 of the clasps 130 can grab, pinch, and/or pierce the native leaflets to further secure the native leaflets.

During implantation, the paddles 120, 122 can be opened and closed, for example, to grasp the native leaflets (e.g., native mitral valve leaflets, etc.) between the paddles 120, 122 and/or between the paddles 120, 122 and a coaptation element 110. The clasps 130 can be used to grasp and/or further secure the native leaflets by engaging the leaflets with optional barbs, friction-enhancing elements, or means for securing 136 and pinching the leaflets between the movable and fixed arms 134, 132. The optional barbs, friction-enhancing elements, or other means for securing 136 (e.g., protrusions, ridges, grooves, textured surfaces, adhesive, etc.) of the clasps 130 increase friction with the leaflets or can partially or completely puncture the leaflets. The actuation lines 116 can be actuated separately so that each clasp 130 can be opened and closed separately. Separate operation allows one leaflet to be grasped at a time, or for the repositioning of a clasp 130 on a leaflet that was insufficiently grasped, without altering a successful grasp on the other leaflet. The clasps 130 can be opened and closed relative to the position of the inner paddle 122 (as long as the inner paddle is in an open or at least partially open position), thereby allowing leaflets to be grasped in a variety of positions as the particular situation requires.

Referring now to FIG. 8, the device 100 is shown in an elongated or fully open condition for deployment from an implant delivery catheter of the delivery system 102. The device 100 is disposed at the end of the catheter in the fully open position, because the fully open position takes up the least space and allows the smallest catheter to be used (or the largest device to be used for a given catheter size). In the elongated condition the cap 114 is spaced apart from the coaptation element 110 such that the paddles 120, 122 are fully extended. In some implementations, an angle formed between the interior of the outer and inner paddles 120, 122 is approximately 180 degrees. The clasps 130 are kept in a closed condition during deployment through the delivery system 102 so that the optional barbs, friction-enhancing elements, or other means for securing 136 (FIG. 9) do not catch or damage the delivery system 102 or tissue in the patient's heart.

Referring now to FIG. 9, the device 100 is shown in an elongated detangling condition, similar to FIG. 8, but with the clasps 130 in a fully open position, ranging from about 140 degrees to about 200 degrees, from about 170 degrees to about 190 degrees, or about 180 degrees between fixed and movable arms 132, 134 of the clasps 130. Fully opening the paddles 120, 122 and the clasps 130 has been found to improve ease of detanglement or detachment from anatomy of the patient, such as the chordae tendineae CT, during implantation of the device 100.

Referring now to FIG. 10, the device 100 is shown in a shortened or fully closed condition. The compact size of the device 100 in the shortened condition allows for easier maneuvering and placement within the heart. To move the device 100 from the elongated condition to the shortened condition, the means for actuating or actuation element 112 is retracted to pull the cap 114 towards the coaptation element 110. The connection portion(s) 126 (e.g., joint(s), flexible connection(s), etc.) between the outer paddle 120 and inner paddle 122 are constrained in movement such that compression forces acting on the outer paddle 120 from the cap 114 being retracted towards the coaptation element 110 cause the paddles or gripping elements to move radially outward. During movement from the open to closed position, the outer paddles 120 maintain an acute angle with the means for actuating or actuation element 112. The outer paddles 120 can optionally be biased toward a closed position. The inner paddles 122 during the same motion move through a considerably larger angle as they are oriented away from the coaptation element 110 in the open condition and collapse along the sides of the coaptation element 110 in the closed condition. In some implementations, the inner paddles 122 are thinner and/or narrower than the outer paddles 120, and the connection portions 126, 128 (e.g., joints, flexible connections, etc.) connected to the inner paddles 122 can be thinner and/or more flexible. For example, this increased flexibility can allow more movement than the connection portion 124 connecting the outer paddle 120 to the cap 114. In some implementations, the outer paddles 120 are narrower than the inner paddles 122. The connection portions 126, 128 connected to the inner paddles 122 can be more flexible, for example, to allow more movement than the connection portion 124 connecting the outer paddle 120 to the cap 114. In some implementations, the inner paddles 122 can be the same or substantially the same width as the outer paddles

Referring now to FIGS. 11-13, the device 100 is shown in a partially open, grasp-ready condition. To transition from the fully closed to the partially open condition, the means for actuating or actuation element (e.g., actuation wire, actuation shaft, etc.) is extended to push the cap 114 away from the coaptation element 110, thereby pulling on the outer paddles 120, which in turn pull on the inner paddles 122, causing the anchors or anchor portion 106 to partially unfold. The actuation lines 116 are also retracted to open the clasps 130 so that the leaflets can be grasped. In some implementations, the pair of inner and outer paddles 122, 120 are moved in unison, rather than independently, by a single means for actuating or single actuation element 112. Also, the positions of the clasps 130 are dependent on the positions of the paddles 122, 120. For example, referring to FIG. 10 closing the paddles 122, 120 also closes the clasps. In some implementations, the paddles 120, 122 can be independently controllable. For example, the device 100 can have two actuation elements and two independent caps (or other attachment portions), such that one independent actuation element (e.g., wire, shaft, etc.) and cap (or other attachment portion) are used to control one paddle, and the other independent actuation element and cap (or other attachment portion) are used to control the other paddle.

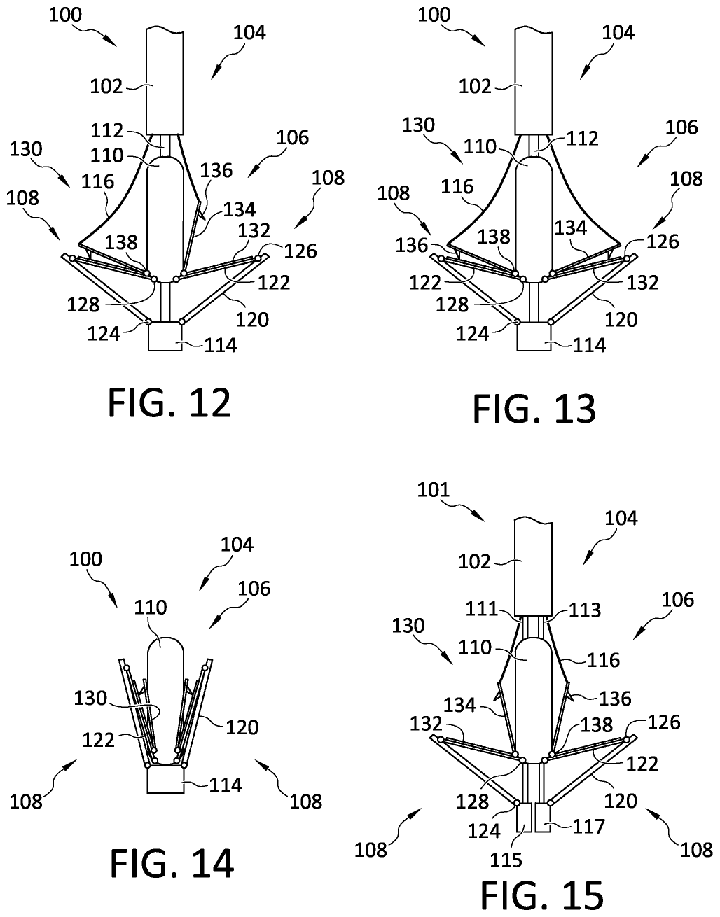

Referring now to FIG. 12, one of the actuation lines 116 is extended to allow one of the clasps 130 to close. Referring now to FIG. 13, the other actuation line 116 is extended to allow the other clasp 130 to close. Either or both of the actuation lines 116 can be repeatedly actuated to repeatedly open and close the clasps 130.

Referring now to FIG. 14, the device 100 is shown in a fully closed and deployed condition. The delivery system or means for delivery 102 and means for actuating or actuation element 112 are retracted and the paddles 120, 122 and clasps 130 remain in a fully closed position. Once deployed, the device 100 can be maintained in the fully closed position with a mechanical latch or can be biased to remain closed through the use of spring materials, such as steel, other metals, plastics, composites, etc. or shape-memory alloys such as Nitinol. For example, the connection portions 124, 126, 128, the joint portions 138, and/or the inner and outer paddles 122, and/or an additional biasing component (not shown) can be formed of metals such as steel or shape-memory alloy, such as Nitinol—produced in a wire, sheet, tubing, or laser sintered powder—and are biased to hold the outer paddles 120 closed around the coaptation element 110 and the clasps 130 pinched around native leaflets. Similarly, the fixed and movable arms 132, 134 of the clasps 130 are biased to pinch the leaflets. In some implementations, the attachment or connection portions 124, 126, 128, joint portions 138, and/or the inner and outer paddles 122, and/or an additional biasing component (not shown) can be formed of any other suitably elastic material, such as a metal or polymer material, to maintain the device 100 in the closed condition after implantation.

FIG. 15 illustrates an example where the paddles 120, 122 are independently controllable. The device 101 illustrated by FIG. 15 is similar to the device 100 illustrated by FIG. 11, except the device 101 of FIG. 15 includes an actuation element that is configured as two independent actuation elements 111, 113 that are coupled to two independent caps 115, 117. To transition a first inner paddle 122 and a first outer paddle 120 from the fully closed to the partially open condition, the means for actuating or actuation element 111 is extended to push the cap 115 away from the coaptation element 110, thereby pulling on the outer paddle 120, which in turn pulls on the inner paddle 122, causing the first anchor 108 to partially unfold. To transition a second inner paddle 122 and a second outer paddle 120 from the fully closed to the partially open condition, the means for actuating or actuation element 113 is extended to push the cap 115 away from the coaptation element 110, thereby pulling on the outer paddle 120, which in turn pulls on the inner paddle 122, causing the second anchor 108 to partially unfold. The independent paddle control illustrated by FIG. 15 can be implemented on any of the devices disclosed by the present application. For comparison, in the example illustrated by FIG. 11, the pair of inner and outer paddles 122, 120 are moved in unison, rather than independently, by a single means for actuating or actuation element 112.

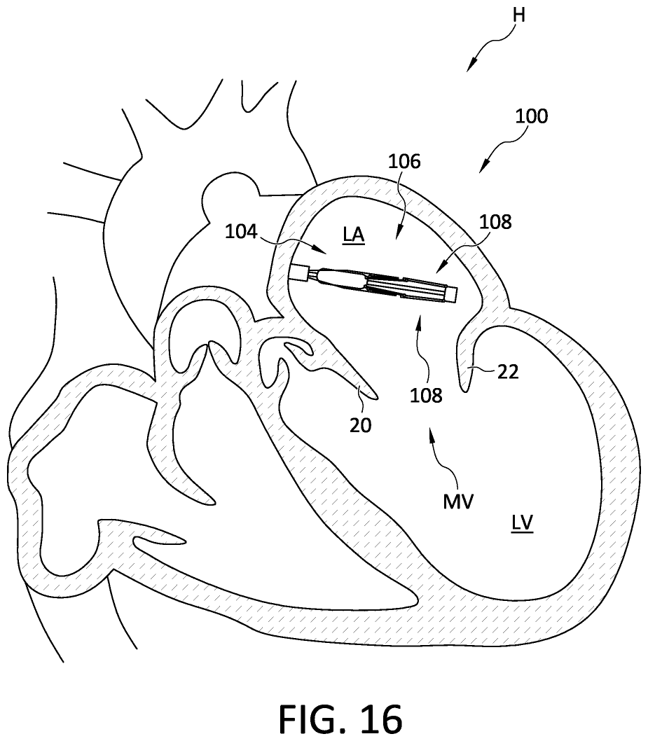

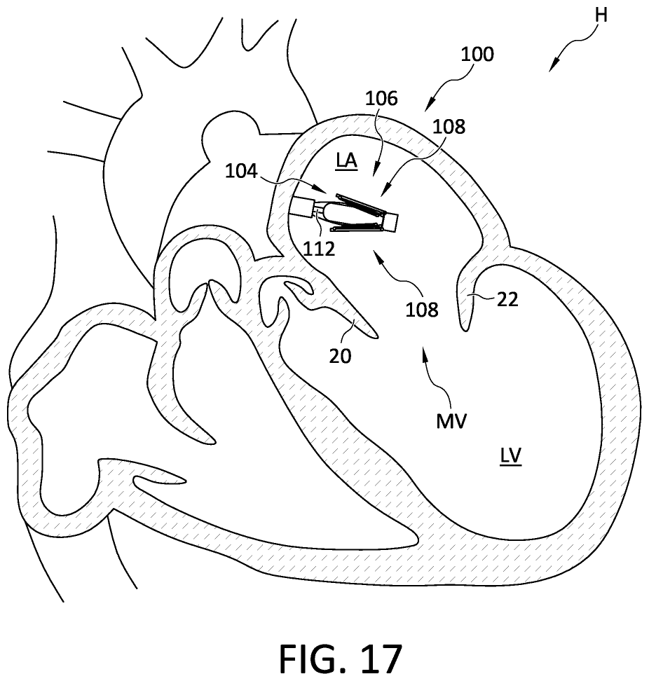

Referring now to FIGS. 16-21, the device 100 of FIGS. 8-14 is shown being delivered and deployed within the native mitral valve MV of the heart H. Referring to FIG. 16, a delivery sheath/catheter is inserted into the left atrium LA through the septum and the implant/device 100 is deployed from the delivery catheter/sheath in the fully open condition as illustrated in FIG. 16. The means for actuating or actuation element 112 is then retracted to move the implant/device into the fully closed condition shown in FIG. 17.

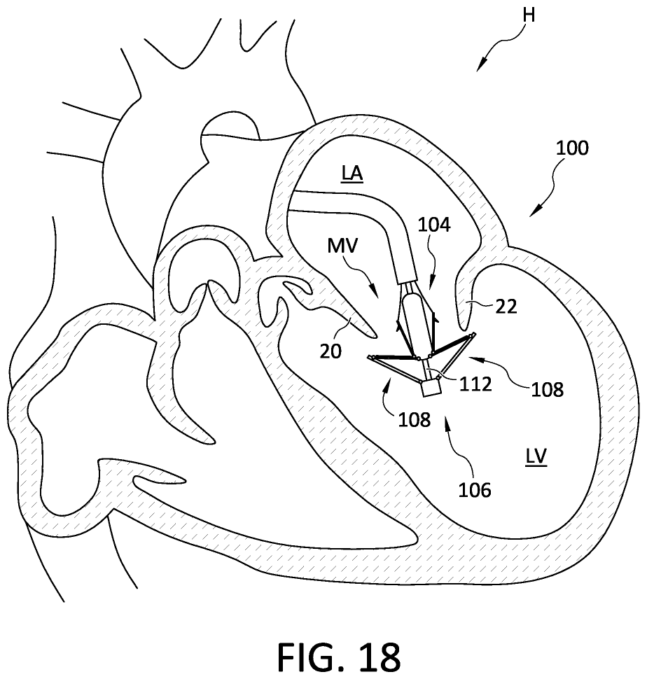

As can be seen in FIG. 18, the implant/device is moved into position within the mitral valve MV into the ventricle LV and partially opened so that the leaflets 20, 22 can be grasped. For example, a steerable catheter can be advanced and steered or flexed to position the steerable catheter as illustrated by FIG. 18. The implant catheter connected to the implant/device can be advanced from inside the steerable catheter to position the implant as illustrated by FIG. 18.

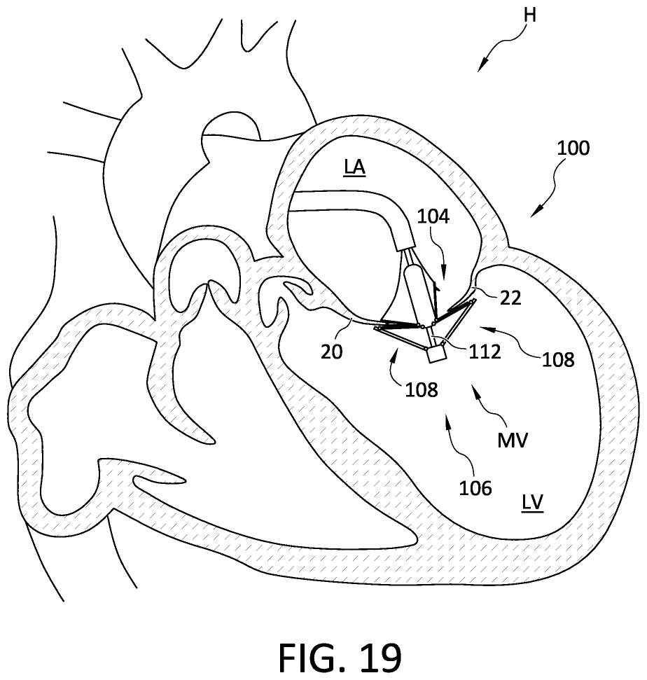

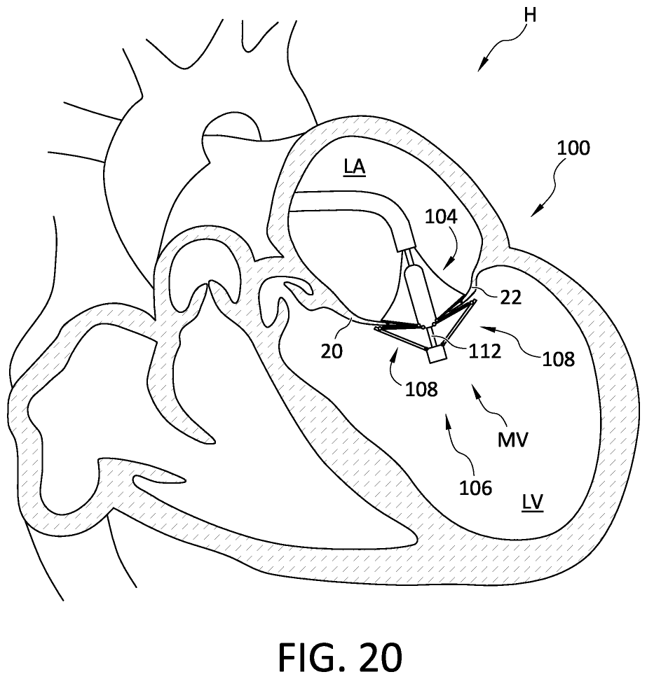

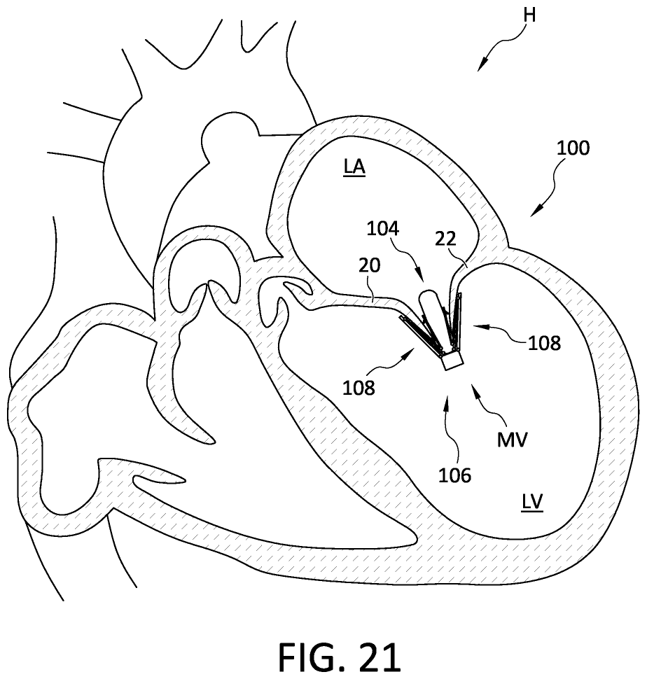

Referring now to FIG. 19, the implant catheter can be retracted into the steerable catheter to position the mitral valve leaflets 20, 22 in the clasps 130. An actuation line 116 is extended to close one of the clasps 130, capturing a leaflet 20. FIG. 20 shows the other actuation line 116 being then extended to close the other clasp 130, capturing the remaining leaflet 22. Lastly, as can be seen in FIG. 21, the delivery system 102 (e.g., steerable catheter, implant catheter, etc.), means for actuating or actuation element 112 and actuation lines 116 are then retracted and the device or implant 100 is fully closed and deployed in the native mitral valve MV.

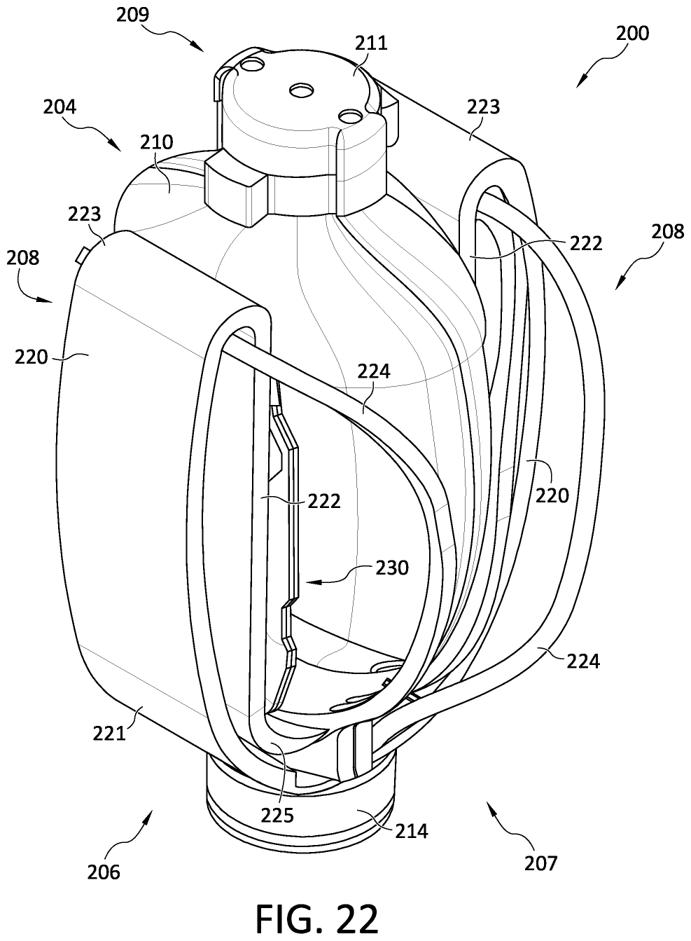

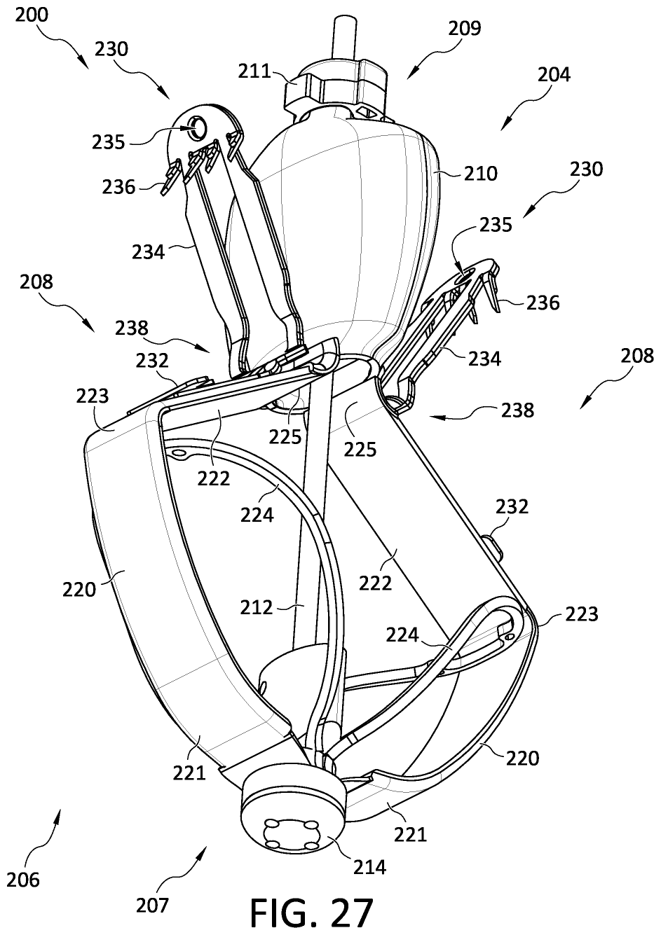

Referring now to FIGS. 22-27, an example of an implantable device or implant or implant 200 is shown. The implantable device 200 is one of the many different configurations that the device 100 that is schematically illustrated in FIGS. 8-14 can take. The device 200 can include any other features for an implantable device or implant discussed in the present application, and the device 200 can be positioned to engage valve tissue 20, 22 as part of any suitable valve repair system (e.g., any valve repair system disclosed in the present application). The device/implant 200 can be a prosthetic spacer device, valve repair device, or another type of implant that attaches to leaflets of a native valve.

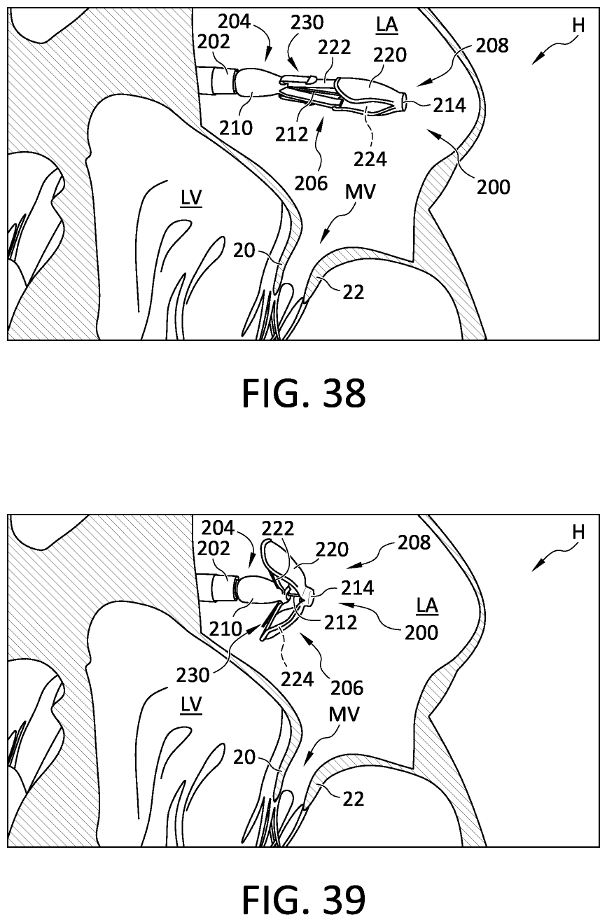

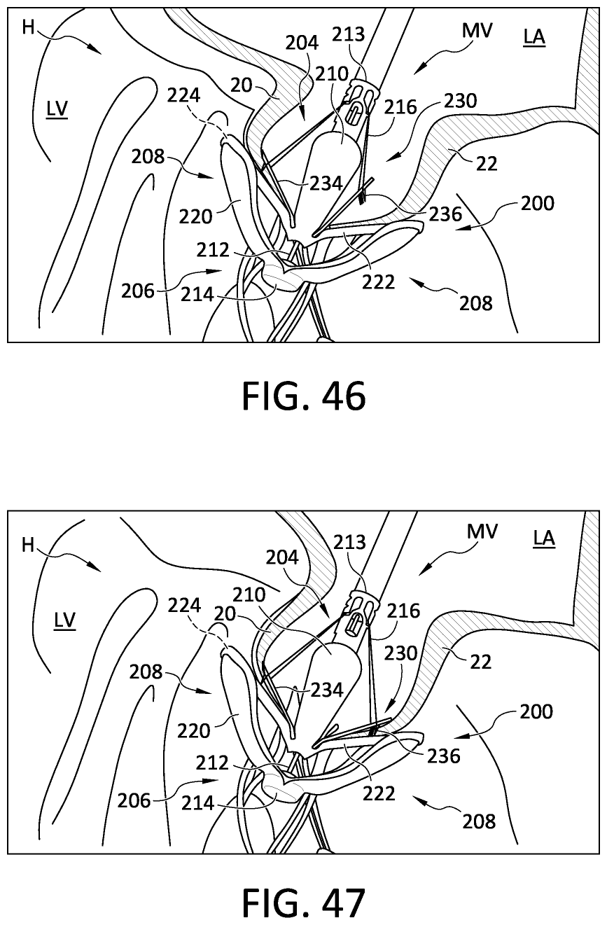

In some implementations, the implantable device or implant 200 includes a coaptation portion 204, a proximal or attachment portion 205, an anchor portion 206, and a distal portion 207. In some implementations, the coaptation portion 204 of the device optionally includes a coaptation element 210 (e.g., a spacer, coaption element, plug, membrane, sheet, etc.) for implantation between leaflets of a native valve. In some implementations, the anchor portion 206 includes a plurality of anchors 208. The anchors can be configured in a variety of ways. In some implementations, each anchor 208 includes outer paddles 220, inner paddles 222, paddle extension members or paddle frames 224, and clasps 230. In some implementations, the attachment portion 205 includes a first or proximal collar 211 (or other attachment element) for engaging with a capture mechanism 213 (see e.g., FIGS. 43-49) of a delivery system 202 (FIGS. 38-42 and 49). Delivery system 202 can be the same as or similar to delivery system 102 described elsewhere and can comprise one or more of a catheter, a sheath, a guide catheter/sheath, a delivery catheter/sheath, a steerable catheter, an implant catheter, a tube, a channel, a pathway, combinations of these, etc. The capture mechanism can be configured in a variety of ways and, in some implementations, can comprise one or more of a clamp, clip, pin, suture, line, lasso, noose, snare, buckle, lock, latch, etc.

In some implementations, the coaptation element 210 and paddles 220,222 are formed from a flexible material that can be a metal fabric, such as a mesh, woven, braided, or formed in any other suitable way or a laser cut or otherwise cut flexible material. The material can be cloth, shape-memory alloy wire—such as Nitinol—to provide shape-setting capability, or any other flexible material suitable for implantation in the human body.

An actuation element 212 (e.g., actuation shaft, actuation rod, actuation tube, actuation wire, actuation line, etc.) extends from the delivery system 202 to engage and enable actuation of the implantable device or implant 200. In some implementations, the actuation element 212 extends through the capture mechanism 213, proximal collar 211, and coaptation element 210 to engage a cap 214 of the distal portion 207. The actuation element 212 can be configured to removably engage the cap 214 with a threaded connection, or the like, so that the actuation element 212 can be disengaged and removed from the device 200 after implantation.

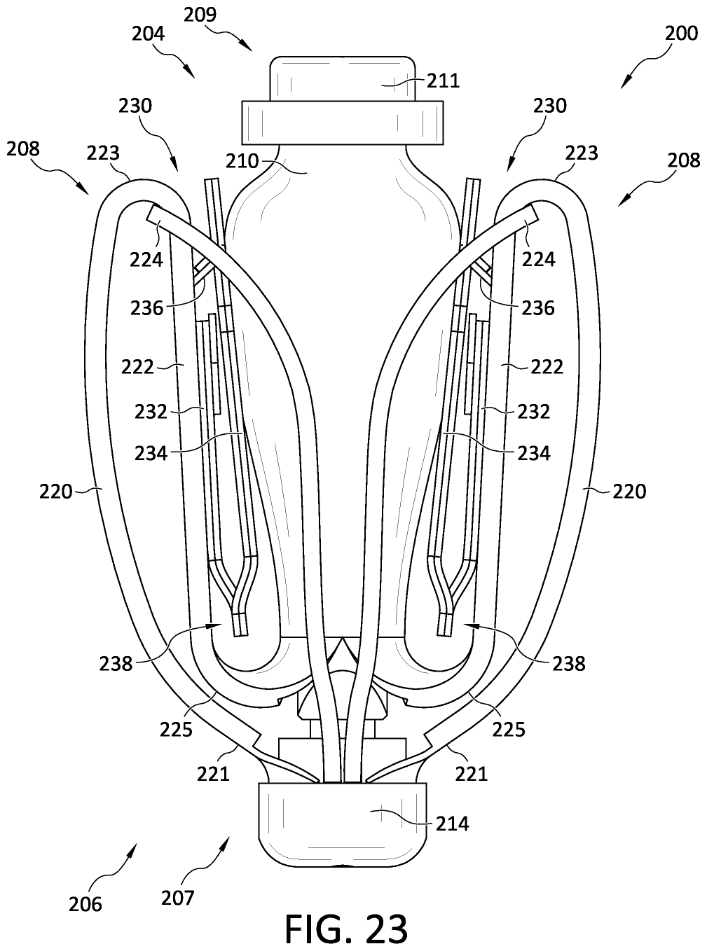

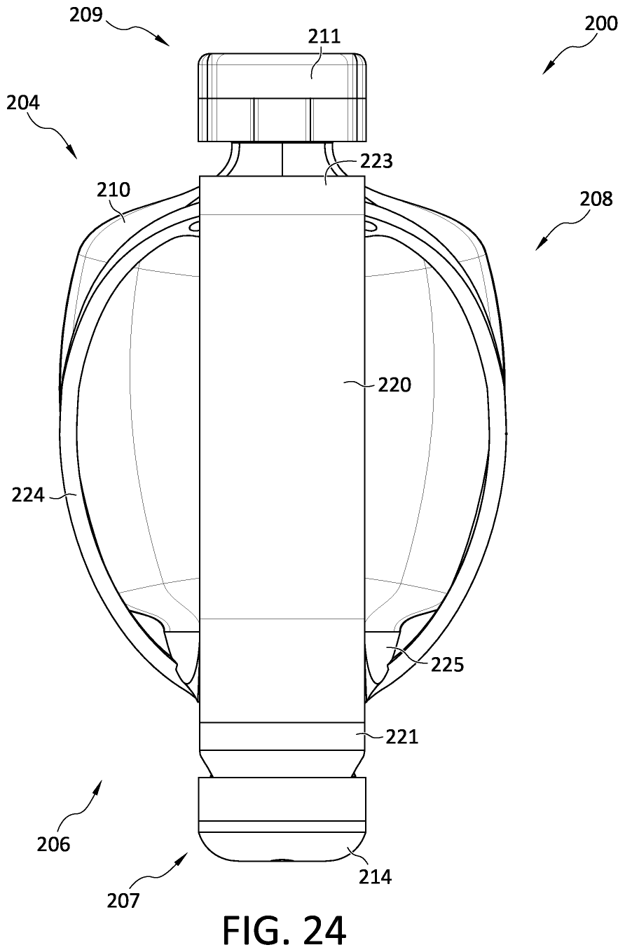

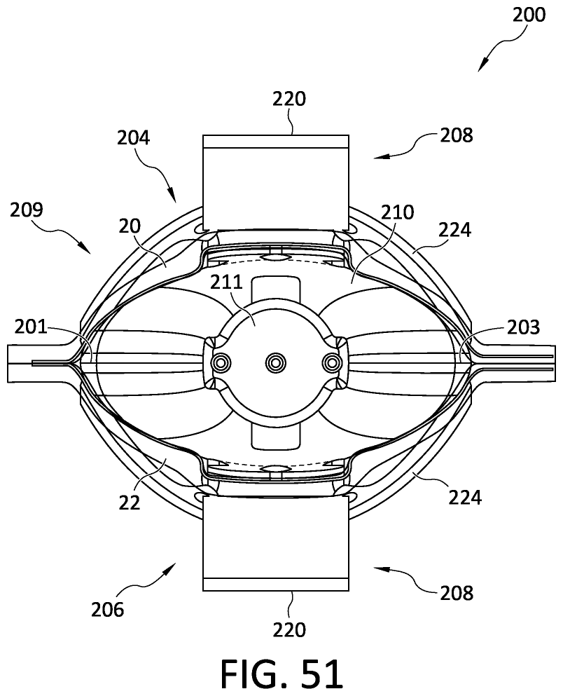

The coaptation element 210 extends from the proximal collar 211 (or other attachment element) to the inner paddles 222. In some implementations, the coaptation element 210 has a generally elongated and round shape, though other shapes and configurations are possible. In some implementations, the coaptation element 210 has an elliptical shape or cross-section when viewed from above (e.g., FIG. 51) and has a tapered shape or cross-section when seen from a front view (e.g., FIG. 23) and a round shape or cross-section when seen from a side view (e.g., FIG. 24). A blend of these three geometries can result in the three-dimensional shape of the illustrated coaptation element 210 that achieves the benefits described herein. The round shape of the coaptation element 210 can also be seen, when viewed from above, to substantially follow or be close to the shape of the paddle frames 224.

The size and/or shape of the coaptation element 210 can be selected to minimize the number of implants that a single patient will require (preferably one), while at the same time maintaining low transvalvular gradients. In some implementations, the anterior-posterior distance at the top of the coaptation element is about 5 mm, and the medial-lateral distance of the coaptation element at its widest is about 10 mm. In some implementations, the overall geometry of the device 200 can be based on these two dimensions and the overall shape strategy described above. It should be readily apparent that the use of other anterior-posterior distance anterior-posterior distance and medial-lateral distance as starting points for the device will result in a device having different dimensions. Further, using other dimensions and the shape strategy described above will also result in a device having different dimensions.

In some implementations, the outer paddles 220 are jointably attached to the cap 214 of the distal portion 207 by connection portions 221 and to the inner paddles 222 by connection portions 223. The inner paddles 222 are jointably attached to the coaptation element by connection portions 225. In this manner, the anchors 208 are configured similar to legs in that the inner paddles 222 are like upper portions of the legs, the outer paddles 220 are like lower portions of the legs, and the connection portions 223 are like knee portions of the legs.

In some implementations, the inner paddles 222 are stiff, relatively stiff, rigid, have rigid portions and/or are stiffened by a stiffening member or a fixed arm 232 of the clasps 230. The stiffening of the inner paddle allows the device to move to the various different positions shown and described herein. The inner paddle 222, the outer paddle 220, the coaptation can all be interconnected as described herein, such that the device 200 is constrained to the movements and positions shown and described herein.

In some implementations, the paddle frames 224 are attached to the cap 214 at the distal portion 207 and extend to the connection portions 223 between the inner and outer paddles 222, 220. In some implementations, the paddle frames 224 are formed of a material that is more rigid and stiff than the material forming the paddles 222, 220 so that the paddle frames 224 provide support for the paddles 222, 220.

The paddle frames 224 provide additional pinching force between the inner paddles 222 and the coaptation element 210 and assist in wrapping the leaflets around the sides of the coaptation element 210 for a better seal between the coaptation element 210 and the leaflets, as can be seen in FIG. 51. That is, the paddle frames 224 can be configured with a round three-dimensional shape extending from the cap 214 to the connection portions 223 of the anchors 208. The connections between the paddle frames 224, the outer and inner paddles 220, 222, the cap 214, and the coaptation element 210 can constrain each of these parts to the movements and positions described herein. In particular the connection portion 223 is constrained by its connection between the outer and inner paddles 220, 222 and by its connection to the paddle frame 224. Similarly, the paddle frame 224 is constrained by its attachment to the connection portion 223 (and thus the inner and outer paddles 222, 220) and to the cap 214.

Configuring the paddle frames 224 in this manner provides increased surface area compared to the outer paddles 220 alone. This can, for example, make it easier to grasp and secure the native leaflets. The increased surface area can also distribute the clamping force of the paddles 220 and paddle frames 224 against the native leaflets over a relatively larger surface of the native leaflets in order to further protect the native leaflet tissue. Referring again to FIG. 51, the increased surface area of the paddle frames 224 can also allow the native leaflets to be clamped to the implantable device or implant 200, such that the native leaflets coapt entirely around the coaptation member or coaptation element 210. This can, for example, improve sealing of the native leaflets 20, 22 and thus prevent or further reduce mitral regurgitation.

In some implementations the clasps comprise a movable arm coupled to the anchors. In some implementations, the clasps 230 include a base or fixed arm 232, a movable arm 234, optional barbs 236, and a joint portion 238. The fixed arms 232 are attached to the inner paddles 222, with the joint portion 238 disposed proximate the coaptation element 210. The joint portion 238 is spring-loaded so that the fixed and movable arms 232, 234 are biased toward each other when the clasp 230 is in a closed condition. In some implementations, the clasps 230 include friction-enhancing elements or means for securing, such as optional barbs, protrusions, ridges, grooves, textured surfaces, adhesive, etc.

In some implementations, the fixed arms 232 are attached to the inner paddles 222 through holes or slots 231 with sutures (not shown). The fixed arms 232 can be attached to the inner paddles 222 with any suitable means, such as screws or other fasteners, crimped sleeves, mechanical latches or snaps, welding, adhesive, clamps, latches, or the like. The fixed arms 232 remain substantially stationary relative to the inner paddles 222 when the movable arms 234 are opened to open the clasps 230 and expose the optional barbs or other friction-enhancing elements 236. The clasps 230 are opened by applying tension to actuation lines 216 (e.g., as shown in FIGS. 43-48) attached to holes 235 in the movable arms 234, thereby causing the movable arms 234 to articulate, pivot, and/or flex on the joint portions 238.

Referring now to FIG. 29, a close-up view of one of the leaflets 20, 22 grasped by a clasp such as clasp 230 is shown. The leaflet 20, 22 is grasped between the movable and fixed arms 234, 232 of the clasp 230. The tissue of the leaflet 20, 22 is not pierced by the optional barbs or friction-enhancing elements 236, though in some implementations the optional barbs 236 can partially or fully pierce through the leaflet 20, 22. The angle and height of the optional barbs or friction-enhancing elements 236 relative to the movable arm 234 helps to secure the leaflet 20, 22 within the clasp 230. In particular, a force pulling the implant off of the native leaflet 20, 22 will encourage the optional barbs or friction-enhancing elements 236 to further engage the tissue, thereby ensuring better retention. Retention of the leaflet 20, 22 in the clasp 230 is further improved by the position of fixed arm 232 near the optional barbs/friction-enhancing elements 236 when the clasp 230 is closed. In this arrangement, the tissue is formed by the fixed arms 232 and the movable arms 234 and the optional barbs/friction-enhancing elements 236 into an S-shaped torturous path. Thus, forces pulling the leaflet 20, 22 away from the clasp 230 will encourage the tissue to further engage the optional barbs/friction-enhancing elements 236 before the leaflets 20, 22 can escape. For example, leaflet tension during diastole can encourage the optional barbs 236 to pull toward the end portion of the leaflet 20, 22. Thus, the S-shaped path can utilize the leaflet tension during diastole to more tightly engage the leaflets 20, 22 with the optional barbs/friction-enhancing elements 236.

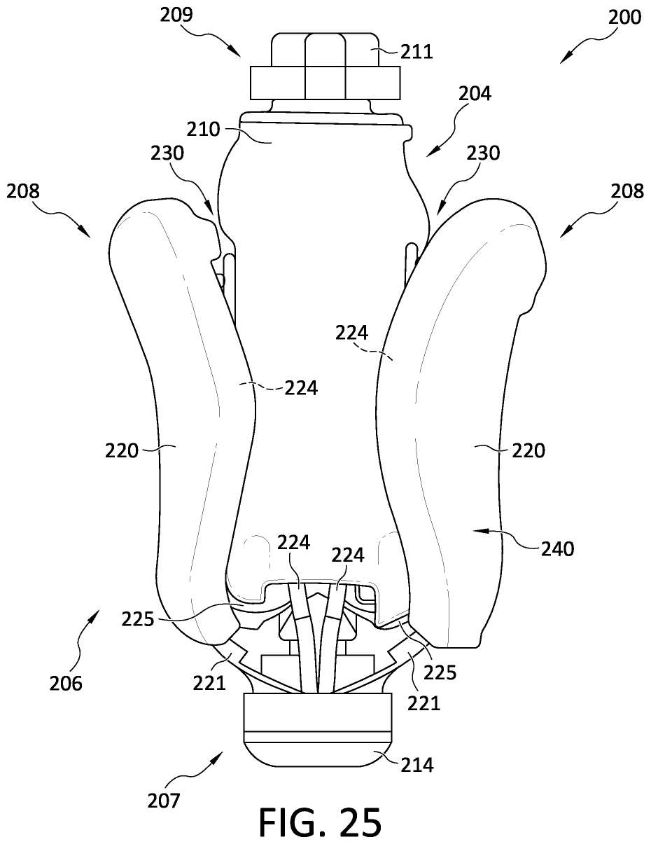

Referring to FIG. 25, the device or implant 200 can also include a cover 240. In some implementations, the cover 240 can be disposed on the coaptation element 210, the outer and inner paddles 220, 222, and/or the paddle frames 224. The cover 240 can be configured to prevent or reduce blood-flow through the device or implant 200 and/or to promote native tissue ingrowth. In some implementations, the cover 240 can be a cloth or fabric such as PET, velour, or other suitable fabric. In some implementations, in lieu of or in addition to a fabric, the cover 240 can include a coating (e.g., polymeric) that is applied to the implantable device or implant 200.

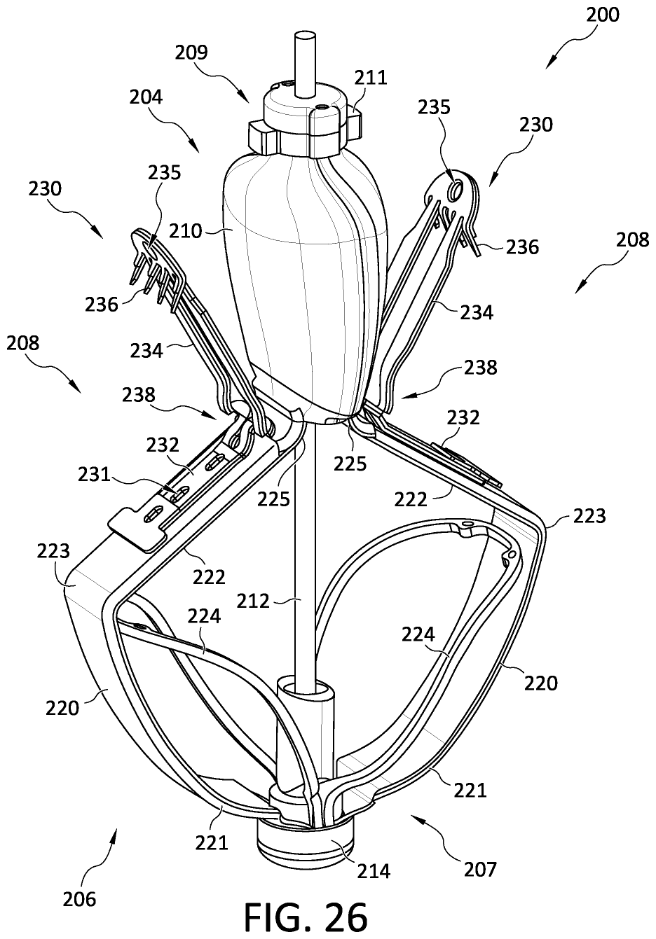

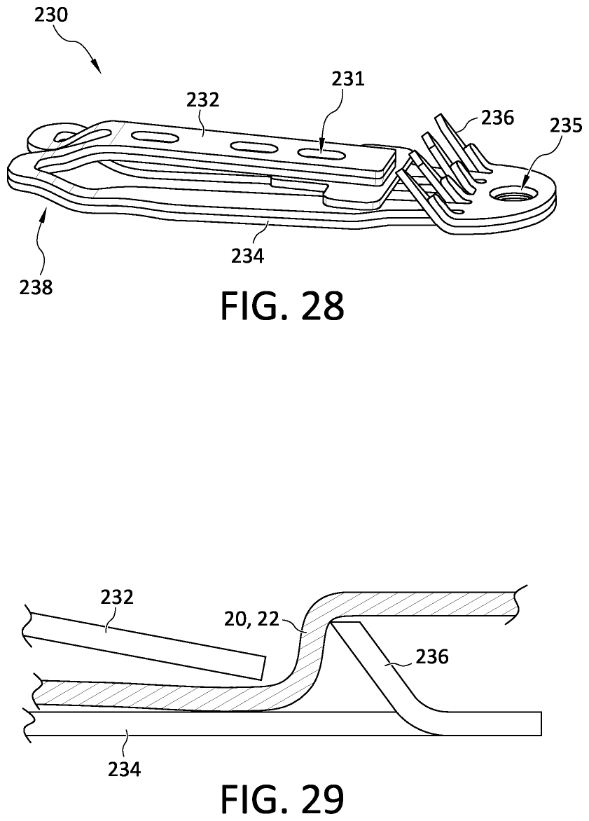

During implantation, the paddles 220, 222 of the anchors 208 are opened and closed to grasp the native valve leaflets 20, 22 between the paddles 220, 222 and the coaptation element 210. The anchors 208 are moved between a closed position (FIGS. 22-25) to various open positions (FIGS. 26-37) by extending and retracting the actuation element 212. Extending and retracting the actuation element 212 increases and decreases the spacing between the coaptation element 210 and the cap 214, respectively. The proximal collar 211 (or other attachment element) and the coaptation element 210 slide along the actuation element 212 during actuation so that changing of the spacing between the coaptation element 210 and the cap 214 causes the paddles 220, 220 to move between different positions to grasp the mitral valve leaflets 20, 22 during implantation.

As the device 200 is opened and closed, the pair of inner and outer paddles 222, 220 are moved in unison, rather than independently, by a single actuation element 212. Also, the positions of the clasps 230 are dependent on the positions of the paddles 222, 220. For example, the clasps 230 are arranged such that closure of the anchors 208 simultaneously closes the clasps 230. In some implementations, the device 200 can be made to have the paddles 220, 222 be independently controllable in the same manner (e.g., the device 101 illustrated in FIG. 15).

In some implementations, the clasps 230 further secure the native leaflets 20, 22 by engaging the leaflets 20, 22 with optional barbs and/or other friction-enhancing elements 236 and/or pinching the leaflets 20, 22 between the movable and fixed arms 234, 232. In some implementations, the clasps 230 are barbed clasps that include barbs that increase friction with and/or can partially or completely puncture the leaflets 20, 22. The actuation lines 216 (FIGS. 43-48) can be actuated separately so that each clasp 230 can be opened and closed separately. Separate operation allows one leaflet 20, 22 to be grasped at a time, or for the repositioning of a clasp 230 on a leaflet 20, 22 that was insufficiently grasped, without altering a successful grasp on the other leaflet 20, 22. The clasps 230 can be fully opened and closed when the inner paddle 222 is not closed, thereby allowing leaflets 20, 22 to be grasped in a variety of positions as the particular situation requires.

Referring now to FIGS. 22-25, the device 200 is shown in a closed position. When closed, the inner paddles 222 are disposed between the outer paddles 220 and the coaptation element 210. The clasps 230 are disposed between the inner paddles 222 and the coaptation element 210. Upon successful capture of native leaflets 20, 22 the device 200 is moved to and retained in the closed position so that the leaflets 20, 22 are secured within the device 200 by the clasps 230 and are pressed against the coaptation element 210 by the paddles 220, 222. The outer paddles 220 can have a wide curved shape that fits around the curved shape of the coaptation element 210 to more securely grip the leaflets 20, 22 when the device 200 is closed (e.g., as can be seen in FIG. 51). The curved shape and rounded edges of the outer paddle 220 also prohibits or inhibits tearing of the leaflet tissue.

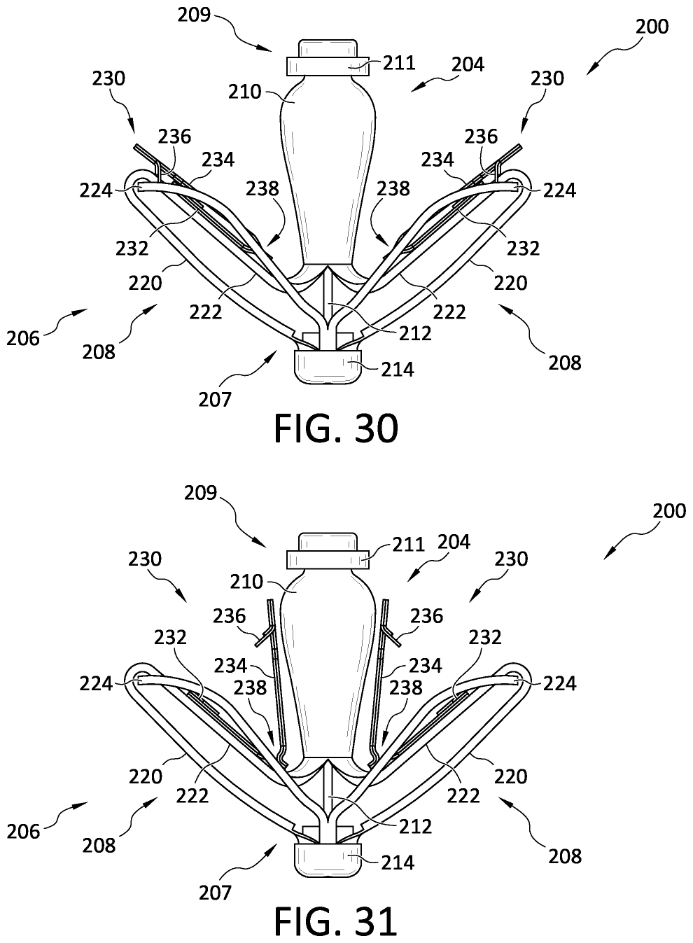

Referring now to FIGS. 30-37, the implantable device or implant 200 described above is shown in various positions and configurations ranging from partially open to fully open. The paddles 220, 222 of the device 200 transition between each of the positions shown in FIGS. 30-37 from the closed position shown in FIGS. 22-25 up extension of the actuation element 212 from a fully retracted to fully extended position.

Referring now to FIGS. 30-31, the device 200 is shown in a partially open position. The device 200 is moved into the partially open position by extending the actuation element 212. Extending the actuation element 212 pulls down on the bottom portions of the outer paddles 220 and paddle frames 224. The outer paddles 220 and paddle frames 224 pull down on the inner paddles 222, where the inner paddles 222 are connected to the outer paddles 220 and the paddle frames 224. Because the proximal collar 211 (or other attachment element) and coaptation element 210 are held in place by the capture mechanism 213, the inner paddles 222 are caused to articulate, pivot, and/or flex in an opening direction. The inner paddles 222, the outer paddles 220, and the paddle frames all flex to the position shown in FIGS. 30-31. Opening the paddles 222, 220 and frames 224 forms a gap between the coaptation element 210 and the inner paddle 222 that can receive and grasp the native leaflets 20, 22. This movement also exposes the clasps 230 that can be moved between closed (FIG. 30) and open (FIG. 31) positions to form a second gap for grasping the native leaflets 20, 22. The extent of the gap between the fixed and movable arms 232, 234 of the clasp 230 is limited to the extent that the inner paddle 222 has spread away from the coaptation element 210.

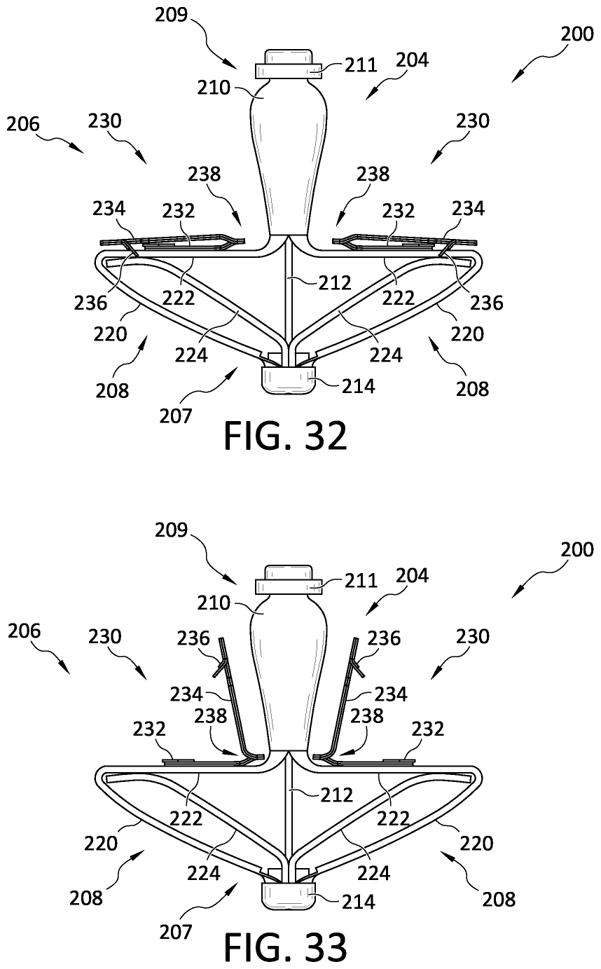

Referring now to FIGS. 32-33, the device 200 is shown in a laterally extended or open position. The device 200 is moved into the laterally extended or open position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the laterally extended or open position, the inner paddles 222 extend horizontally more than in other positions of the device 200 and form an approximately 90-degree angle with the coaptation element 210. Similarly, the paddle frames 224 are at their maximum spread position when the device 200 is in the laterally extended or open position. The increased gap between the coaptation element 210 and inner paddle 222 formed in the laterally extended or open position allows clasps 230 to open further (FIG. 33) before engaging the coaptation element 210, thereby increasing the size of the gap between the fixed and movable arms 232, 234.

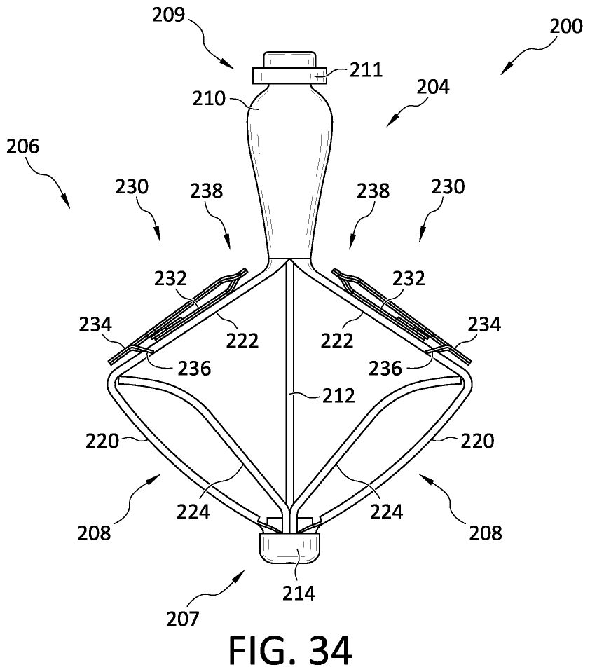

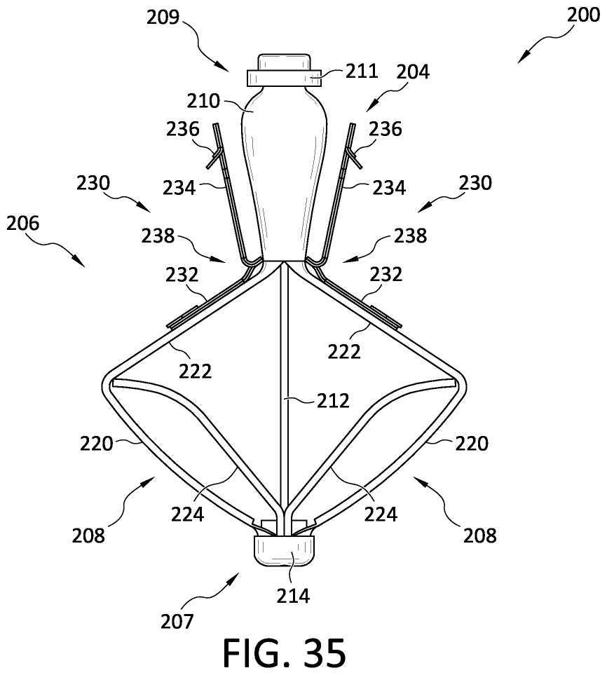

Referring now to FIGS. 34-35, the example device 200 is shown in a three-quarters extended position. The device 200 is moved into the three-quarters extended position by continuing to extend the actuation element 212 described above, thereby increasing the distance between the coaptation element 210 and the cap 214 of the distal portion 207. Continuing to extend the actuation element 212 pulls down on the outer paddles 220 and paddle frames 224, thereby causing the inner paddles 222 to spread apart further from the coaptation element 210. In the three-quarters extended position, the inner paddles 222 are open beyond 90 degrees to an approximately 135-degree angle with the coaptation element 210. The paddle frames 224 are less spread than in the laterally extended or open position and begin to move inward toward the actuation element 212 as the actuation element 212 extends further. The outer paddles 220 also flex back toward the actuation element 212. As with the laterally extended or open position, the increased gap between the coaptation element 210 and inner paddle 222 formed in the laterally extended or open position allows clasps 230 to open even further (FIG. 35), thereby increasing the size of the gap between the fixed and movable arms 232, 234.

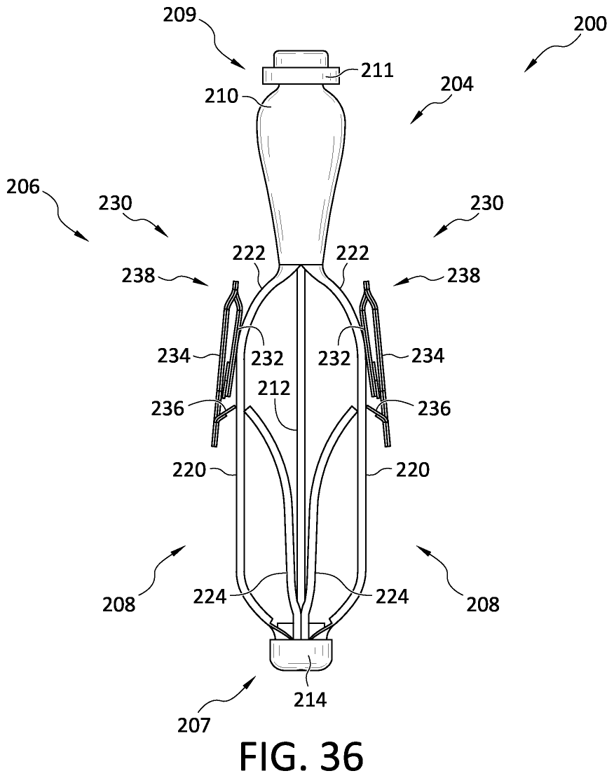

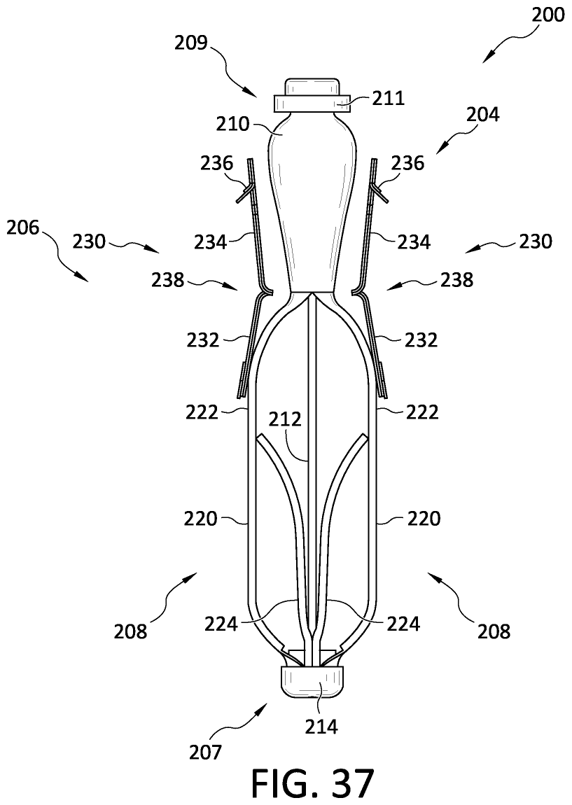

Referring now to FIGS. 36-37, the example device 200 is shown in a fully extended position. The device 200 is moved into the fully extended position by continuing to extend the actuation element 212 described above, thereby increasing the distance between the coaptation element 210 and the cap 214 of the distal portion 207 to a maximum distance allowable by the device 200. Continuing to extend the actuation element 212 pulls down on the outer paddles 220 and paddle frames 224, thereby causing the inner paddles 222 to spread apart further from the coaptation element 210. The outer paddles 220 and paddle frames 224 move to a position where they are close to the actuation element. In the fully extended position, the inner paddles 222 are open to an approximately 180-degree angle with the coaptation element 210. The inner and outer paddles 222, 220 are stretched straight in the fully extended position to form an approximately 180-degree angle between the paddles 222, 220. The fully extended position of the device 200 provides the maximum size of the gap between the coaptation element 210 and inner paddle 222, and, in some implementations, allows clasps 230 to also open fully to approximately 180 degrees (FIG. 37) between the fixed and movable arms 232, 234 of the clasp 230. The position of the device 200 is the longest and the narrowest configuration. Thus, the fully extended position of the device 200 can be a desirable position for bailout of the device 200 from an attempted implantation or can be a desired position for placement of the device in a delivery catheter, or the like.

Configuring the device or implant 200 such that the anchors 208 can extend to a straight or approximately straight configuration (e.g., approximately 120-180 degrees relative to the coaptation element 210) can provide several advantages. For example, this configuration can reduce the radial crimp profile of the device or implant 200. It can also make it easier to grasp the native leaflets 20, 22 by providing a larger opening between the coaptation element 210 and the inner paddles 222 in which to grasp the native leaflets 20, 22. Additionally, the relatively narrow, straight configuration can prevent or reduce the likelihood that the device or implant 200 will become entangled in native anatomy (e.g., chordae tendineae CT shown in FIGS. 3 and 4) when positioning and/or retrieving the device or implant 200 into the delivery system 202.

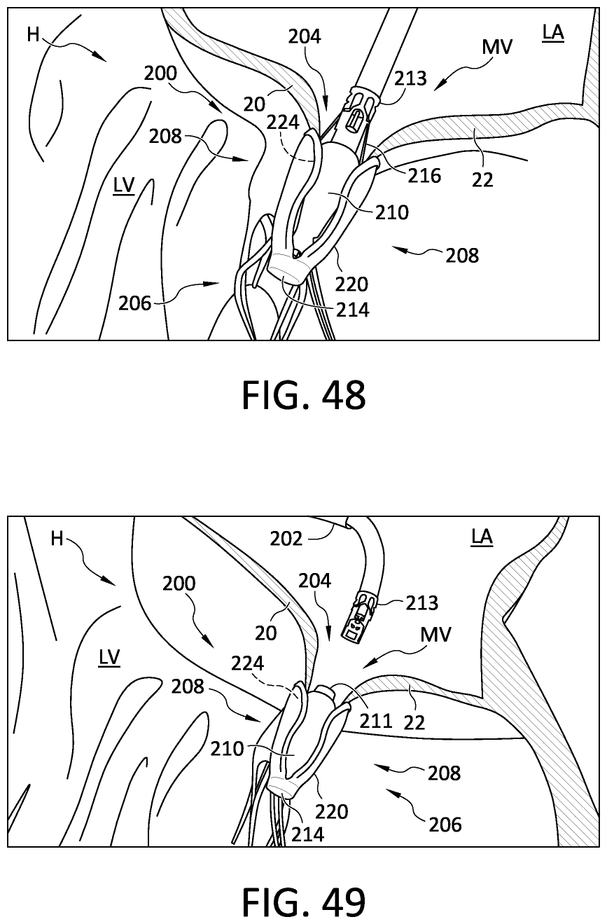

Referring now to FIGS. 38-49, an example device 200 is shown being delivered and deployed within the native mitral valve MV of the heart H. As described above, the device 200 shown in FIGS. 38-49 includes the optional covering 240 (e.g., FIG. 25) over the coaptation element 210, clasps 230, inner paddles 222 and/or the outer paddles 220. The device 200 is deployed from a delivery system 202 (e.g., which can comprise an implant catheter that is extendable from a steerable catheter 241 and/or a guide sheath) and is retained by a capture mechanism 213 (see e.g., FIGS. 43 and 48) and is actuated by extending or retracting the actuation element 212. Fingers of the capture mechanism 213 removably attach the collar 211 to the delivery system 202. In some implementations, the capture mechanism 213 is held closed around the collar 211 by the actuation element 212, such that removal of the actuation element 212 allows the fingers of the capture mechanism 213 to open and release the collar 211 to decouple the capture mechanism 213 from the device 200 after the device 200 has been successfully implanted.

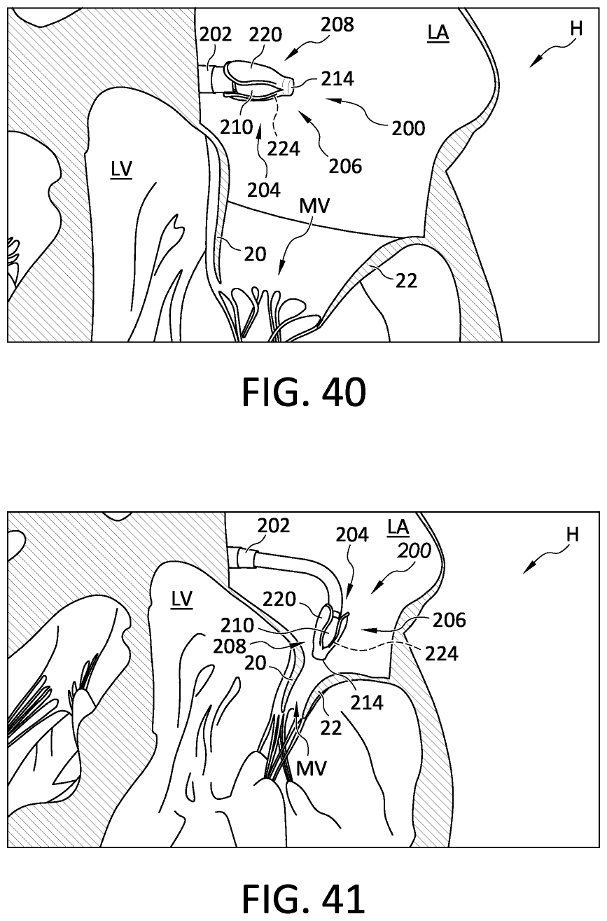

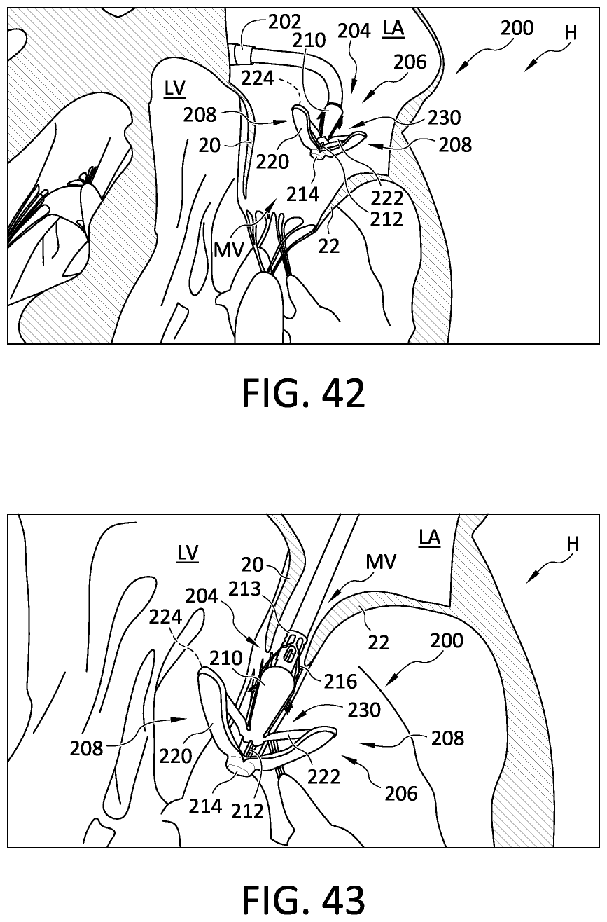

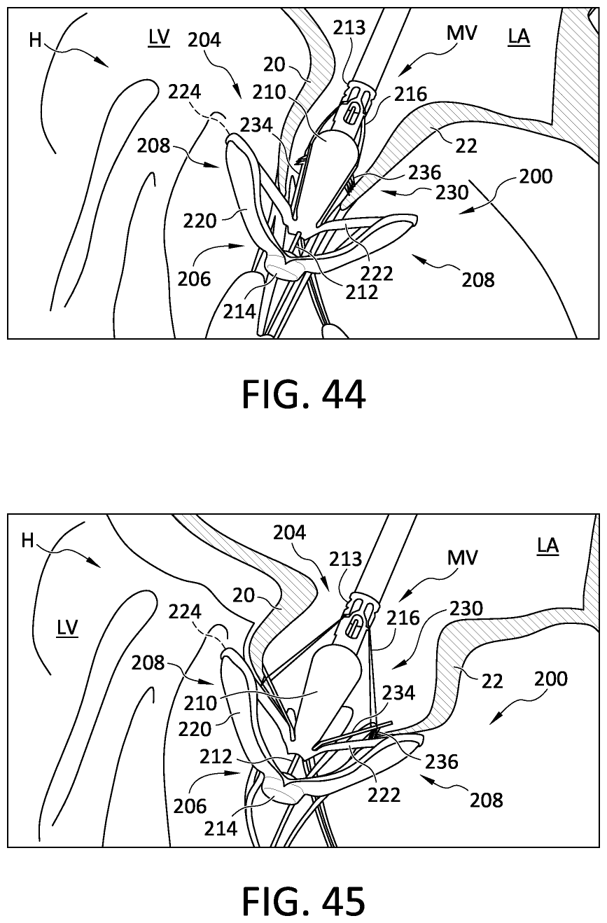

Referring now to FIG. 38, the delivery system 202 (e.g., a delivery catheter/sheath thereof) is inserted into the left atrium LA through the septum and the device/implant 200 is deployed from the delivery system 202 (e.g., an implant catheter retaining the device/implant can be extended to deploy the device/implant out from a steerable catheter) in the fully open condition for the reasons discussed above with respect to the device 100. The actuation element 212 is then retracted to move the device 200 through the partially closed condition (FIG. 39) and to the fully closed condition shown in FIGS. 40-41. Then the delivery system or catheter maneuvers the device/implant 200 towards the mitral valve MV as shown in FIG. 41. Referring now to FIG. 42, when the device 200 is aligned with the mitral valve MV, the actuation element 212 is extended to open the paddles 220, 222 into the partially opened position and the actuation lines 216 (FIGS. 43-48) are retracted to open the clasps 230 to prepare for leaflet grasp. Next, as shown in FIGS. 43-44, the partially open device 200 is inserted through the native valve (e.g., by advancing an implant catheter from a steerable catheter) until leaflets 20, 22 are properly positioned in between the inner paddles 222 and the coaptation element 210 and inside the open clasps 230.

FIG. 45 shows the device 200 with both clasps 230 closed, though the optional barbs 236 of one clasp 230 missed one leaflet 22. As can be seen in FIGS. 45-47, the out of position clasp 230 is opened and closed again to properly grasp the missed leaflet 22. When both leaflets 20, 22 are grasped properly, the actuation element 212 is retracted to move the device 200 into the fully closed position shown in FIG. 48. With the device 200 fully closed and implanted in the native valve, the actuation element 212 is disengaged from the cap 214 and is withdrawn to release the capture mechanism 213 from the proximal collar 211 (or other attachment element) so that the capture mechanism 213 can be withdrawn into the delivery system 202 (e.g., into a catheter/sheath), as shown in FIG. 49. Once deployed, the device 200 can be maintained in the fully closed position with a mechanical means such as a latch or can be biased to remain closed through the use of spring material, such as steel, and/or shape-memory alloys such as Nitinol. For example, the paddles 220, 222 can be formed of steel or Nitinol shape-memory alloy—produced in a wire, sheet, tubing, or laser sintered powder—and are biased to hold the outer paddles 220 closed around the inner paddles 222, coaptation element 210, and/or the clasps 230 pinched around native leaflets 20, 22.

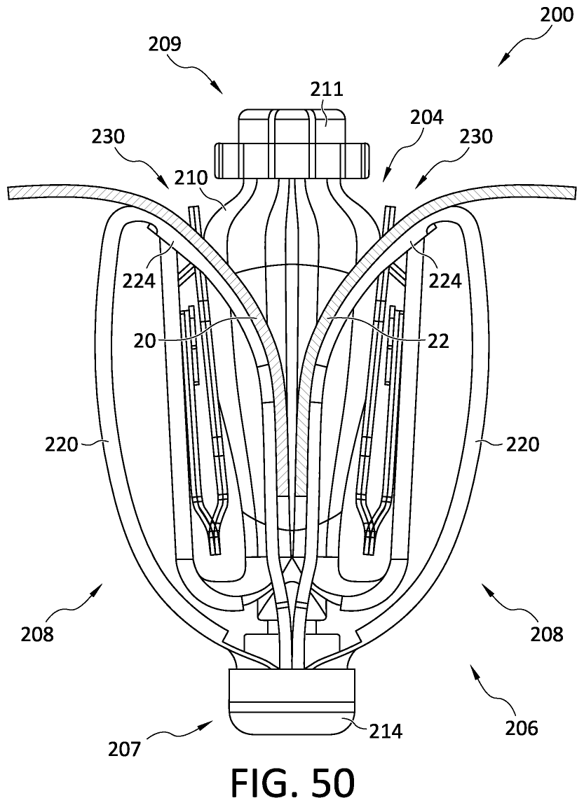

Referring to FIGS. 50-54, once the device 200 is implanted in a native valve, the coaptation element 210 functions as a gap filler in the valve regurgitant orifice, such as the gap 26 in the mitral valve MV illustrated by FIG. 6 or a gap in another native valve. In some implementations, when the device 200 has been deployed between the two opposing valve leaflets 20, 22, the leaflets 20, 22 no longer coapt against each other in the area of the coaptation element 210, but instead coapt against the coaptation element 210. This reduces the distance the leaflets 20, 22 need to be approximated to close the mitral valve MV during systole, thereby facilitating repair of functional valve disease that may be causing mitral regurgitation. A reduction in leaflet approximation distance can result in several other advantages as well. For example, the reduced approximation distance required of the leaflets 20, 22 reduces or minimizes the stress experienced by the native valve. Shorter approximation distance of the valve leaflets 20,22 can also require less approximation forces which can result in less tension experienced by the leaflets 20, 22 and less diameter reduction of the valve annulus. The smaller reduction of the valve annulus—or none at all—can result in less reduction in valve orifice area as compared to a device without a coaptation element or spacer. In this way, the coaptation element 210 can reduce the transvalvular gradients.

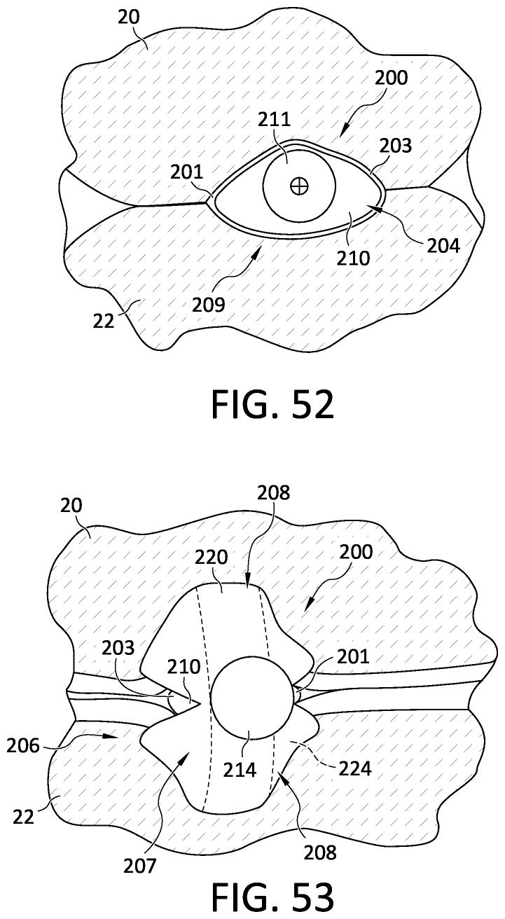

To adequately fill the gap 26 between the leaflets 20, 22, the device 200 and the components thereof can have a wide variety of different shapes and sizes. For example, the outer paddles 220 and paddle frames 224 can be configured to conform to the shape or geometry of the coaptation element 210 as is shown in FIGS. 50-54. As a result, the outer paddles 220 and paddle frames 224 can mate with both the coaptation element 210 and the native valve leaflets 20, 22. In some implementations, when the leaflets 20, 22 are coapted against the coaptation element 210, the leaflets 20, 22 fully surround or “hug” the coaptation element 210 in its entirety, thus small leaks at lateral and medial aspects 201, 203 of the coaptation element 210 can be prevented or inhibited. The interaction of the leaflets 20, 22 and the device 200 is made clear in FIG. 51, which shows a schematic atrial or surgeon's view that shows the paddle frame 224 (which would not actually be visible from a true atrial view, e.g., FIG. 52), conforming to the coaptation element 210 geometry. The opposing leaflets 20, 22 (the ends of which would also not be visible in the true atrial view, e.g., FIG. 52) being approximated by the paddle frames 224, to fully surround or “hug” the coaptation element 210.

This coaptation of the leaflets 20, 22 against the lateral and medial aspects 201, 203 of the coaptation element 210 (shown from the atrial side in FIG. 52, and the ventricular side in FIG. 53) would seem to contradict the statement above that the presence of a coaptation element 210 minimizes the distance the leaflets need to be approximated. However, the distance the leaflets 20, 22 need to be approximated is still minimized if the coaptation element 210 is placed precisely at a regurgitant gap 26 and the regurgitant gap 26 is less than the width (medial—lateral) of the coaptation element 210.

FIG. 50 illustrates the geometry of the coaptation element 210 and the paddle frame 224 from an LVOT perspective. As can be seen in this view, the coaptation element 210 has a tapered shape being smaller in dimension in the area closer to where the inside surfaces of the leaflets 20, 22 are required to coapt and increase in dimension as the coaptation element 210 extends toward the atrium. Thus, the depicted native valve geometry is accommodated by a tapered coaptation element geometry. Still referring to FIG. 50, the tapered coaptation element geometry, in conjunction with the illustrated expanding paddle frame shape (toward the valve annulus) can help to achieve coaptation on the lower end of the leaflets, reduce stress, and minimize transvalvular gradients.

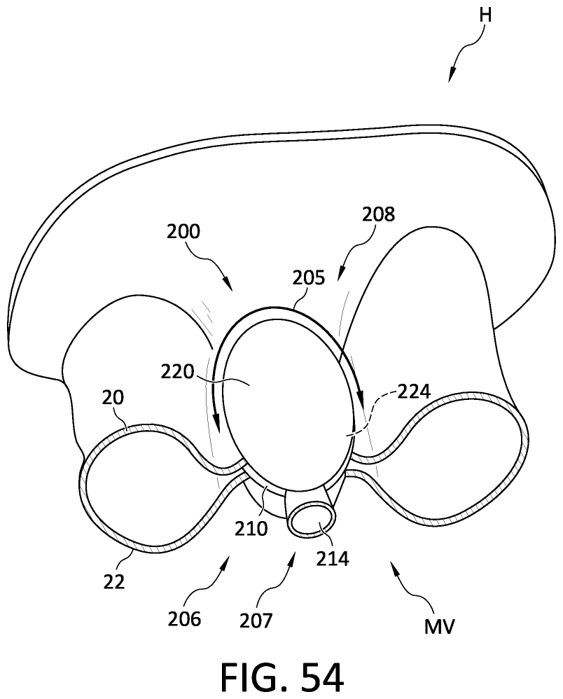

Referring to FIG. 54, the shape of the coaptation element 210 and the paddle frames 224 can be defined based on an Intra-Commissural view of the native valve and the device 200. Two factors of these shapes are leaflet coaptation against the coaptation element 210 and reduction of stress on the leaflets due to the coaptation. Referring to FIGS. 54 and 24, to both coapt the valve leaflets 20, 22 against the coaptation element 210 and reduce the stress applied to the valve leaflets 20, 22 by the coaptation element 210 and/or the paddle frames 224, the coaptation element 210 can have a round or rounded shape and the paddle frames 224 can have a full radius that spans nearly the entirety of the paddle frame 224. The round shape of the coaptation element 210 and/or the illustrated fully rounded shape of the paddle frames 224 distributes the stresses on the leaflets 20, 22 across a large, curved engagement area 209. For example, in FIG. 54, the force on the leaflets 20, 22 by the paddle frames is spread along the entire rounded length of the paddle frame 224, as the leaflets 20 try to open during the diastole cycle.

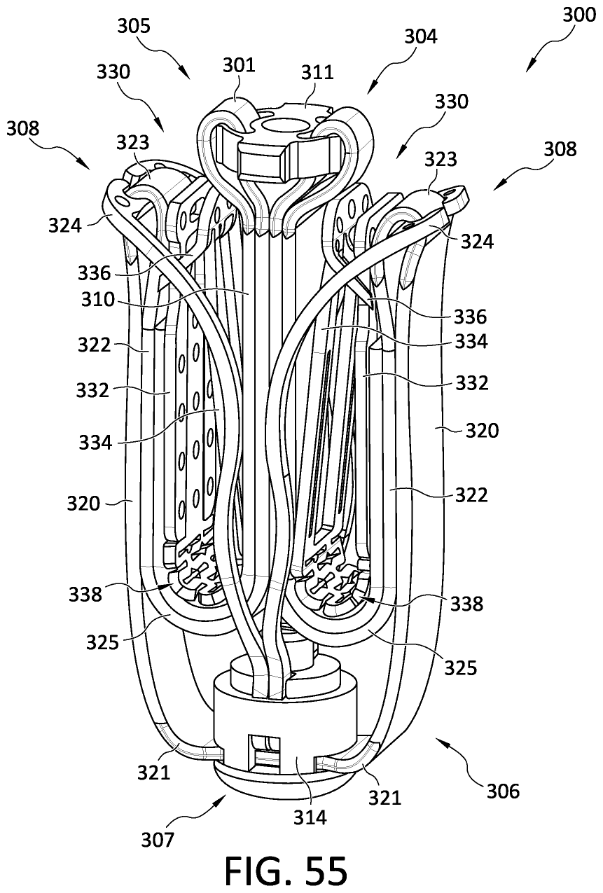

Referring now to FIG. 55, an example of an implantable device or implant 300 is shown. The implantable device 300 is one of the many different configurations that the device 100 that is schematically illustrated in FIGS. 8-14 can take. The device 300 can include any other features for an implantable device or implant discussed in the present application, and the device 300 can be positioned to engage valve tissue 20, 22 as part of any suitable valve repair system (e.g., any valve repair system disclosed in the present application).

The implantable device or implant 300 includes a proximal or attachment portion 305, an anchor portion 306, and a distal portion 307. In some implementations, the device/implant 300 includes a coaptation portion 304, and the coaptation portion 304 can optionally include a coaptation element 310 (e.g., spacer, plug, membrane, sheet, etc.) for implantation between the leaflets 20, 22 of the native valve. In some implementations, the anchor portion 306 includes a plurality of anchors 308. In some implementations, each anchor 308 can include one or more paddles, e.g., outer paddles 320, inner paddles 322, paddle extension members or paddle frames 324. The anchors can also include and/or be coupled to clasps 330. In some implementations, the attachment portion 305 includes a first or proximal collar 311 (or other attachment element) for engaging with a capture mechanism (e.g., a capture mechanism such as the capture mechanism 213 shown in FIGS. 43-49, or another capture mechanism described herein or otherwise known) of a delivery system (e.g., a delivery system such as the system shown in FIGS. 38-42 and 49).

The anchors 308 can be attached to the other portions of the device and/or to each other in a variety of different ways (e.g., directly, indirectly, welding, sutures, adhesive, links, latches, integrally formed, a combination of some or all of these, etc.). In some implementations, the anchors 308 are attached to a coaptation member or coaptation element 310 by connection portions 325 and to a cap 314 by connection portions 321.

The anchors 308 can comprise first portions or outer paddles 320 and second portions or inner paddles 322 separated by connection portions 323. The connection portions 323 can be attached to paddle frames 324 that are hingeably attached to a cap 314 or other attachment portion. In this manner, the anchors 308 are configured similar to legs in that the inner paddles 322 are like upper portions of the legs, the outer paddles 320 are like lower portions of the legs, and the connection portions 323 are like knee portions of the legs.

In implementations with a coaptation member or coaptation element 310, the coaptation member or coaptation element 310 and the anchors 308 can be coupled together in various ways. For example, as shown in the illustrated example, the coaptation element 310 and the anchors 308 can be coupled together by integrally forming the coaptation element 310 and the anchors 308 as a single, unitary component. This can be accomplished, for example, by forming the coaptation element 310 and the anchors 308 from a continuous strip 301 of a braided or woven material, such as braided or woven nitinol wire. In the illustrated example, the coaptation element 310, the outer paddle portions 320, the inner paddle portions 322, and the connection portions 321, 323, 325 are formed from the continuous strip of fabric 301.

Like the anchors 208 of the implantable device or implant 200 described above, the anchors 308 can be configured to move between various configurations by axially moving the distal end of the device (e.g., cap 314, etc.) relative to the proximal end of the device (e.g., proximal collar 311 or other attachment element, etc.) and thus the anchors 308 move relative to a midpoint of the device. This movement can be along a longitudinal axis extending between the distal end (e.g., cap 314, etc.) and the proximal end (e.g., collar 311 or other attachment element, etc.) of the device. For example, the anchors 308 can be positioned in a fully extended or straight configuration (e.g., similar to the configuration of device 200 shown in FIG. 36) by moving the distal end (e.g., cap 314, etc.) away from the proximal end of the device.

In some implementations, in the straight configuration, the paddle portions 320, 322 are aligned or straight in the direction of the longitudinal axis of the device. In some implementations, the connection portions 323 of the anchors 308 are adjacent the longitudinal axis of the coaptation element 310 (e.g., similar to the configuration of device 200 shown in FIG. 36). From the straight configuration, the anchors 308 can be moved to a fully folded configuration (e.g., FIG. 55), e.g., by moving the proximal end and distal end toward each other and/or toward a midpoint or center of the device. Initially, as the distal end (e.g., cap 314, etc.) moves toward the proximal end and/or midpoint or center of the device, the anchors 308 bend at connection portions 321, 323, 325, and the connection portions 323 move radially outwardly relative to the longitudinal axis of the device 300 and axially toward the midpoint and/or toward the proximal end of the device (e.g., similar to the configuration of device 200 shown in FIG. 34). As the cap 314 continues to move toward the midpoint and/or toward the proximal end of the device, the connection portions 323 move radially inwardly relative to the longitudinal axis of the device 300 and axially toward the proximal end of the device (e.g., similar to the configuration of device 200 shown in FIG. 30).

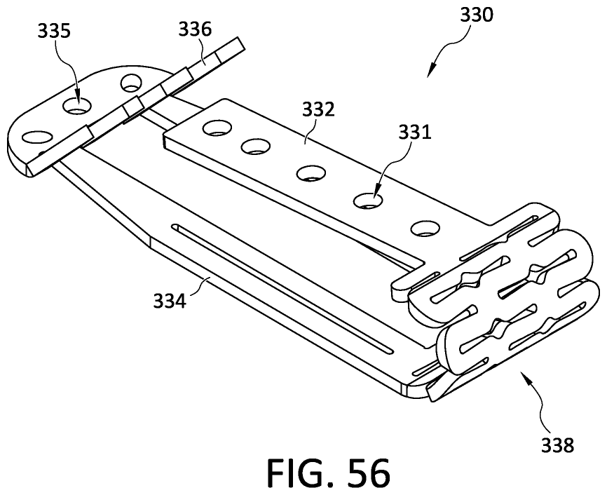

In some implementations, the clasps comprise a movable arm coupled to an anchor. In some implementations, the clasps 330 (as shown in detail in FIG. 56) include a base or fixed arm 332, a movable arm 334, optional barbs/friction-enhancing elements 336, and a joint portion 338. The fixed arms 332 are attached to the inner paddles 322, with the joint portion 338 disposed proximate the coaptation element 310. The joint portion 338 is spring-loaded so that the fixed and movable arms 332, 334 are biased toward each other when the clasp 330 is in a closed condition.

The fixed arms 332 are attached to the inner paddles 322 through holes or slots 331 with sutures (not shown). The fixed arms 332 can be attached to the inner paddles 322 with any suitable means, such as screws or other fasteners, crimped sleeves, mechanical latches or snaps, welding, adhesive, or the like. The fixed arms 332 remain substantially stationary relative to the inner paddles 322 when the movable arms 334 are opened to open the clasps 330 and expose the optional barbs 336. The clasps 330 are opened by applying tension to actuation lines (e.g., the actuation lines 216 shown in FIGS. 43-48) attached to holes 335 in the movable arms 334, thereby causing the movable arms 334 to articulate, pivot, and/or flex on the joint portions 338.

In short, the implantable device or implant 300 is similar in configuration and operation to the implantable device or implant 200 described above, except that the coaptation element 310, outer paddles 320, inner paddles 322, and connection portions 321, 323, 325 are formed from the single strip of material 301. In some implementations, the strip of material 301 is attached to the proximal collar 311, cap 314, and paddle frames 324 by being woven or inserted through openings in the proximal collar 311, cap 314, and paddle frames 324 that are configured to receive the continuous strip of material 301. The continuous strip 301 can be a single layer of material or can include two or more layers. In some implementations, portions of the device 300 have a single layer of the strip of material 301 and other portions are formed from multiple overlapping or overlying layers of the strip of material 301.

For example, FIG. 55 shows a coaptation element 310 and inner paddles 322 formed from multiple overlapping layers of the strip of material 301. The single continuous strip of material 301 can start and end in various locations of the device 300. The ends of the strip of material 301 can be in the same location or different locations of the device 300. For example, in the illustrated example of FIG. 55, the strip of material 301 begins and ends in the location of the inner paddles 322.

As with the implantable device or implant 200 described above, the size of the coaptation element 310 can be selected to minimize the number of implants that a single patient will require (preferably one), while at the same time maintaining low transvalvular gradients. In particular, forming many components of the device 300 from the strip of material 301 allows the device 300 to be made smaller than the device 200. For example, in some implementations, the anterior-posterior distance at the top of the coaptation element 310 is less than 2 mm, and the medial-lateral distance of the device 300 (i.e., the width of the paddle frames 324 which are wider than the coaptation element 310) at its widest is about 5 mm.

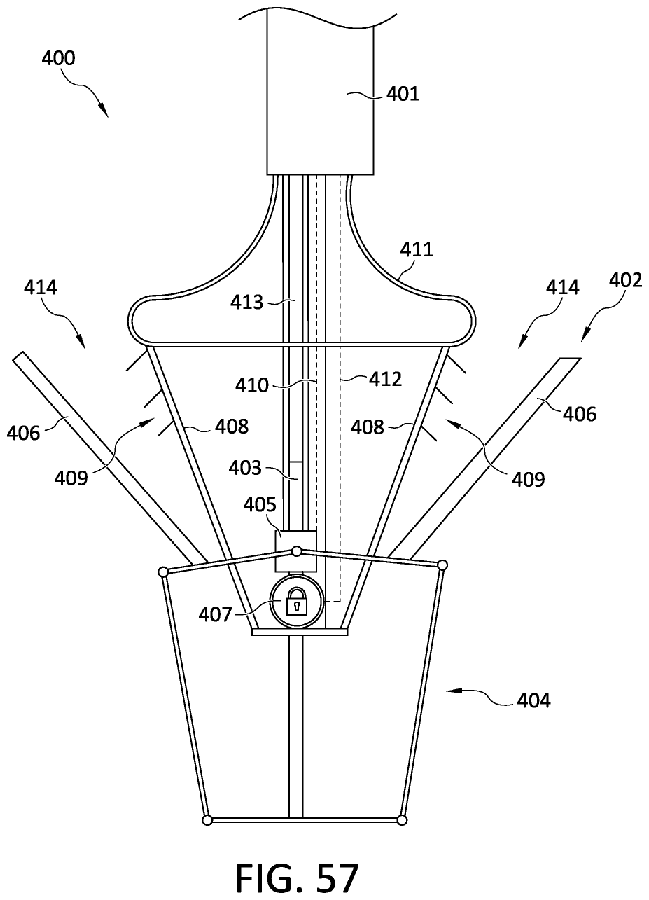

FIGS. 57-63 illustrate another example of one of the many valve repair systems 400 for repairing a native valve of a patient that the concepts of the present application can be applied to. The valve repair system 400 includes a delivery device 401 and a valve repair device 402.

The valve repair device 402 includes a base assembly 404, a pair of paddles 406, and a pair of gripping members 408 (e.g., clasps, clasp arms, grippers, gripping arms, latches, etc.). In some implementations, the paddles 406 can be integrally formed with the base assembly. For example, the paddles 406 can be formed as extensions of links of the base assembly. In the illustrated example, the base assembly 404 of the valve repair device 402 has a shaft 403, a coupler 405 configured to move along the shaft, and a lock 407 configured to lock the coupler in a stationary position on the shaft. The coupler 405 is mechanically connected to the paddles 406, such that movement of the coupler 405 along the shaft 403 causes the paddles to move between an open position and a closed position. In this way, the coupler 405 serves as a means for mechanically coupling the paddles 406 to the shaft 403 and, when moving along the shaft 403, for causing the paddles 406 to move between their open and closed positions.

In some implementations, the gripping members 408 are pivotally connected to the base assembly 404 (e.g., the gripping members 408 can be pivotally connected to the shaft 403, or any other suitable member of the base assembly), such that the gripping members can be moved to adjust the width of the opening 414 between the paddles 406 and the gripping members 408. The gripping member 408 can include an optional barbed portion 409 for attaching the gripping members to valve tissue when the valve repair device 402 is attached to the valve tissue. The gripping member 408 forms a means for gripping the valve tissue (in particular tissue of the valve leaflets) with a sticking means or portion such as the optional barbed portion 409. When the paddles 406 are in the closed position, the paddles engage the gripping members 408, such that, when valve tissue is attached to the optional barbed portion 409 of the gripping members, the paddles act as holding or securing means to hold the valve tissue at the gripping members and to secure the valve repair device 402 to the valve tissue. In some implementations, the gripping members 408 are configured to engage the paddles 406 such that the optional barbed portion 409 engages the valve tissue member and the paddles 406 to secure the valve repair device 402 to the valve tissue member. For example, in certain situations, it can be advantageous to have the paddles 406 maintain an open position and have the gripping members 408 move outward toward the paddles 406 to engage valve tissue and the paddles 406.

While the examples shown in FIGS. 57-63 illustrate a pair of paddles 406 and a pair of gripping members 408, it should be understood that the valve repair device 402 can include any suitable number of paddles and gripping members.

In some implementations, the valve repair system 400 includes a placement shaft 413 that is removably attached to the shaft 403 of the base assembly 404 of the valve repair device 402. After the valve repair device 402 is secured to valve tissue, the placement shaft 413 is removed from the shaft 403 to remove the valve repair device 402 from the remainder of the valve repair system 400, such that the valve repair device 402 can remain attached to the valve tissue, and the delivery device 401 can be removed from a patient's body.

The valve repair system 400 can also include a paddle control mechanism 410, a gripper control mechanism 411, and a lock control mechanism 412. The paddle control mechanism 410 is mechanically attached to the coupler 405 to move the coupler along the shaft, which causes the paddles 406 to move between the open and closed positions. The paddle control mechanism 410 can take any suitable form, and can comprise, for example, a shaft, wire, tube, hypotube, rod, suture, line, etc. For example, the paddle control mechanism can comprise a hollow shaft, a catheter tube or a sleeve that fits over the placement shaft 413 and the shaft 403 and is connected to the coupler 405.

The gripper control mechanism 411 is configured to move the gripping members 408 such that the width of the opening 414 between the gripping members and the paddles 406 can be altered. The gripper control mechanism 411 can take any suitable form, such as, for example, a line, a suture, a wire, a rod, a catheter, a tube, a hypotube, etc.

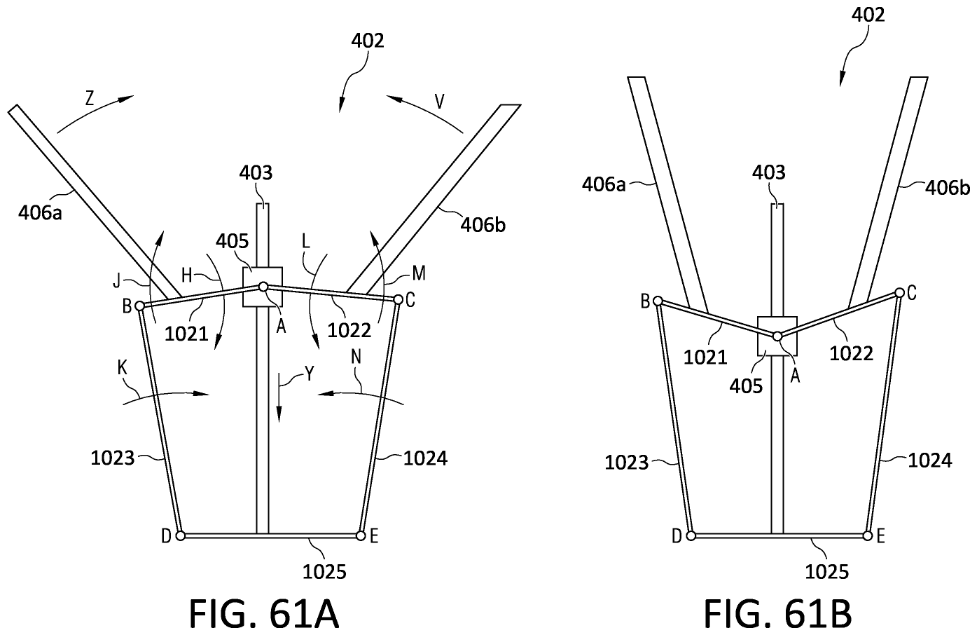

The lock control mechanism 412 is configured to lock and unlock the lock. The lock 407 serves as a locking means for locking the coupler 405 in a stationary position with respect to the shaft 403 and can take a wide variety of different forms and the type of lock control mechanism 412 can be dictated by the type of lock used. In some implementations, the lock 407 includes a pivotable plate having a hole, in which the shaft 403 of the valve repair device 402 is disposed within the hole of the pivotable plate. In this example, when the pivotable plate is in the tilted position, the pivotable plate engages the shaft 403 to maintain a position on the shaft 403, but, when the pivotable plate is in a substantially non-tilted position, the pivotable plate can be moved along the shaft (which allows the coupler 405 to move along the shaft 403). In other words, the coupler 405 is prevented or inhibited from moving in the direction Y (as shown in FIG. 61A) along the shaft 403 when the pivotable plate of the lock 407 is in a tilted (or locked) position, and the coupler is allowed to move in the direction Y along the shaft 403 when the pivotable plate is in a substantially non-tilted (or unlocked) position. In examples in which the lock 407 includes a pivotable plate, the lock control mechanism 412 is configured to engage the pivotable plate to move the plate between the tilted and substantially non-tilted positions. The lock control mechanism 412 can be, for example, a rod, a suture, a wire, or any other member that is capable of moving a pivotable plate of the lock 407 between a tilted and substantially non-tilted position. In some implementations, the pivotable plate of the lock 407 is biased in the tilted (or locked) position, and the lock control mechanism 412 is used to move the plate from the tilted position to the substantially non-tilted (or unlocked) position. In some implementations, the pivotable plate of the lock 407 is biased in the substantially non-tilted (or unlocked) position, and the lock control mechanism 412 is used to move the plate from the substantially non-tilted position to the tilted (or locked) position.

FIGS. 61A-61B illustrate the valve repair device 402 moving from an open position (as shown in FIG. 61A) to a closed position (as shown in FIG. 61B). The base assembly 404 includes a first link 1021 extending from point A to point B, a second link 1022 extending from point A to point C, a third link 1023 extending from point B to point D, a fourth link 1024 extending from point C to point E, and a fifth link 1025 extending from point D to point E. The coupler 405 is movably attached to the shaft 403, and the shaft 403 is fixed to the fifth link 1025. The first link 1021 and the second link 1022 are pivotally attached to the coupler 405 at point A, such that movement of the coupler 405 along the shaft 403 moves the location of point A and, consequently, moves the first link 1021 and the second link 1022. The first link 1021 and the third link 1023 are pivotally attached to each other at point B, and the second link 1022 and the fourth link 1024 are pivotally attached to each other at point C. One paddle 406a is attached to first link 1021 such that movement of first link 1021 causes the paddle 406a to move, and the other paddle 406b is attached to the second link 1022 such that movement of the second link 1022 causes the paddle 406b to move. In some implementations, the paddles 406a, 406b can be connected to links 1023, 1024 or be extensions of links 1023, 1024.

In order to move the valve repair device from the open position (as shown in FIG. 61A) to the closed position (as shown in FIG. 61B), the coupler 405 is moved along the shaft 403 in the direction Y, which moves the pivot point A for the first link 1021 and the second link 1022 to a new position. Movement of the coupler 405 (and pivot point A) in the direction Y causes a portion of the first link 1021 near point A to move in the direction H, and the portion of the first link 1021 near point B to move in the direction J. The paddle 406a is attached to the first link 1021 such that movement of the coupler 405 in the direction Y causes the paddle 406a to move in the direction Z. In addition, the third link 1023 is pivotally attached to the first link 1021 at point B such that movement of the coupler 405 in the direction Y causes the third link 1023 to move in the direction K. Similarly, movement of the coupler 405 (and pivot point A) in the direction Y causes a portion of the second link 1022 near point A to move in the direction L, and the portion of the second link 1022 near point C to move in the direction M. The paddle 406b is attached to the second link 1022 such that movement of the coupler 405 in the direction Y causes the paddle 406b to move in the direction V. In addition, the fourth link 1024 is pivotally attached to the second link 1022 at point C such that movement of the coupler 405 in the direction Y causes the fourth link 1024 to move in the direction N. FIG. 61B illustrates the final position of the valve repair device 402 after the coupler 405 is moved as shown in FIG. 61A.

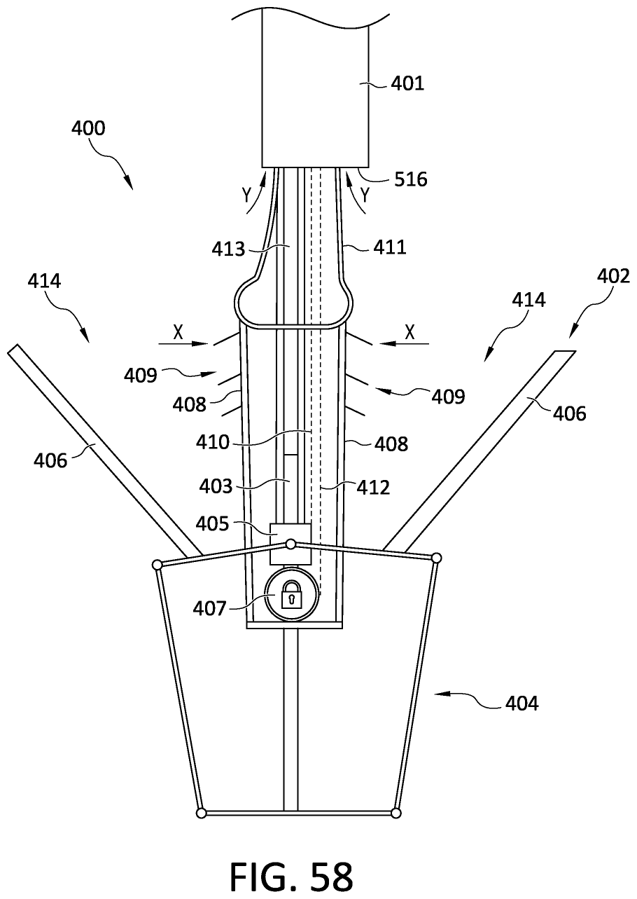

Referring to FIG. 58, the valve repair device 402 is shown in the open position (similar to the position shown in FIG. 61A), and the gripper control mechanism 411 is shown moving the gripping members 408 to provide a wider gap at the opening 414 between the gripping members and the paddles 406. In the illustrated example, the gripper control mechanism 411 includes a line, such as a suture, a wire, etc. that is threaded through an opening in an end of the gripper members 408. Both ends of the line extend through the delivery opening 516 of the delivery device 401. When the line is pulled through the delivery opening 516 in the direction Y, the gripping members 408 move inward in the direction X, which causes the opening 414 between the gripping members and the paddles 406 to become wider.

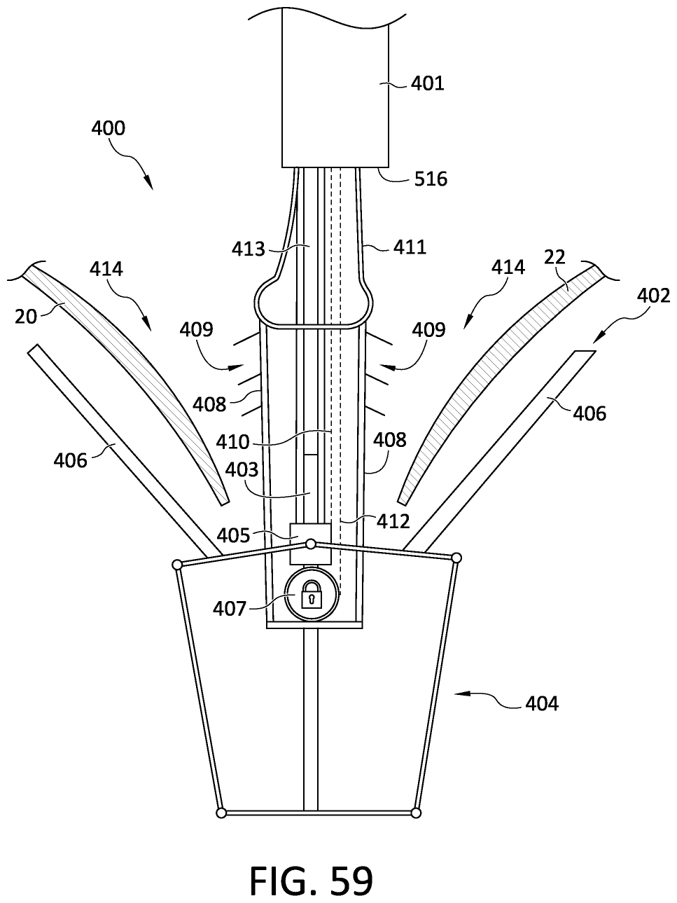

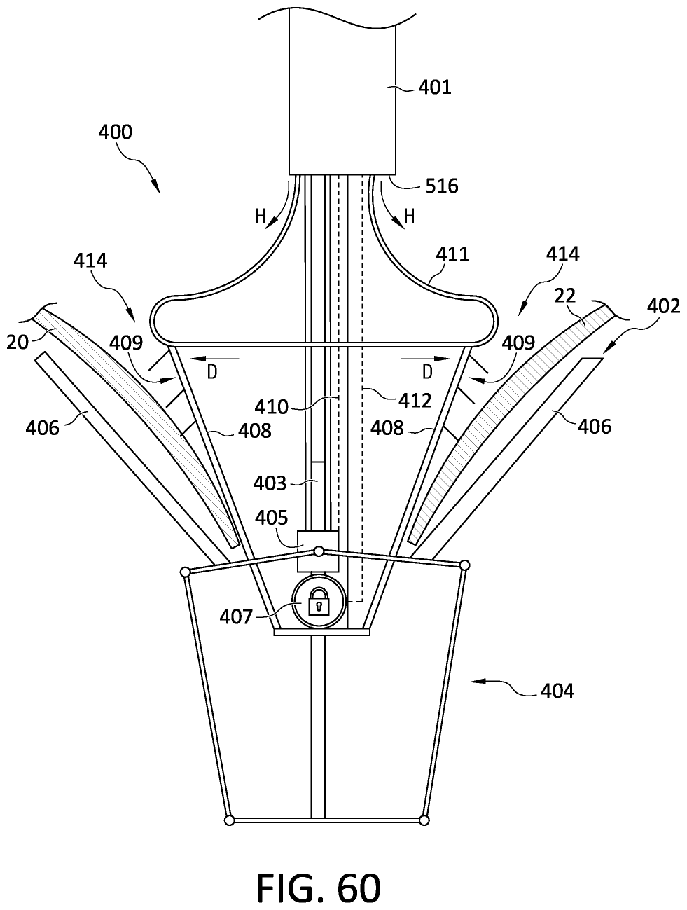

Referring to FIG. 59, the valve repair device 402 is shown such that valve tissue 20, 22 is disposed in the opening 414 between the gripping members 408 and the paddles 406. Referring to FIG. 60, after the valve tissue 20, 22 is disposed between the gripping members 408 and the paddles 406, the gripper control mechanism 411 is used to lessen the width of the opening 414 between the gripping members and the paddles. That is, in the illustrated example, the line of the gripper control mechanism 411 is released from or pushed out of the opening 516 of the delivery member in the direction H, which allows the gripping members 408 to move in the direction D to lessen the width of the opening 414. While the gripper control mechanism 411 is shown moving the gripping members 408 to increase the width of the opening 414 between the gripping members and the paddles 406 (FIG. 59), it should be understood that the gripping members may not need to be moved in order to position valve tissue in the opening 414. In certain circumstances, however, the opening 414 between the paddles 406 and the gripping members 408 can be wider in order to receive the valve tissue.

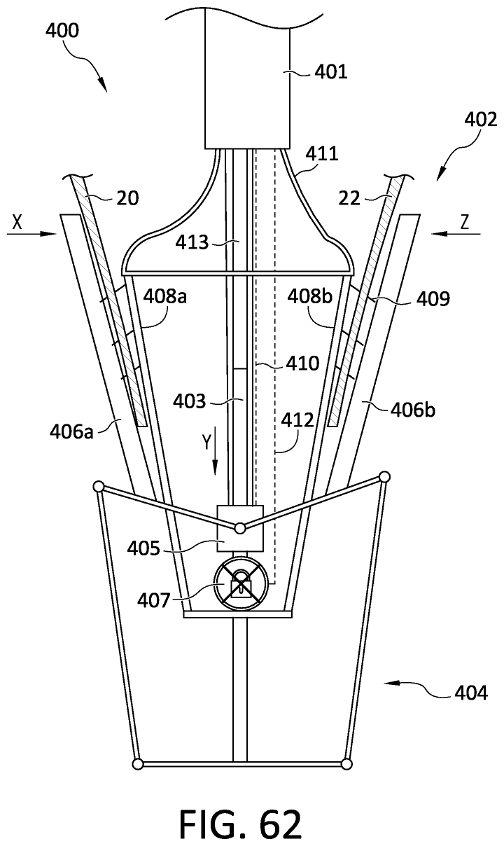

Referring to FIG. 62, the valve repair device 402 is in the closed position and secured to valve tissue 20, 22. The valve repair device 402 is secured to the valve tissue 20 by the paddles 406a, 406b and the gripping members 408a, 408b. In particular, the valve tissue 20,22 is attached to the valve repair device 402 by the optional barbed portion 409 of the gripping members 408a, 408b, and the paddles 406a, 406b engage the gripping members 408 to secure the valve repair device 402 to the valve tissue 20, 22.

In order to move the valve repair device 402 from the open position to the closed position, the lock 407 is moved to an unlocked condition (as shown in FIG. 62) by the lock control mechanism 412. Once the lock 407 is in the unlocked condition, the coupler 405 can be moved along the shaft 403 by the paddle control mechanism 410. In the illustrated example, the paddle control mechanism 410 moves the coupler 405 in a direction Y along the shaft, which causes one paddle 406a to move in a direction X and the other paddle 406b to move in a direction Z. The movement of the paddles 406a, 406b in the direction X and the direction Z, causes the paddles to engage the gripping members 408a, 408b and secure the valve repair device 402 to the valve tissue 20, 22.

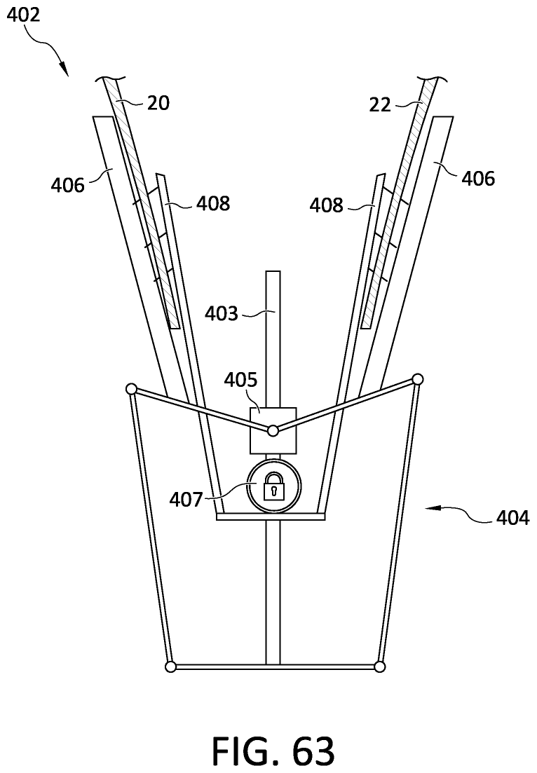

Referring to FIG. 63, after the paddles 406 are moved to the closed position to secure the valve repair device 402 to the valve tissue 20, 22 (as shown in FIG. 62), the lock 407 is moved to the locked condition by the locking control mechanism 412 (FIG. 62) to maintain the valve repair device 402 in the closed position. After the valve repair device 402 is maintained in the locked condition by the lock 407, the valve repair device 402 is removed from the delivery device 401 by disconnecting the shaft 403 from the placement shaft 413 (FIG. 62). In addition, the valve repair device 402 is disengaged from the paddle control mechanism 410 (FIG. 62), the gripper control mechanism 411 (FIG. 62), and the lock control mechanism 412. Removal of the valve repair device 402 from the delivery device 401 allows the valve repair device to remain secured to valve tissue 20, 22 while the delivery device 401 is removed from a patient.

The concepts disclosed by the present application can be used with a wide variety of different valve repair devices. For example, the concepts disclosed by the present application can be used with any of the different valve repair devices disclosed herein. The concepts disclosed by the present application can be used with valve repair devices having paddles, spacers, and other components that can be narrowed and widened, such as the valve repair devices disclosed by U.S. Provisional Application No. 63/278,037, which is incorporated herein by reference in its entirety.

In many of the examples disclosed herein, native valve leaflets are positioned in a gap between components, such as movable and fixed arms of a clasp or between a clasp arm and a paddle, that will be secured to the leaflets. Once the leaflet is positioned within the gap, the component(s) are actuated to pinch the leaflet tissue, thereby securing the leaflet. When the device includes clasps, positioning the leaflet further into the opening between the arms of the clasp before actuating the movable arm to pinch the leaflet allows the movable arm to engage more of the leaflet tissue. Not only is more of the tissue then engaged by the clasp, but any optional barbs or other securing members arranged at the distal ends of the movable or fixed arms are positioned to engage thicker portions of the native leaflet tissue as the tissue is disposed further within the gap. Engaging more and thicker tissue with the clasps ensures a more secure grip on the native leaflet by the clasp.

Determining the depth of native leaflet engagement within the gap between the movable and fixed arms is a challenge using current imaging technology. In particular, the leaflet tissue moves with each beat of the heart and may be translucent or be visually hard to distinguish from surrounding tissue. In contrast, the clasps (e.g., optionally barbed clasps, etc.) formed from materials, such as metal, are easier to see with imaging devices. Therefore, a surgeon can look at the position of the movable arm and one or more indicators to determine whether the clasp has properly engaged the native leaflet.

Example valve repair devices can include an indicator used to determine whether the native leaflet is sufficiently engaged by or within the clasp or optionally barbed clasp during implantation, deployment, or other use of the valve repair device. In some implementations, the indicators are visible via imaging devices during implantation. In some implementations, the indicators generate an electrical signal that indicates leaflet insertion or capture. The indicator can be configured to show or otherwise indicate to the user that the leaflet is inserted in the opening to a desired capture depth and/or that the leaflet has not reached the desired capture depth. Using an indicator allows the user to observe the indicator and/or signals therefrom to determine that the leaflet is properly engaged.

The various indicators herein can be configured in a variety of shapes, sizes, and materials. In some implementations, the indicators can comprise a curved shape, an undulating shape, an S-shape, a C-shape, a U-shape, a V-shape, a hook shape, a check-mark shape, a swoosh shape, a linear shape, a planar shape, a circular shape, a rectangular shape, a triangular shape, etc.

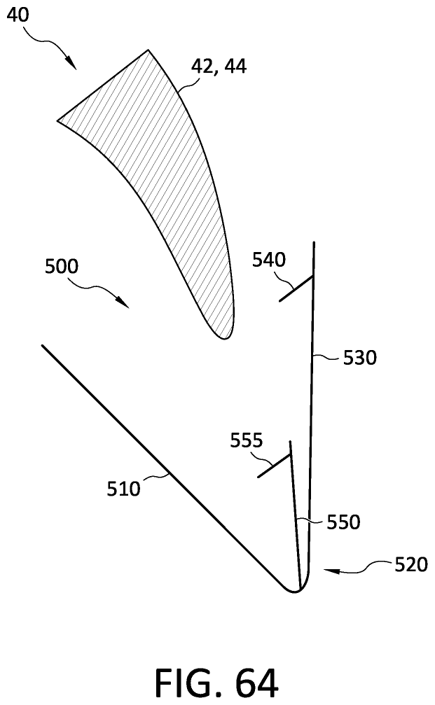

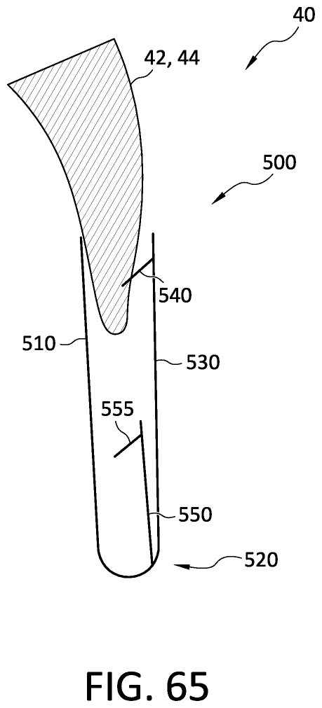

Referring now to FIGS. 64-67, the example clasp 500 (which can be a barbed clasp or include other friction or grip enhancing features) is shown being deployed within a native valve 40, such as the mitral valve, the tricuspid valve, the aortic valve, or the pulmonary valve, to couple a device (not shown), such as any of the devices, valve repair devices, valve treatment devices, implantable devices, implants, etc. described herein, to one of the native leaflets 42, 44. The leaflets 42, 44 can be the mitral valve leaflets 20, 22 or the leaflets of the tricuspid valve, the aortic valve, or the pulmonary valve. Referring now to FIG. 64 the clasp 500 is shown in an open condition with a native leaflet 42, 44 partially inserted into an opening of the clasp 500 formed between the fixed and movable arms 510, 530. To determine whether the leaflet 42, 44 has reached the desired engagement depth, the indicator arm 550 can be actuated via an actuation line (not shown), e.g., an actuation element, actuation suture, actuation wire, etc.). The indicator arm has an optional barb 555 to further secure the leaflet in place. Referring now to FIG. 65, the clasp is shown in a closed configuration, closed on the leaflet 42, 44. The indicator arm 550 has not yet been actuated.

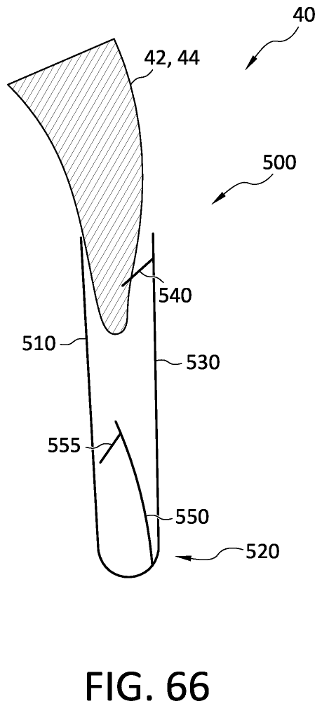

Referring now to FIG. 66, the indicator arm 550 is shown in an actuated condition. The illustrated optional barb 540 on the movable arm 530 has pierced the native leaflet. When the leaflet 42, 44 is inserted into the opening of the clasp 500 halfway or less than halfway between the optional barbed portion 540 and the jointed, flexible, or hinged portion 520 and/or is not inserted far enough into the clasp to overlap with the length of the indicator arm 550, the indicator arm 550 does not engage the leaflet 42, 44. Instead, the indicator arm swings toward the fixed arm 510. The indicator arm's position is visible via imaging devices used to monitor implantation and deployment of the device.

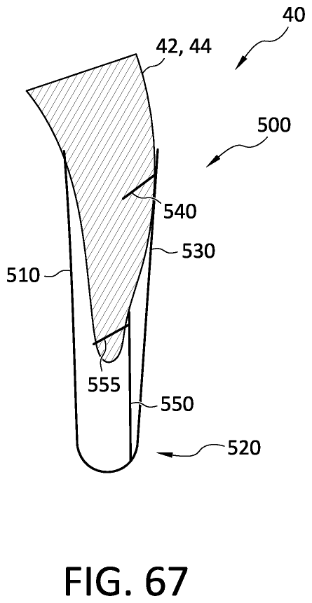

Referring now to FIG. 67, the clasp is closed on the leaflet 42, 44, and the leaflet is positioned deep enough into the clasp 500 such that it overlaps with the indicator arm 550. The optional barb 540 on the movable arm 530 has pierced the native leaflet. The indicator arm rests on the leaflet tissue, and the leaflet prevents or inhibits the indicator arm from moving all the way toward the fixed arm 510 of the clasp. The indicator arm as illustrated in FIG. 67 has an optional barb 540 to further secure the leaflet in place. In examples without a barb on the indicator arm, the indicator arm can bounce with the pulse of the heartbeat, which pulses the leaflet. This pulsing is visible by imaging techniques described above and can be used to indicate to the operator that the leaflet is positioned sufficiently deep into the clasp. Any of the indicators disclosed herein can be configured to pulse or bounce with the leaflets as the heart beats.

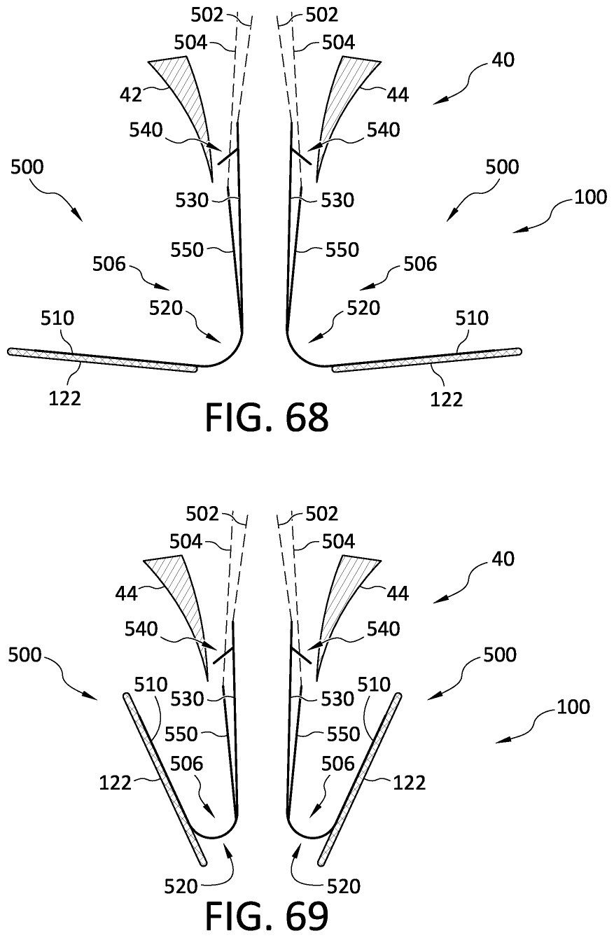

Referring now to FIGS. 68-77, example clasps 500 are shown attached to paddles of a device, such as any of the devices, valve repair devices, valve treatment devices, implantable devices, implants, etc. disclosed herein, and being deployed within a native valve 40 and coupled to one or more of the native leaflets 42, 44. The clasps 500 are attached to paddles 122 of the device 100 that can be moved between opened and closed positions to capture and secure the native leaflets 42, 44 within the device 100, as described above.

Referring now to FIG. 69, the device 100 is shown at the native valve 40 with the paddles 122 opened. The clasps 500 are then opened by applying tension to actuating lines 502, 504 attached to the ends of the movable arms 530 and the indicator arms 550, respectively. The indicator arms disclosed herein can be active (e.g., opened and closed by an active step, such as pulling on the lines 504) or passive (e.g., no addition action in addition to opening and closing of the clasps is needed for operation of the indicator arms). Opening the clasps 500 and the paddles 122 as shown in FIG. 69 allows the device 100 to be maneuvered such that the leaflets 42, 44 are at least partially disposed in the opening 506 formed between the fixed and movable arms 510, 530 of the clasps to facilitate the capture of the leaflets 42, 44 by the clasps 500.

Referring now to FIG. 69, the paddles 122 and clasps 500 are partially closed to position the leaflets for detection by the indicating arms 550 and eventual capture by the clasps 500. The partially closed position of the paddles 122 and clasps 500 allows the optional barbed portions 540 of the movable arms 530 to pinch the leaflets 42, 44 against the fixed arms 510 without stretching or moving the leaflets 42, 44 so far that the leaflets 42, 44 are pushed aside by the movable arms 530 or slip off of the optional barbed portions 540 during an attempted leaflet capture.

Referring now to FIG. 70, both indicator arms 550 are actuated by releasing tension on the actuation lines 504 (e.g., actuation wires, actuation sutures, etc.), which can be the same as or similar to other actuation lines described elsewhere herein. Both indicator arms 550 miss or slip off of the leaflets 42, 44 and move to a fully actuated position that is beyond the fixed arms 510 of the clasps 500. The indicator arms 550 crossing the fixed arms 510 forms an X-shape that is visible via imaging devices used to monitor implantation and deployment of the device.

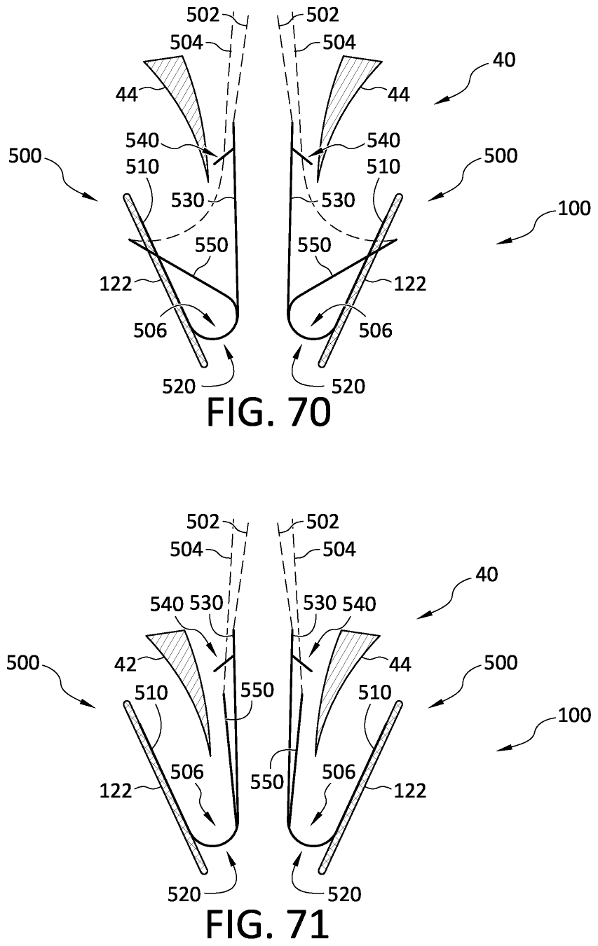

Referring now to FIG. 71, the indicator arms 550 are retracted by applying tension to the actuation lines 504 (e.g., actuation wires, actuation sutures, etc.) and the device 100 is repositioned so that the leaflets 42, 44 are more deeply inserted into the openings 506 of the clasps 500. One of the indicator arms 550 is then allowed to close by releasing tension on one of the actuating lines 504, as can be seen in FIG. 72. The indicator arm 550 engages the leaflet 42 and pinches the leaflet 42 against the fixed arm 510 and paddle 122. FIG. 73 shows the same with the other indicator arm 550 being actuated to engage the other leaflet 44 and pinch the leaflet 44 against the other fixed arm 510 and paddle 122. Engagement with the leaflets 42, 44 prevents or inhibits the indicator arms 550 from moving past the fixed arms 510 of the clasps 500 to form the X-shape shown in FIG. 70. Thus, the indicator arms 550 indicate to an observer observing the installation via an imaging device that the leaflets 42, 44 are inserted into the openings 506 beyond the minimum engagement depth or minimum insertion depth that is determined by the length of the indicator arms 550.

While the terms minimum engagement depth or minimum insertion depth are often used in this disclosure, other similar terms can be used in their place such as an insertion depth, an engagement depth, a selected insertion depth, a selected engagement depth, a preselected insertion depth, a preselected engagement depth, a predetermined insertion depth, a predetermined engagement depth, etc.

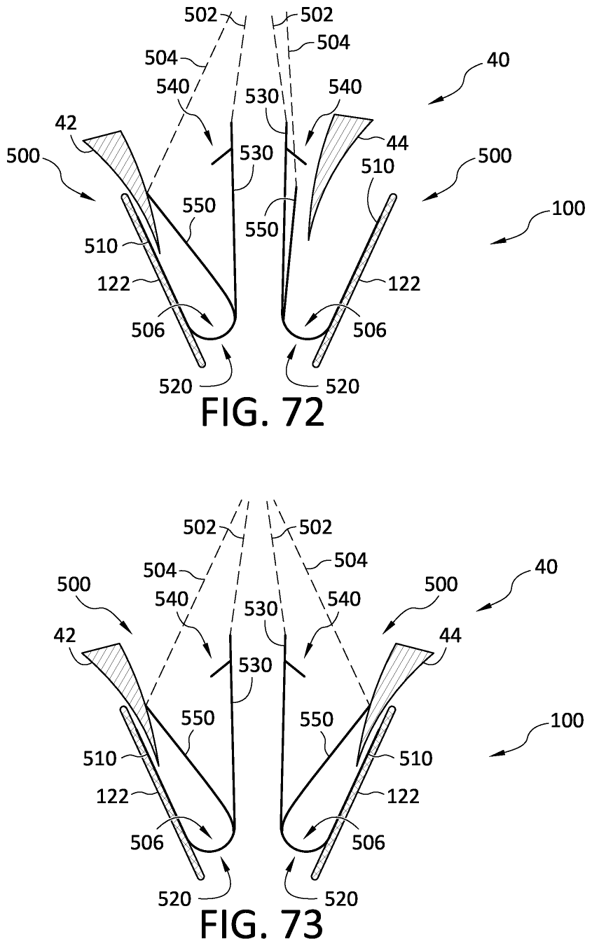

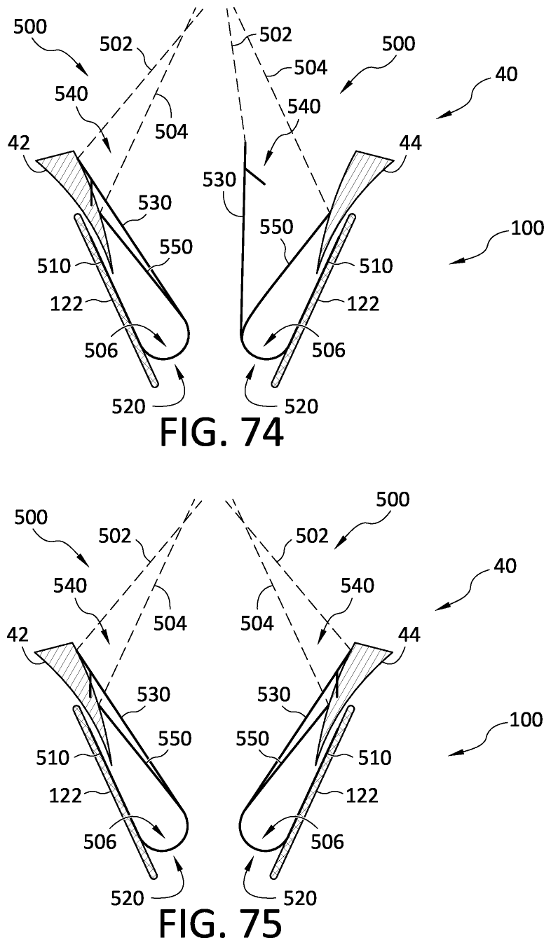

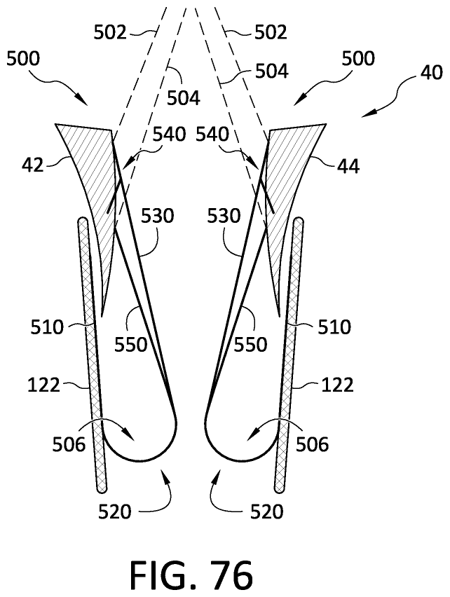

Referring now to FIGS. 74-77, once the indicator arms 550 indicate that the leaflets 42, 44 are sufficiently inserted into the openings 506, the movable arms 530 are actuated by releasing tension on the actuating lines 502 so that the leaflets 42, 44 are pinched between the optional barbed portions 540 and fixed arms 510 of each clasp 500. The paddles 122 are then moved to a fully closed position, shown in FIG. 76, to secure the leaflets firmly within the device 100. The indicators 550 can be monitored in any of the positions illustrated by FIGS. 72-76. For example, the indicators 550 will pulse or jump as the heart beats. This pulsing or jumping can be visualized to confirm that the valve repair device is correctly positioned. Since the indicators 550 are flexible enough to flex or jump as the heart beats, the movable arms 530 can be made stiff and/or close with a high enough force that closed movable arms 530 do not pulse or jump as the heart beats. Any of the indicators disclosed herein can be flexible enough to flex or jump as the heart beats.

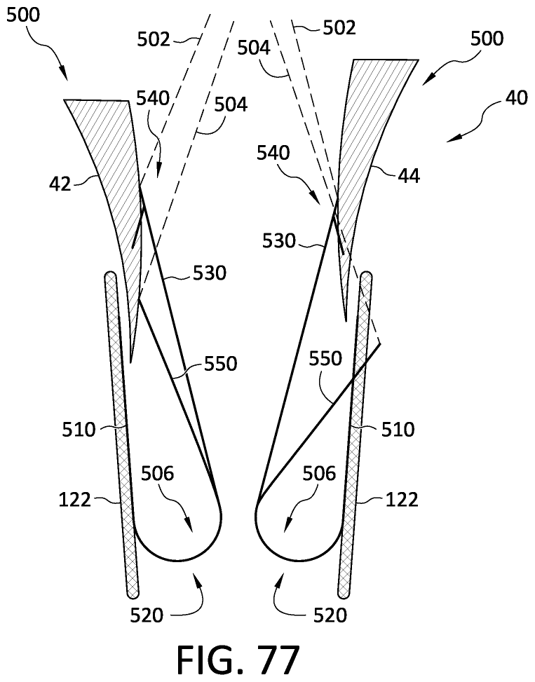

Referring now to FIG. 77, one of the leaflets 44 is shown partially withdrawn from the device 100, which may occur because of movement of the leaflets 42, 44 during the beating of the heart. As can be seen in FIG. 77, the leaflet 44 remains partially secured by the optional barbed portion 540. However, the leaflet 44 is no longer secured at or beyond the minimum engagement depth as determined by the length of the indicator arm 550. Withdrawal of the leaflet 44 allows the indicator arm 550 to move beyond the fixed arm 510, thereby forming an X-shape that is visible to an observer using an imaging device. In addition, or instead, the indicator arm 550 that does not contact the valve leaflet does not pulse or jump as the heart beats. Thus, insufficient retention or slippage of the leaflets 42, 44 from the device 100 can be detected before the device 100 is detached from a delivery device (not shown). Once the slipped leaflet is detected, the clasps 500 and paddles 122 can be opened and repositioned to better secure the slipped leaflet. Any of the indicator arms disclosed herein can be configured to detect a slipped leaflet. In some implementations, a single actuation line can be used to raise and lower the movable arm of a clasp and allow the indicator to move to a leaflet detecting position.

Any of the features of any of the leaflet depth indicators disclosed in PCT patent application publication No. 2020/168,081, which is incorporated herein by reference in its entirety, can be combined with the leaflet depth indicators disclosed herein. The leaflet depth indicators can be used with a variety of devices that grasp leaflets as well. For example, the leaflet depth indicators can be used with the valve repair devices, implants, etc. shown and described in US 2019/0290260, WO 2018167388, as well as chordae repair devices that require grasping the end of the leaflet (see e.g., US 2019/0290260, WO 2018167388).

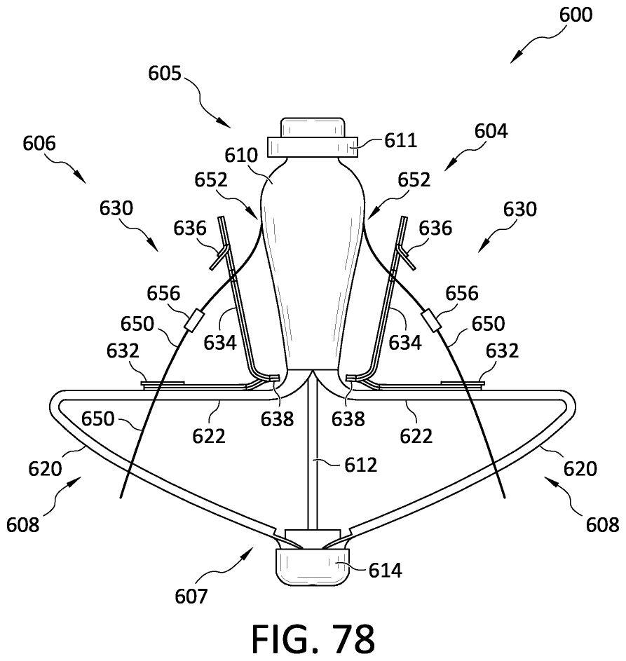

Referring now to FIGS. 78-87, examples devices 600 (e.g., which can be the same as or similar to other devices, valve repair devices, valve treatment devices, implants, etc. described herein) are shown in various positions and configurations ranging from partially open to closed.

As illustrated in FIG. 78, an example device 600 includes a coaptation portion 604, a proximal or attachment portion 605, an anchor portion 606, and a distal portion 607. In some implementations, the coaptation portion 604 of the device optionally includes a coaptation element 610 (e.g., a spacer, coaption element, plug, membrane, sheet, etc.) for implantation between leaflets of a native valve. In some implementations, the anchor portion 606 includes a plurality of anchors 608. The anchors can be configured in a variety of ways. In some implementations, each anchor 608 includes outer paddles 620, inner paddles 622, paddle extension members or paddle frames (not shown), and clasps 630. In some implementations, the clasps 630 include a base or fixed arm 632, a movable arm 634, optional barbs 636, and a joint portion 638. In some implementations, the attachment portion 605 includes a first or proximal collar 611.

Referring to FIG. 78, the device 600 is shown in a laterally extended or open position. The device 600 is moved into the laterally extended or open position by continuing to extend the actuation element 612 described above, thereby increasing the distance between the coaptation element 610 and the cap 614 of the distal portion 607. In the laterally extended or open position, the inner paddles 622 extend horizontally more than in other positions of the device 600 and form an approximately 90-degree angle with the coaptation element 610. Similarly, the paddle frames (not shown) are at their maximum spread position when the device 600 is in the laterally extended or open position. The increased gap between the coaptation element 610 and inner paddle 622 formed in the laterally extended or open position allows clasps 630 to open further before engaging the coaptation element 610, thereby increasing the size of the gap between the fixed and movable arms 632, 634.

To determine whether a leaflet has reached the engagement depth, the device 600 can include an indicator arm 650. The indicator arm 650 can be a variety of shapes and sizes and can be made of a variety of materials. In some implementations, the indicator arm 650 is a wire. The indicator arm 650 can be attached to the device 600 in a variety of locations. In some implementations, a first end 652 of the indicator arm 650 is fixedly attached to the coaptation element 610.

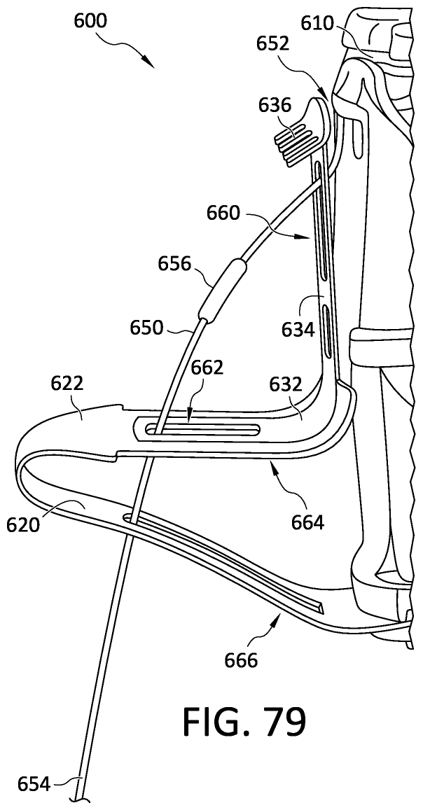

With reference to FIG. 79, in accordance with some implementations, the fixed arm 632, the movable arm 634, the outer paddle 620, the inner paddle 622, and the paddle frames (not shown) can each include one or more channels or slots, through which the indicator arm 650 can be disposed. For example, as shown in FIG. 79, the indicator arm 650 can be disposed through the movable arm channel or slot 660 of the movable arm 634, fixed arm channel or slot 662 of the fixed arm 632, the inner paddle channel or slot 664 of the inner paddle 622, and the outer paddle channel or slot 666 of the outer paddle 620.

The second end 654 of the indicator arm 650 can terminate in various locations. In some implementations, the second end 654 of the indicator arm 650 can terminate distal to the outer paddles 620, while in other implementations, the second end 654 of the indicator arm 650 can terminate between the outer paddle 620 and the inner paddle 622, or between the fixed arm 632 and the movable arm 634. The second end 654 of the indicator arm 650 can also terminate within any of the movable arm channel or slot 660, fixed arm channel or slot 662, inner paddle channel or slot 664, or the outer paddle channel or slot 666. As the device 600 moves and progresses from open to closed, the second end 654 of the indicator arm 650 will move as well. For example, the indicator arm 650 will flatten, align with, and/or be pressed against the device 600 when the device is closed. As a result, the indicator arm 650 does not increase the size or does not significantly increase the size of the device 600.

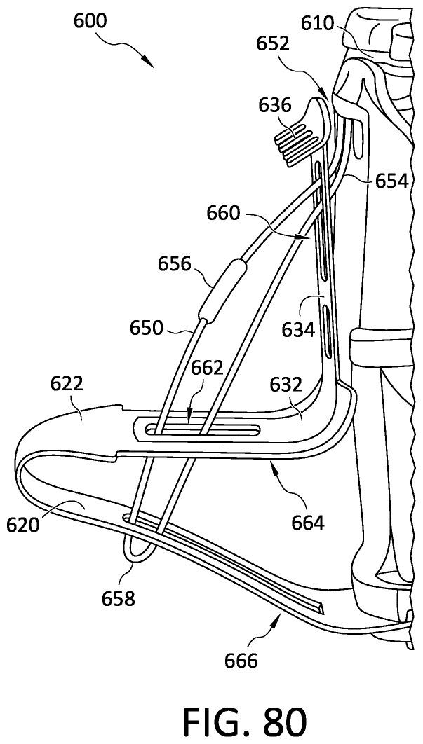

In some implementations, the indicator arm 650 can include any number of loops, turns, bends, or twists between the first end 652 and the second end 654. With reference to FIG. 80, the indicator arm 650 can include a bend 658 between the first end 652 and the second end 654. The bend 658 as shown in FIG. 80 is disposed distal to the outer paddle 620, but the bend can also be disposed between the movable arm 634 and fixed arm 632, or between the fixed arm 632 and the outer paddle 620. Distal to the bend 658, the second end 654 of the indicator arm 650 can be positioned towards, or attached to, the coaptation element 610.

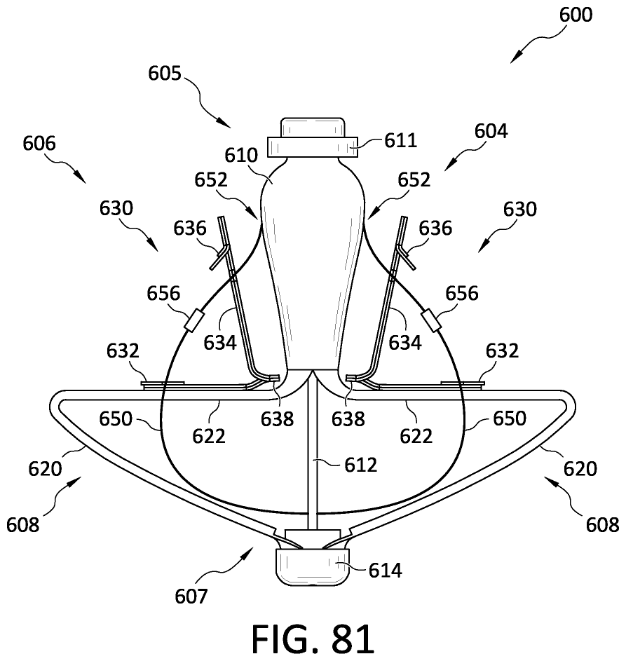

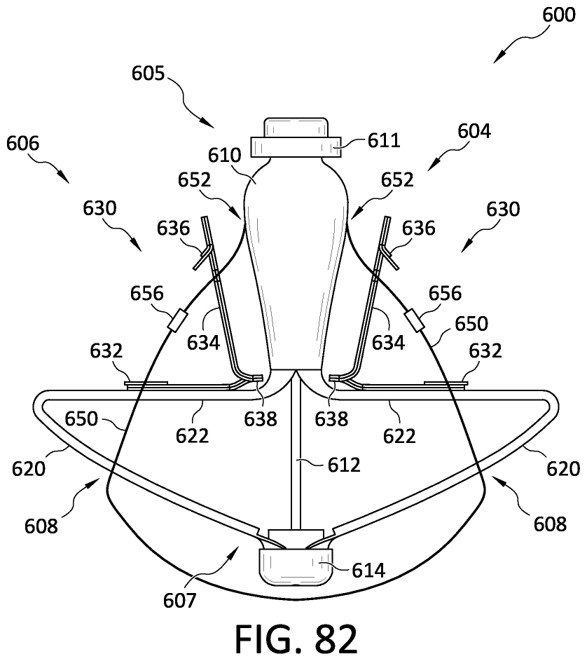

With reference to FIGS. 81 and 82, the indicator arm 650 is attached to the opposing indicator arm 650 of the device 600. For example, the two indicator arms can be formed from a single wire. The single wire can be thin and flexible such that the wire is compressed inside the device 600 when the device is closed. As a result, the indicator arm 650 does not increase the size or does not significantly increase the size of the device 600. In the example illustrated by FIG. 81, the portion that connects the indicator arms is disposed inside the paddles. In the example illustrated by FIG. 82, the portion that connects the indicator arms 650 extends past and/or through the paddles.

The indicator arm 650 can include an indicator marker 656 and/or the indicator arm itself can act as a marker or include portions that act as a marker. The indicator marker 656 can be a radiopaque material that can be printed or attached as a separate piece of material to the indicator marker 656. For example, the radiopaque material can be a coil made of platinum or another radiopaque material. The indicator marker 656 can be visible with fluoroscopy and/or other imaging techniques and can assist the user in determining whether the leaflet is properly positioned in the clasp 630. The indicator marker need not be a separate component. For example, in some implementations, the indicator marker is integral with the indicator arm, e.g., the indicator marker can be portion of the indicator arm that comprises radiopaque material and/or is thicker or has a larger surface area (which can help increase visibility).

The indicator arm 650 can be separately moved relative to the movable arm 634 to facilitate detection of the depth of engagement of the native leaflet between the movable arm 634 and the fixed arm 632 of the clasp 630. In an example, the indicator arm 650 is more resilient and/or flexible than the movable arm 634. This increased resilience and/or flexibility allows the indicator arm to bounce, pulse or jump, while the movable arm 634 provides a firm grip on the leaflet tissue and does not bounce, pulse, or jump. The bounce, pulse or jump of the indicator arm 650 can be viewed using standard imaging equipment to determine that the clasp is correctly engaged with the leaflet tissue.

When viewing with fluoroscopy and/or other imaging techniques, the distance that the indicator arm 650 and the indicator marker 656 move can assist the user in determining whether the leaflet is properly positioned in the clasp 630. If the leaflets 42, 44 positioned within the clasp 630 engage or otherwise actuate the indicator arm 650, then the indicator arm 650 and the indicator marker 656 will move a distance that can be measured using various techniques. The sufficient distance that indicates proper alignment of the leaflets 42, 44 in the clasp 630 can be predetermined by the user. On the other hand, if the leaflets 42, 44 positioned within the clasp 630 do not engage or otherwise actuate the indicator arm 650 the sufficient distance, then adjusting the device 600 can be necessary until the proper alignment is achieved.

The relative positioning of the indicator arm 650 and the indicator marker 656 can assist in determining a minimum engagement depth of the leaflets 42, 44 as measured from the end of the movable arm 634 of the clasp 630 has been achieved. Positioning the indicator marker 656 closer to or further away from the first end 652 of the indicator arm 650 can change the distance that the indicator marker 656 moves when it is engaged by the leaflets 42, 44. For example, when engaged by the leaflets 42, 44, an indicator marker 656 positioned closer to the first end 652 of the indicator arm 650 will not move as great a distance as an indicator marker 656 positioned further from the first end 652 of the indicator arm 650.

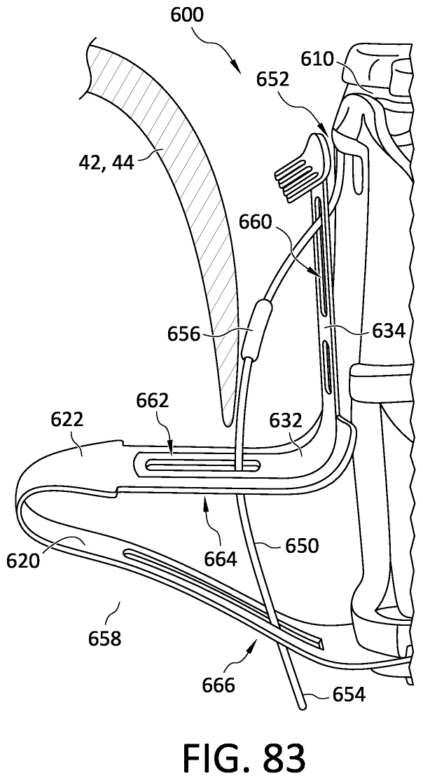

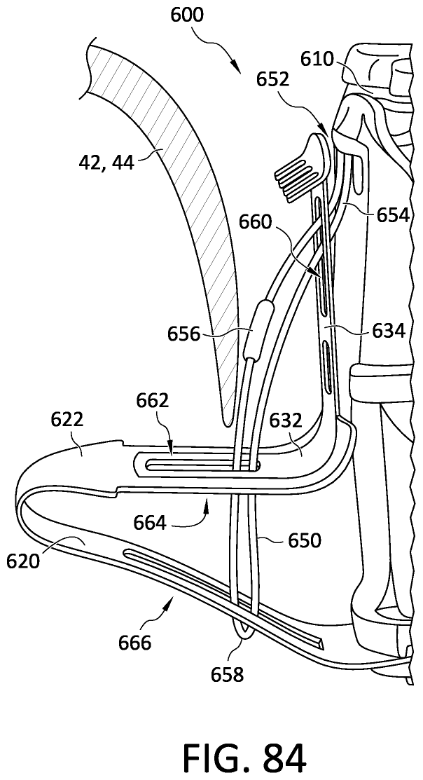

Referring now to FIGS. 83-84, the clasp 630 is shown in an open configuration, with leaflet 42, 44 engaging the indicator arm 650. The leaflets 42, 44 push the marker 656 on the indicator arm closer to the movable arm 634 of the clasp and closer to the coaptation element 610. This movement indicates that the leaflet is positioned at an acceptable depth. Once the user sees that the leaflet is positioned at an acceptable depth, the movable arms 634 and/or the inner and outer paddles 622, 620 are closed to capture the leaflets.

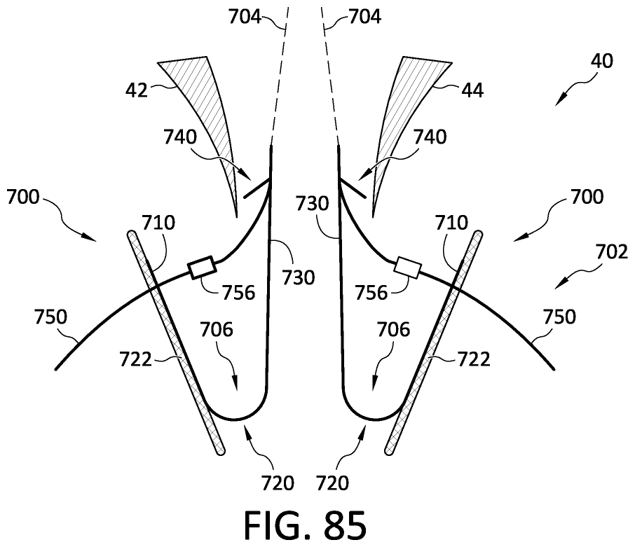

Referring to FIGS. 85-87, example clasps 700 are shown attached to paddles of an example device 702, which is similar in many regards to other devices, valve repair devices, valve treatment devices, implantable devices, implants, etc. disclosed herein, and being deployed within a native valve 40 and to secure the device to the native leaflets 42, 44. Further, the devices illustrated by FIGS. 85-87 are similar to the devices illustrated by FIGS. 75-84, except the indicator arms 750 are connected to the movable arms, instead of the spacer. As such, any of the features of the devices illustrated by FIGS. 85-87 can be used in the devices illustrated by FIGS. 49-64.

Referring now to FIG. 85, the device 702 is shown at the native valve 40 with the paddles 722 opened. The clasps 700 are then opened by applying tension to actuating lines 704 attached to the ends of the movable arm 730. The tension flexes the hinge portion 720 of the clasps to open the clasps. Opening the clasps 700 and the paddles 722 as shown in FIG. 85 allows the device 702 to be maneuvered such that the leaflets 42, 44 are at least partially disposed in the opening 706 formed between the fixed and movable arms 710, 730 of the clasps to facilitate the capture of the leaflets 42, 44 by the clasps 700.

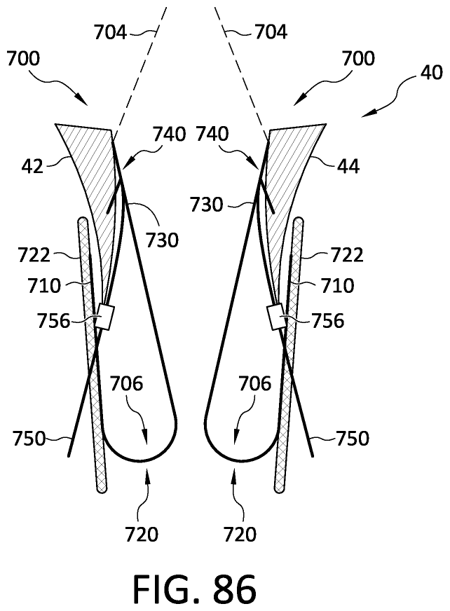

Referring now to FIG. 86, the paddles 722 and clasps 700 are partially closed to position the leaflets for detection by the indicator arms 750 and eventual capture by the clasps 700. The partially closed position of the paddles 722 and clasps 700 allows the optional barbed portions 740 of the movable arms 730 to pinch the leaflets 42, 44 against the fixed arms 710 without stretching or moving the leaflets 42, 44 so far that the leaflets 42, 44 are pushed aside by the movable arms 730 or slip off of the optional barbed portions 740 during an attempted leaflet capture.

Once the indicator arms 750 indicate that the leaflets 42, 44 are sufficiently inserted into the openings 706, the movable arms 730 are actuated by releasing tension on the actuating lines 704 so that the leaflets 42, 44 are pinched between the optional barbed portions 740 and fixed arms 710 of each clasp 700. The paddles 722 are moved to a fully closed position, shown in FIG. 86, to secure the leaflets firmly within the device 702.

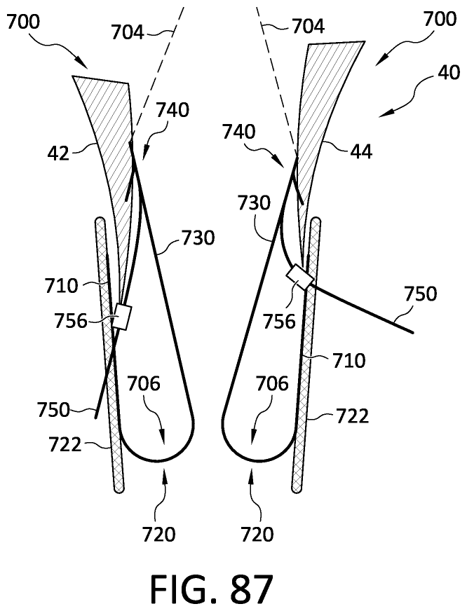

Referring now to FIG. 87, one of the leaflets 44 is shown partially withdrawn from the device 702, which may occur for various reasons, including because of movement of the leaflets 42, 44 during the beating of the heart. The leaflet 44 remains partially secured by the optional barbed portion 740. However, the leaflet 44 is no longer secured at or beyond the minimum engagement depth as determined by the position of the indicator arm 750 and the indicator marker 756. Insufficient retention or slippage of the leaflets 42, 44 from the device 702 can be detected before the device 702 is detached from a delivery device (not shown). Once the slipped leaflet is detected, the clasps 700 and paddles 722 can be opened and repositioned to better secure the slipped leaflet.

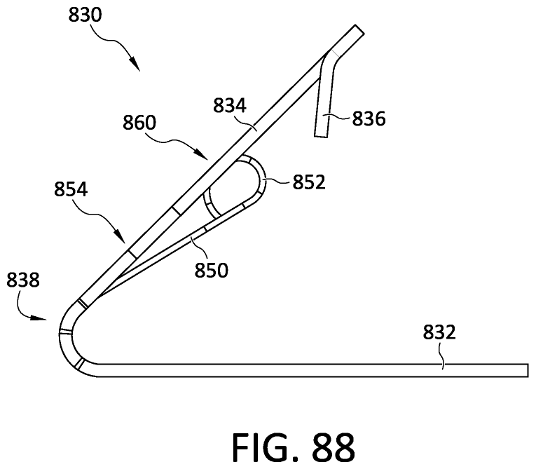

With reference to FIG. 88 a valve repair device clasp 830 includes an indicator arm 850 with a shaped portion 852 that can be used with a valve repair device (see, for example, the valve repair devices disclosed by WO 2020/168081). The clasp 830 includes a fixed arm 832, a flex or hinge portion 838, a movable arm 834 having an optional barbed portion 836, and an indicator 850 connected to the movable arm 834 via an indicator flex or hinge portion 854. The indicator 850 is used to indicate whether the leaflet has reached a minimum depth. The movable arm 834 can have at least one opening 860 in it, through which the indicator passes through. Thus, the shaped portion 852 of the indicator arm 850 will not indicate that the native leaflet has reached a minimum engagement depth until the leaflet is inserted at or beyond the location of the shaped portion 852. Once the leaflet 42, 44 has reached the desired engagement depth, the indicator arm 850 is pressed toward the movable arm 834 by the leaflet 42, 44, causing the shaped portion 852 of the indicator arm to pass through the opening 860 of the movable arm 834. This can be viewed under fluoroscopy because the shaped portion is on the atrial side of the valve. Thus, the shaped portion 852 positioned on the exterior of the movable arm, as opposed to the interior space between the movable and fixed arms 834, 832 of the clasp, indicates that the leaflet 42, 44 has reached a sufficient depth. The shaped portion can be configured in a variety of shapes, e.g., as a circle, square, triangle, rectangle, D-shaped, P-shaped, S-shaped, oval, ovoid, coiled, etc.

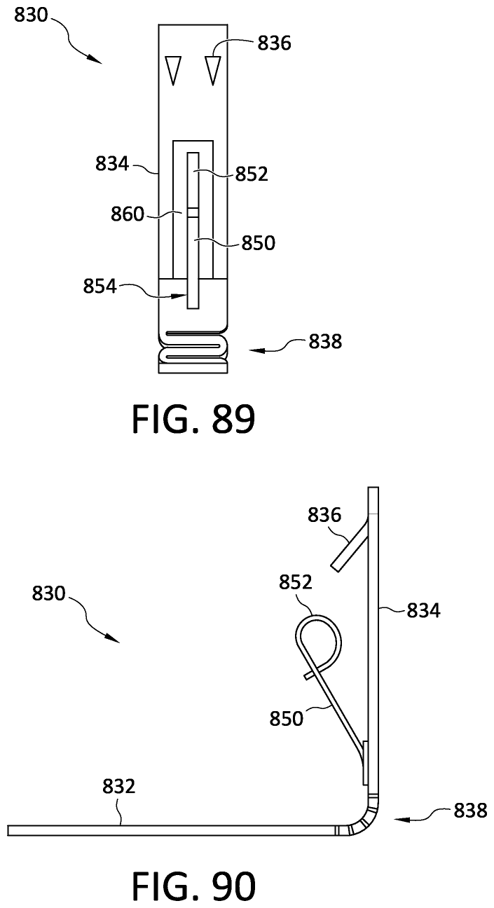

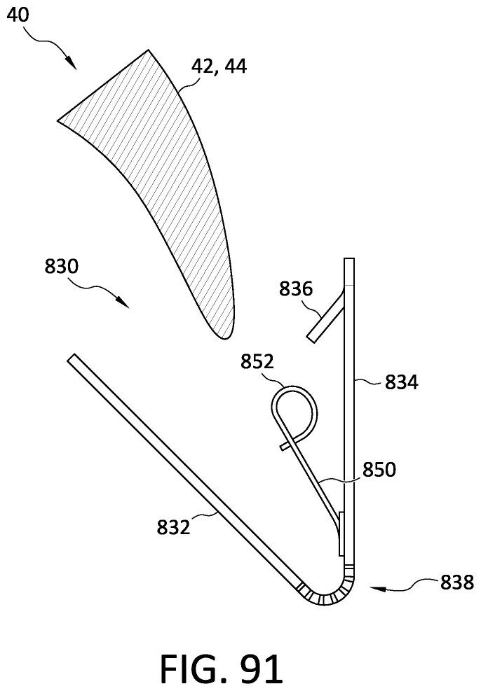

FIGS. 89-90 illustrate the clasp 830 in an open position where the indicator 850 having a shaped portion 852 is in a normal or non-engaged configuration. FIGS. 91-93 illustrate the clasp 830 being deployed within a native valve to secure at least one of the leaflets 42, 44. In FIG. 91, the clasp 830 is shown in an open condition with a native leaflet 42, 44 partially inserted into an opening of the clasp 830 formed between the fixed and movable arms 832, 834. To determine whether the leaflet 42, 44 has reached the desired engagement depth, the movable arm 834 is actuated to close the clasp, such that the movable arm and the fixed arm move closer together. The indicator arm is free to flex, move, or pivot about the indicator flex or hinge portion 854 when pressure is applied to the indicator arm, either by leaflet 42, 44 or the fixed arm 832.

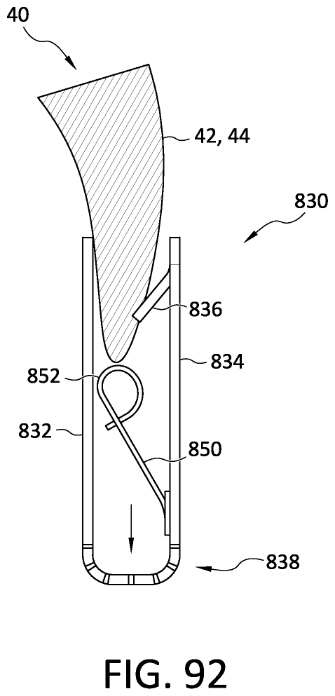

In FIG. 92, when the movable arm 834 is actuated to close the clasp on the leaflet 42, 44, the indicator arm will not be forced out of its resting configuration if the leaflet is not sufficiently deep within the clasp. That is, when the leaflet is not positioned sufficiently deep within the clasp, the indicator arm and shaped portion 852 will remain in its resting configuration between the movable arm and the fixed arm.

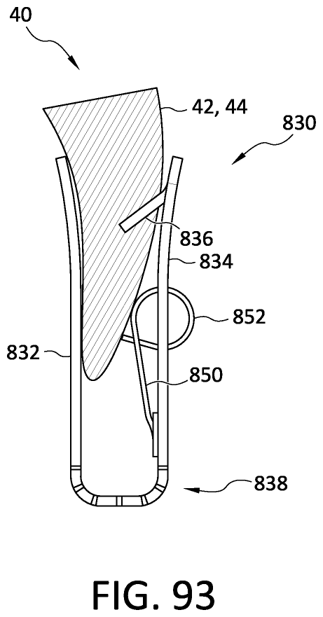

In FIG. 93, the movable arm 834 has been actuated to close the clasp on the leaflet 42, 44, when the leaflet is positioned sufficiently deep within the clasp. The indicator arm 850 and its shaped portion 852 indicate to the operator that the leaflet 42, 44 is sufficiently deep. When the leaflet is sufficiently deep and the movable arm 834 is actuated, the leaflet applies pressure to the indicator arm. This pressure moves the indicator arm 850 towards the movable arm such that the shaped portion 852 of the indicator arm 850 passes through the opening 860 of the movable arm, to the side of the movable arm facing away from the fixed arm (i.e., into open space on the atrial side of the valve leaflets.

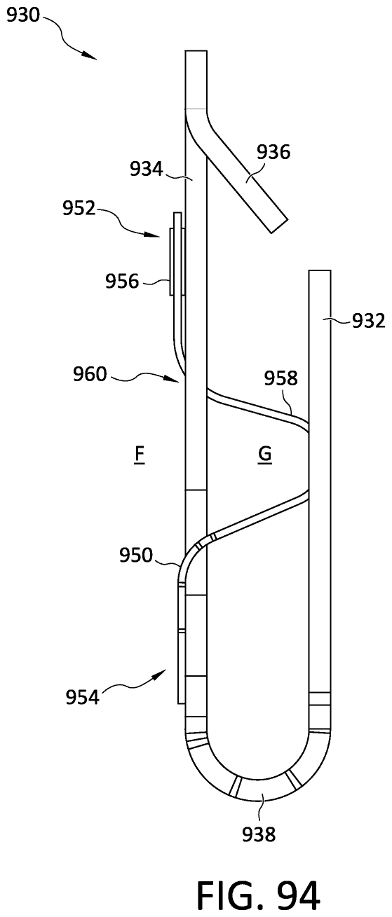

With reference to FIG. 94-98, the clasp 930 includes a fixed arm 932, a flex or hinge portion 938, a movable arm 934 having an optional barbed portion 936, and an indicator arm 950 connected to the movable arm 934. The movable arm 934 can have at least one opening 960 (e.g., aperture, channel, slot, etc.) in it, which the indicator arm 950 passes through. In some implementations, instead of an opening, the indicator arm moves adjacent to the movable arm or through a notch in the side thereof. The indicator arm 950 can include an optional indicator marker 956 and/or the movable clasp arm 934 can include an optional indicator marker 957. Any of the implementations disclosed herein can include the optional indicator marker 956 and/or the optional indicator marker 957. In some implementations, the indicator marker 956 and/or the indicator marker 957 comprise a radiopaque material that can be printed or attached as a separate piece of material to the indicator marker 956 and/or the indicator marker 957. For example, the radiopaque material can be a coil made of platinum or another radiopaque material. The indicator marker is not necessarily a separate component. For example, in some implementations, the indicator marker 956 is integral with the indicator arm, e.g., the indicator marker 956 can be a portion of the indicator arm that comprises radiopaque material and/or is thicker or has a larger surface area (which can help increase visibility) and/or the indicator marker 957 is integral with the movable clasp arm, e.g., the indicator marker 957 can be a portion of the clasp arm that comprises radiopaque material and/or is thicker or has a larger surface area (which can help increase visibility).

The indicator marker 956 and/or the indicator marker 957 can be visible with fluoroscopy and/or other imaging techniques and can assist the user in determining whether the leaflet is properly positioned in the clasp 930. The indicator arm 950 can be used with a suitable valve treatment device or valve repair device (see, for example, the devices disclosed by WO 2020/168081, which is incorporated by reference herein). While some valve repair devices or valve treatment devices may be shown or described herein as implantable devices for illustration purposes, the concepts and configurations described herein (e.g., indicator portions, etc.) can be adapted for use on a variety of devices that are not necessarily implanted and may be removed after treatment.

The indicator arm 950 includes a fixed end 954 and a moving end 952. The fixed end 954 of the indicator arm 950 can be coupled to the movable arm 934 in a variety of ways and at a variety of locations along the movable arm 934. The indicator arm 950 can be coupled to the movable arm 934 at any point between the hinge portion 938 and the optional barbed portion 936.

As shown in FIGS. 94-98, the indicator arm 950 is coupled to the movable arm 934 of the clasp 930 at a first side F of the clasp 930. The indicator arm 950 can pass around or through the movable arm 934 of the clasp 930 such that a portion of the indicator arm 950 is disposed in a second side G of the clasp 930 (opposite the first side F). In some implementations, the indicator arm 950 includes a shaped leaflet-engaging member or leaflet-engaging portion 958 located at least partially on the second side G that can contact a native leaflet when inserted into the clasp 930. Optionally, the leaflet-engaging member or leaflet-engaging portion 958 extends into a space between portions of the fixed arm or a cutout in the fixed arm. This extra extension of the leaflet-engaging member or leaflet-engaging portion 958 facilitates more movement of the marker and/or end portion than would be possible if the leaflet-engaging member or leaflet-engaging portion 958 stopped at the surface of the fixed arm.

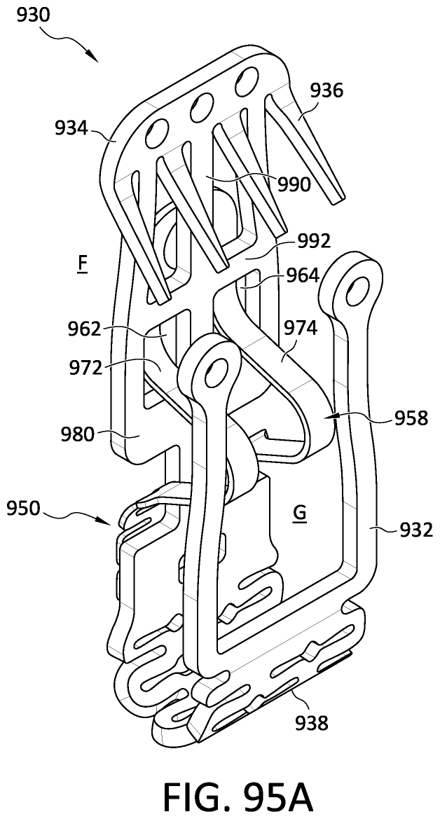

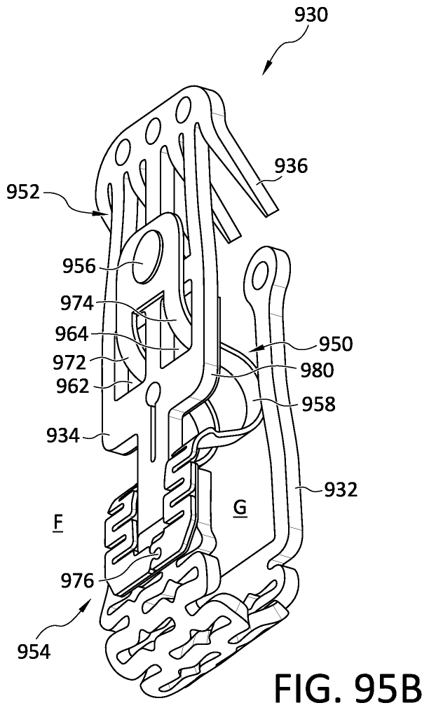

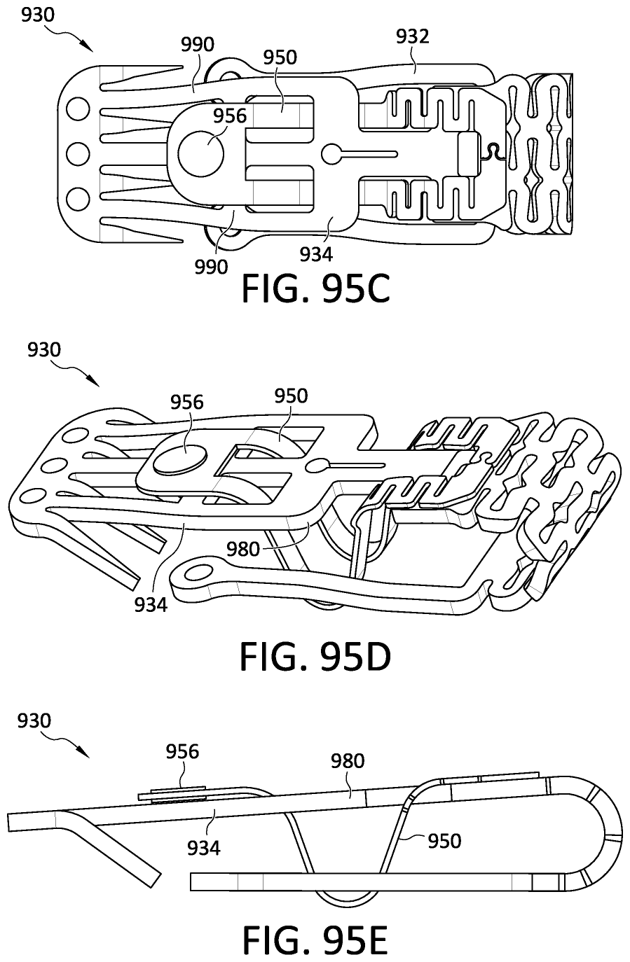

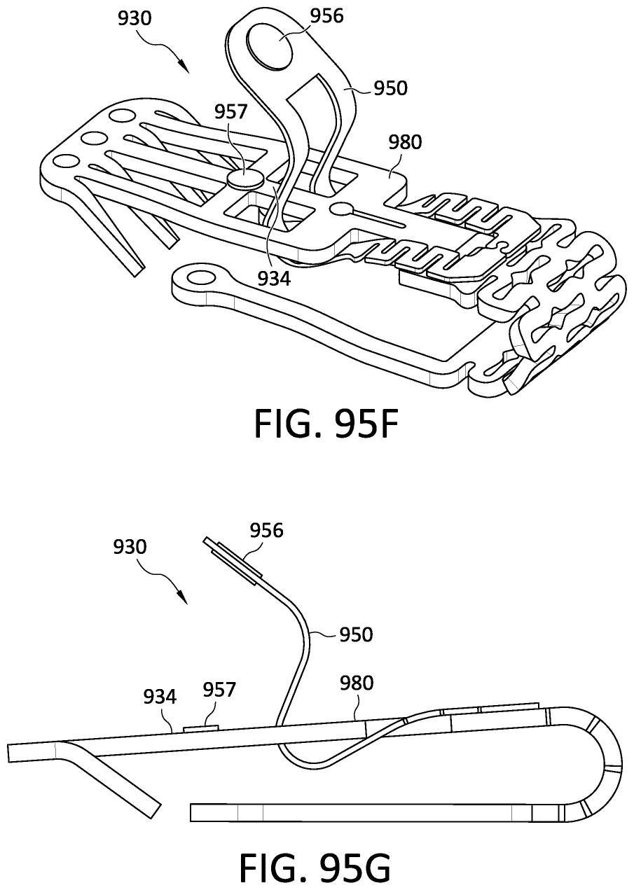

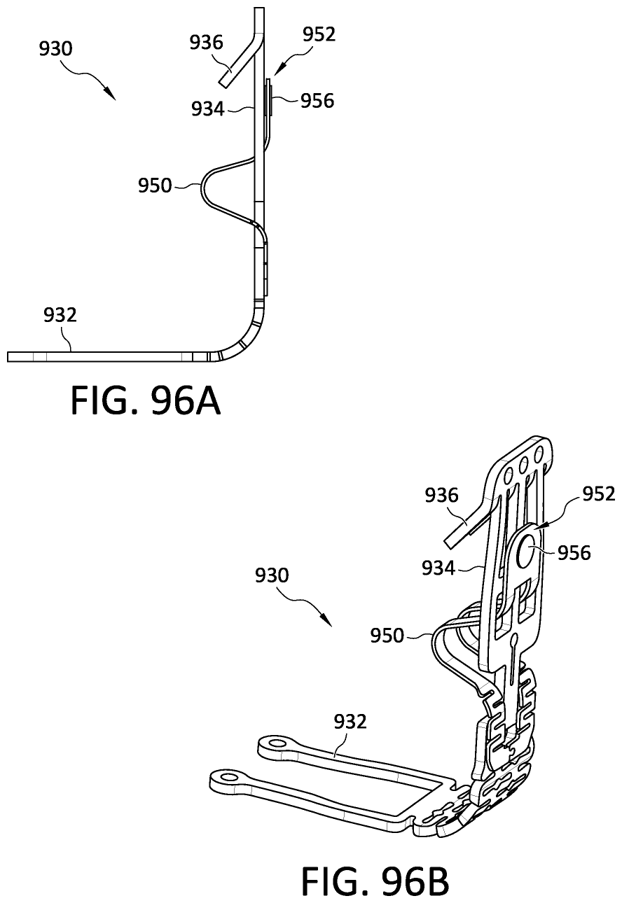

The indicator arm includes one or more arms that extend from the moving end to the fixed end. In some implementations, as illustrated in FIGS. 95A-95G and 96A-96B, the indicator arm 950 can include a first arm 972 and a second arm 974 that extend from the moving end 952 to the fixed end 954 of the indicator arm 950. The first arm 972 and the second arm 974 are connected to the moving end 952 on the first side F of the clasp 930. In some implementations, between the moving end 952 and the leaflet-engaging member or leaflet-engaging portion 958 of the indicator arm 950, the first arm 972 extends through a first opening 962 disposed in the surface of the movable arm 934, and the second arm 974 extends through a second opening 964 disposed in the surface of the movable arm 934 (though other arrangements, e.g., adjacent to or through side notches, etc. are also possible). The movable arm 934 of the clasp includes a first beam 990 and a second beam 992, which extend perpendicular to one another and define the first opening 962 and the second opening 964. The beams 990, 992 define the size of the openings 962, 964 and the path of travel of the indicator arm 950. The beam 990 prevents or inhibits twisting of the indicator. The beam 992 causes the indicator to move substantially orthogonally into the space F when the indicator engages the leaflet tissue.

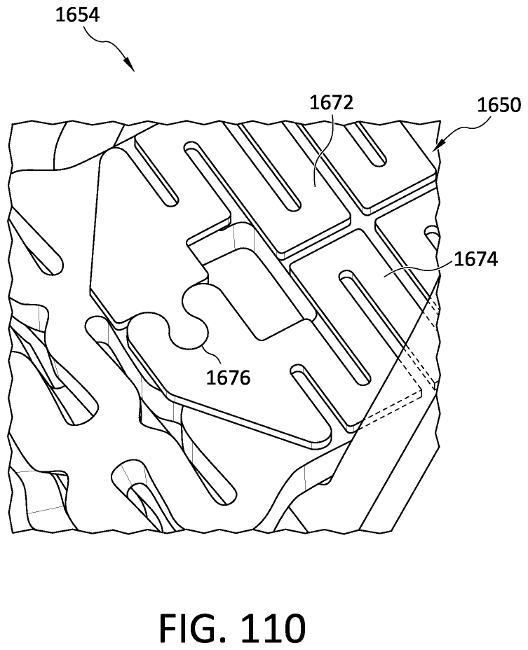

Between the leaflet-engaging member or leaflet-engaging portion 958 and the fixed end of the indicator arm 950, the first arm 972 and the second arm 974 of the indicator arm wrap around the movable arm 934 of the clasp 130 back to the first side F of the clasp 130. At the fixed end 954, the first arm 972 and the second arm 974 can be connected to each other at connection point 976 (see also FIG. 110). At the fixed end 954, the indicator arm 950 is also connected to the movable arm 934 of the clasp 130.

The indicator arm 950 is used to indicate whether the leaflet has reached a desired depth in the clasp 930. Once the leaflet has reached the desired engagement depth in the clasp 930, the leaflet engages the indicator arm 950 on the second side G of the clasp 930. For example, the leaflet can engage one or more of the first arm 972 and the second arm 974 at the leaflet engaging portion 958 of the indicator arm 950. The leaflet-engaging member or leaflet-engaging portion 958 is pressed towards the movable arm 934 of the clasp 930, which causes the moving end 952 and the optional indicator marker 956 (when included) to move away from the movable arm 934 of the clasp 930 and the optional indicator marker 957 (when included). The movement of the indicator marker 956 away from the movable arm 934 of the clasp 930 can be viewed and or measured under fluoroscopy to determine if the leaflet is engaged at the proper position in the clasp 930.

In implementations when both indicator markers 956, 957 are included, an image (e.g., fluoroscopy image) that shows only a single marker (i.e., the two markers 956, 957 are adjacent to one another or abutting one another and only a single mass can be seen on the image) indicates that tissue, such as valve leaflet tissue is not disposed in the clasp 930 to a sufficient depth. Conversely, when both indicator markers 956, 957 are included, an image (e.g., fluoroscopy image) that shows two separate markers (i.e., the two markers 956, 957 are spaced apart) indicates that tissue, such as valve leaflet tissue is disposed in the clasp 930 to a sufficient depth.

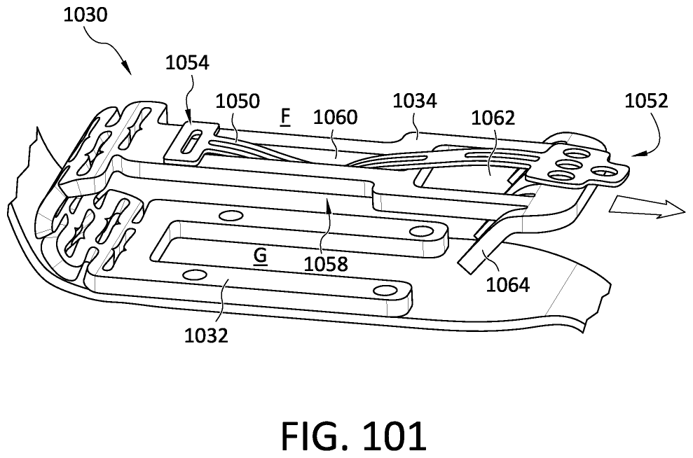

Referring to FIGS. 95A-95G and 96A-96B, the indicator arm 950 can be pushed such that the moving end 952 is moved perpendicular away from the movable arm 934 (FIGS. 95F-95G), or parallel to the movable arm 934 towards the optional barbed portion 936. For example, when the device is partially open and the indicator arm 950 is engaged by a valve leaflet 42, 44 the indicator arm will take the position illustrated by FIGS. 95F-95G to clearly indicate that the leaflet tissue is at a sufficient depth. Referring to FIGS. 96A-96B, when the device is fully closed, the indicator will push against a spacer, central component, and/or actuation element, etc. and be pressed to a flattened condition (see FIG. 101). As a result, a device having the indicator 950 takes up no additional space or very little additional space compared to the same device that does not include the indicator. Optionally, a line or suture can be connected to the indicator 950 to move the indicator to the flattened configuration during the process of capturing the valve leaflets 42, 44. As a result, the indicator would not take up space between the fixed and movable arms 932, 934 during the leaflet capture process.

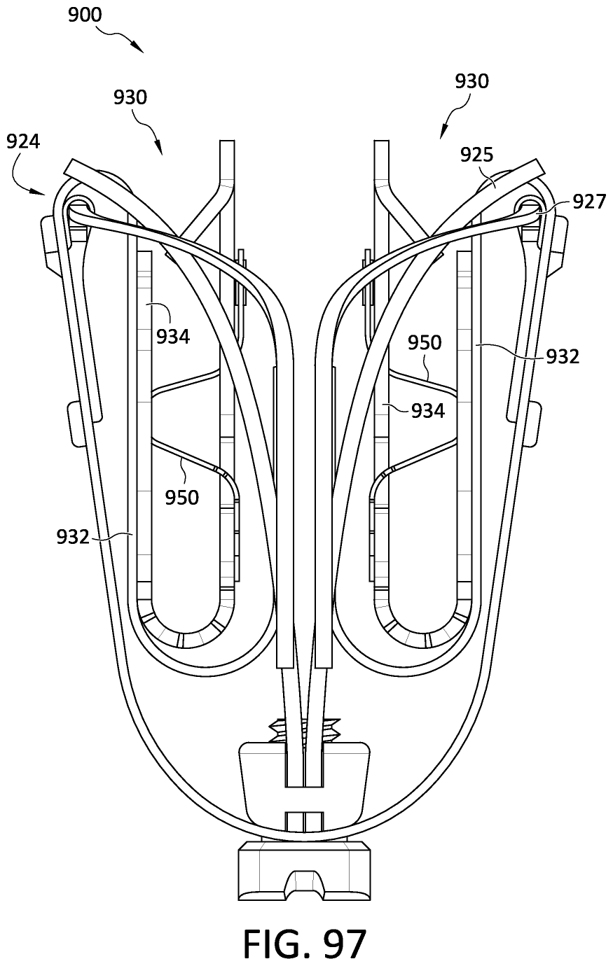

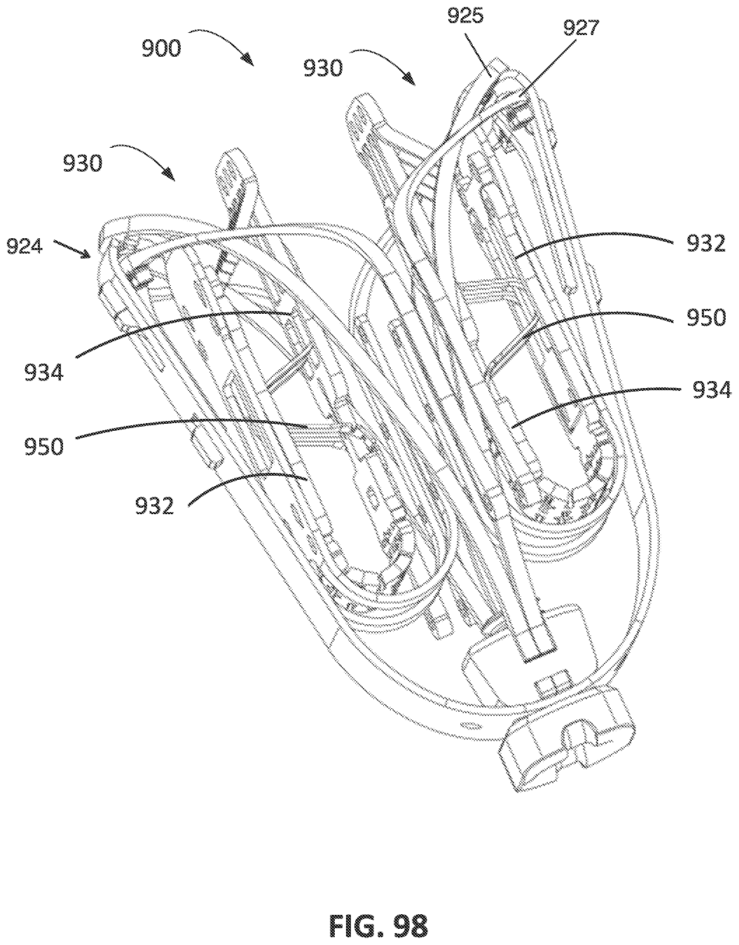

FIGS. 97-98 illustrate a valve repair device 900 that includes the clasps 930 illustrated by FIGS. 95A-95G and 96A-96B. The valve repair device is in the closed configuration in FIGS. 97 and 98. The valve repair device 900 can be operated in substantially the same manner as the valve repair device 200 described above. The valve repair device 900 can optionally include adjustable width paddle frame assemblies 924 instead of the paddle frames 224 of the valve repair device 200. The adjustable width paddle frame assemblies 924 allow the width of the device 900 to be narrowed and widened during deployment of the valve repair device 900. The adjustable width paddle frame assemblies 924 can take a wide variety of different forms. In the illustrated example, the adjustable width paddle frame assemblies 924 include a stiff, inner paddle frame 925, and a flexible, outer paddle frame 927. The stiff, inner paddle frame 925 has a fixed width. The flexible, outer paddle frame 927 has an adjustable width that is controllable and/or settable during deployment of the valve repair device 900.

Referring to FIGS. 95F and 95G, the movable arm 934 can include a cross-bar 980. The indicator arm 950 can contact the cross-bar 980. When the indicator arm 950 is in the engaged position, indicator arm 950 contacts the movable arm 934 of the clasp 930 to limit the movement of the indicator arm 950. As such, the cross-bar 980 acts as a stop for the indicator arm 950 in the engaged position. When the indicator arm 950 is in the disengaged position (FIGS. 95C-95E), the moving end 952 of the indicator arm 950 contacts the movable arm 934 of the clasp 930 to set or limit the position of the indicator arm 950. As such, the back of the movable arm 934 and the moving end 952 act as a stop for the indicator arm 950 in the disengaged position.

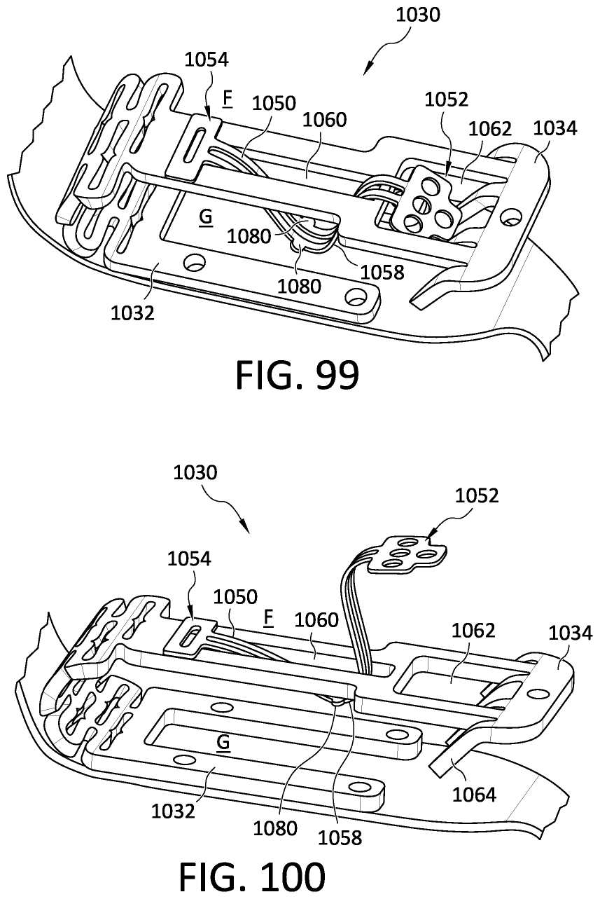

FIGS. 99-101 illustrate an example of a clasp 1030 that operates in a similar fashion to the clasp 930. FIG. 99 illustrates the clasp 1030 in a disengaged position. In some implementations, with reference to FIGS. 99-101, between the fixed end 1054 and the moving end 1052, the indicator arm 1050 extends through a single opening (e.g., opening 1060) of the movable arm 1034 of the clasp 1030. In this example, the fixed arm 1032 has a fork configuration so that in the disengaged position (FIG. 99), the moving end 1052 of the indicator arm 1050 can be disposed parallel to or angled partially into a second opening 1062 in the movable arm 1034. The clasps herein can be used with a variety of valve repair devices or valve treatment devices whether implanted or removed after treatment.

The indicator arm 1050 can include one or more protrusions 1080 that extend outward from the indicator arm 1050. The protrusions 1080 can be located on the leaflet-engaging member or leaflet-engaging portion 1058 of the indicator arm 1050 such that when the indicator arm 1050 is in the engaged position (FIGS. 100-101), the protrusions 1080 engage the movable arm 1034 of the clasp 1030 to prevent or inhibit the leaflet-engaging member or leaflet-engaging portion 1058 from traveling through the opening 1060 and to the first side F. The indicator arm can be pushed such that the moving end 1052 is moved perpendicular away from the movable arm 1034 (FIG. 100), or parallel to the movable arm 1034 towards the optional barbed portion 1064 (FIG. 101). For example, when the device is partially open and the indicator 1050 is engaged by a valve leaflet 42, 44 the indicator arm will take the position illustrated by FIG. 100 to clearly indicate that the leaflet tissue is at a sufficient depth. When the device is fully closed, the indicator will push against the spacer and be pressed to the flattened condition illustrated by FIG. 101. As a result, a device having the indicator 1050 takes up no additional space or very little additional space compared to the same device that does not include the indicator. Optionally, a line or suture can be connected to the indicator 1050 to move the indicator to the configuration illustrated by FIG. 101 during the process of capturing the valve leaflets 42, 44. As a result, the indicator would not take up space between the fixed and movable arms 1032, 1034 during the leaflet capture process.

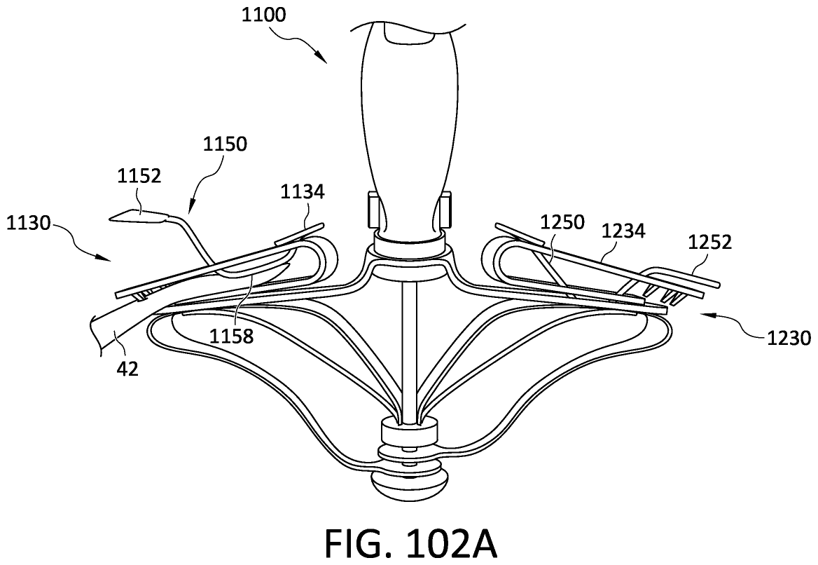

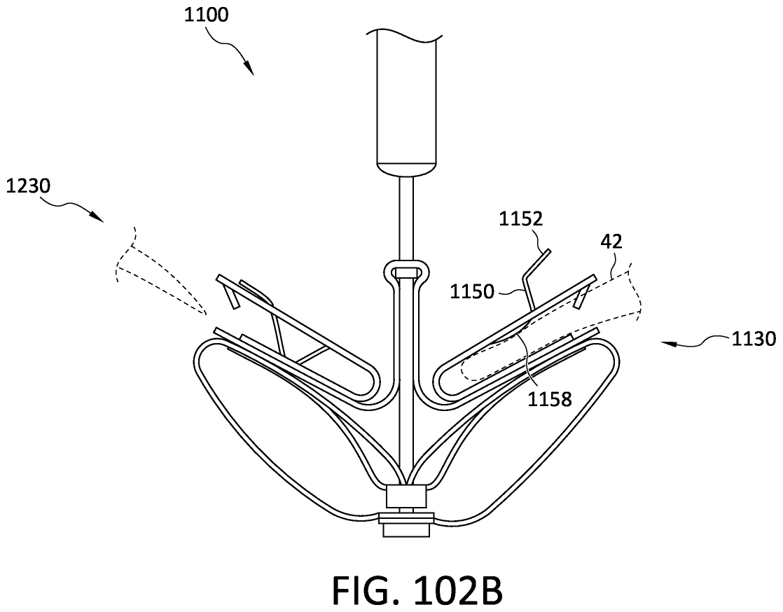

With reference to FIGS. 102A-102B, a device 1100 with two clasps is illustrated. FIG. 102A illustrates the device as viewed in open space. FIG. 102B illustrates the device as viewed under fluoroscopy. A first clasp 1130 is engaged with a leaflet, and a second clasp 1230 is not engaged with a leaflet. The leaflet 42 engages the indicator arm 1150, for example, at the leaflet-engaging member or leaflet-engaging portion 1158, such that the leaflet-engaging member or leaflet-engaging portion 1158 is pressed towards the movable arm 1134 of the clasp 1130, which causes the moving end 1152 and the indicator marker (not shown) to move or extend away from the movable arm 1134 of the clasp 1130. As can be seen in FIGS. 102A and 102B, a clear indication of leaflet capture is provided.

Still referring to FIGS. 102A-102B, the second clasp 1230 is not engaged with a leaflet, and thus the moving end 1252 of the second indicator arm 1250 is adjacent to and/or coupled with the movable arm 1234. The first and second indicator arms 1150, 1250 can be any of the indicators or indicator arms disclosed herein. For example, the indicator arms 1150, 1250 can have any of the features or combinations of the features of the indicator arms shown in FIGS. 94, 95A-95G, 96A, 96B, 99-101, and 103-118.

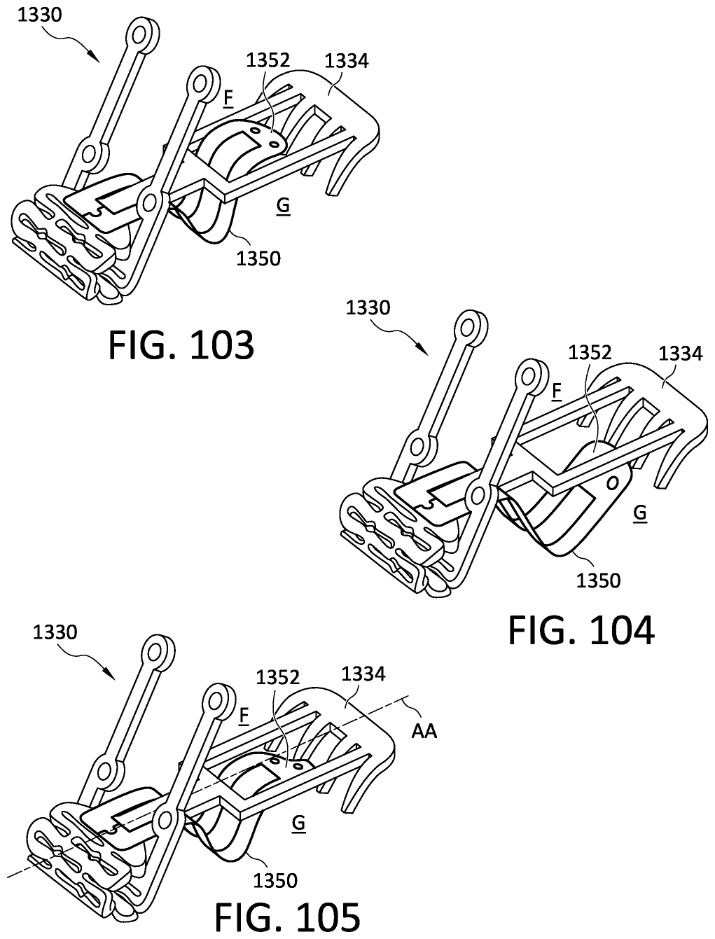

FIGS. 103-105 illustrate additional examples of indicator arm configurations. The indicator arm can be positioned along the clasp in a variety of ways. For example, with reference to FIG. 103, the moving end 1352 of the indicator arm 1350 can be oriented such that in the disengaged position, it is located on the first side F of the movable arm 1334 of the clasp 1330 and bends towards the movable arm 1334. With reference to FIG. 104, the moving end 1352 of the indicator arm 1350 can be oriented such that in the disengaged position, it is located on the second side G of the movable arm 1334 of the clasp 1330 and bends towards the movable arm 1334. With reference to FIG. 105, the moving end 1352 of the indicator arm 1350 can be oriented such that in the disengaged position, it is located within the opening of the movable arm 1334 of the clasp 1330 and extends along the plane AA of the movable arm 1334.

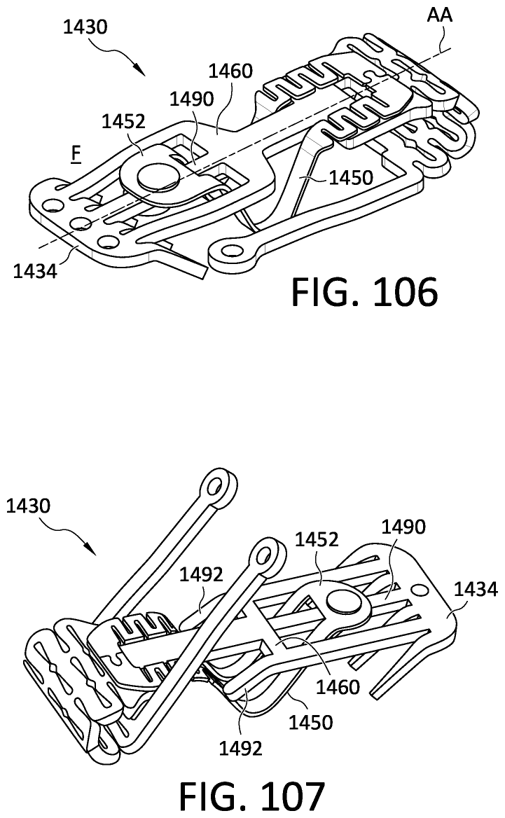

With reference to FIG. 106, the movable arm 1434 of the clasp 1430 can include a bar 1490 extending through the opening along axis AA along the movable arm 1434. The bar 1490 prevents or reduces twisting of the of the indicator arm 1450 by providing a path for legs of the indicator arm 1450 to slide along. In the disengaged position, the moving end 1452 of the indicator arm 1450 is positioned on the first side F of the clasp 1430 adjacent to the bar 1490.

With reference to FIG. 107, the movable arm 1434 of the clasp 1430 can include one or more protrusions 1492 extending from the movable arm 1434. In addition to the bar 1490 or instead of the bar 1490, the protrusions 1492 prevent or reduce twisting of the of the indicator arm 1450 by providing a path for legs of the indicator arm 1450 to slide along.

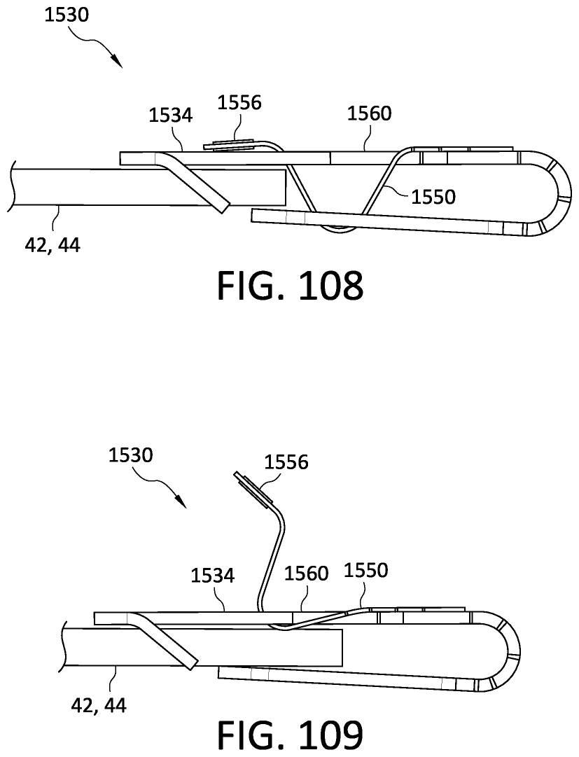

FIGS. 108-109 illustrate the clasp 1530 in a closed position. Referring to FIG. 108, the leaflet is located within the clasp 1530, but is not located far enough within the clasp 1530 to engage the indicator arm 1550. The indicator marker 1556 therefore does not move from the movable arm 1534 of the clasp 1530. The position of the indicator marker 1556, which can be visible with fluoroscopy and/or other imaging techniques, can assist the user in determining that the leaflet 42, 44 is not properly positioned in the clasp 1530. In the FIG. 108 implementation, the indicator marker 1556 rests against a back side of the movable arm 1534 when the leaflet 42, 44 does not engage the indicator arm 1550. As such, the back side of the movable arm 1534 acts as a stop for the indicator arm 1550 in a “not engaged” condition.

Referring to FIG. 109, the leaflet is located far enough within the clasp 1530 to engage the indicator arm 1550. The movement of the indicator arm 1550 results in the movement of the indicator marker 1556 from the movable arm 1534 of the clasp 1530. The position of the indicator marker 1556, which can be visible with fluoroscopy and/or other imaging techniques, can assist the user in determining that the leaflet 42, 44 is properly positioned in the clasp 1530. In the FIG. 109 implementation, the indicator arm 1550 presses against a cross-beam 1560 (see also the similar cross-beams in the implementations illustrated by FIGS. 106 and 107) on the front side of the movable arm 1534 when the leaflet 42, 44 engages the indicator arm 1550. As such, the cross-beam 1560 of the movable arm 1534 acts as a stop for the indicator arm 1550 in an “engaged” condition. As such, the “not engaged” stop of FIG. 108 and the “engaged” stop of FIG. 109 help to provide a clear indication of whether or not the leaflet 42, 44 have been inserted a sufficient depth into the clasp.

With reference to FIG. 110, at the fixed end 1654 of the indicator arm 1650, the first arm 1672 and the second arm 1674 can be connected to each other at connection point 1676. The connection of the first arm 1672 and the second arm 1674 at the connection point 1676 can take place in a variety of ways, for example, by welding, hinge, adhesion, link, interconnection, etc. At the fixed end 1654, the indicator arm 1650 can also be connected to the movable arm of the clasp (see FIG. 96). This split connection point 1676 allows the indicator arms to both be made from a single piece and routed as illustrated by any of FIGS. 95A-95G, 96A, 96B, 97, 98, 103-109. That is, the split connection point 1676 can be spread apart, be routed through openings and/or around bars of the movable arm to position the indicator arm relative to the movable arm, be brought back together, and be secured to the movable arm.

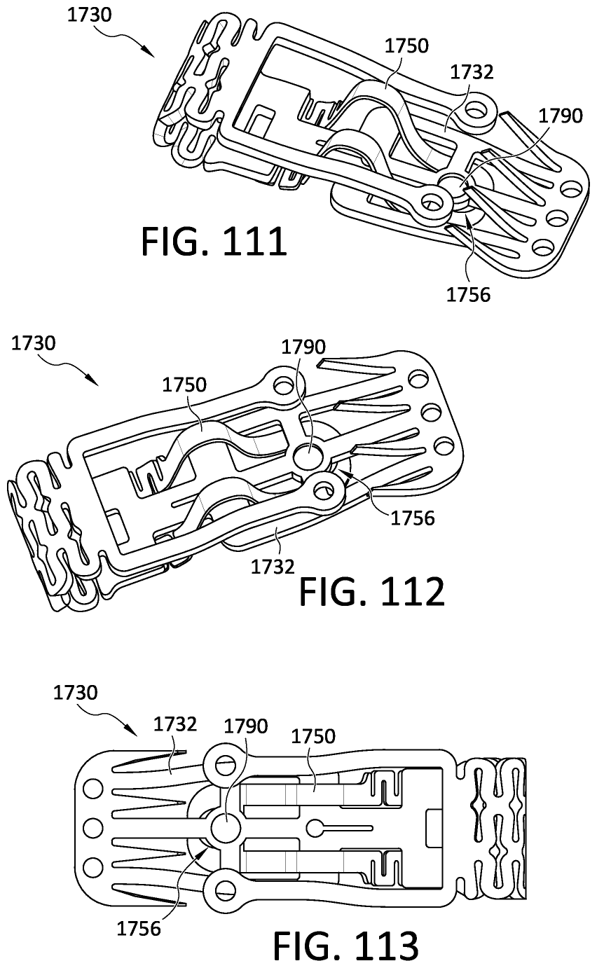

With reference to FIGS. 111-113, the movable arm 1732 of the clasp 1730 can include one or more markers 1790 in addition to a marker on the indicator 1750. The marker 1790 can be a similar substance to an indicator marker 1756 on the indicator arm 1750. The marker 1790 can be a radiopaque material that can be printed or attached as a separate piece of material to the marker 1756. For example, the radiopaque material can be a coil made of platinum or another radiopaque material. The indicator marker need not be a separate component. For example, in some implementations, the indicator marker is integral with the indicator arm, e.g., the indicator marker can be portion of the indicator arm that comprises radiopaque material and/or is thicker or has a larger surface area (which can help increase visibility).

The marker 1756 can be visible with fluoroscopy and/or other imaging techniques and can assist the user in determining whether the leaflet is properly positioned in the clasp 1730. For example, when engaged with a leaflet, the indicator arm 1750 is pushed, resulting in movement of the indicator marker 1756 away from the marker 1790 on the movable arm 1732 of the clasp 1730. The distance between the indicator marker 1756 and the marker 1790 of the movable arm 1732, which can both be visible with fluoroscopy and/or other imaging techniques, can assist the user in determining that the leaflet 42, 44 is properly positioned in the clasp 1730 or is not properly positioned in the clasp 1730.

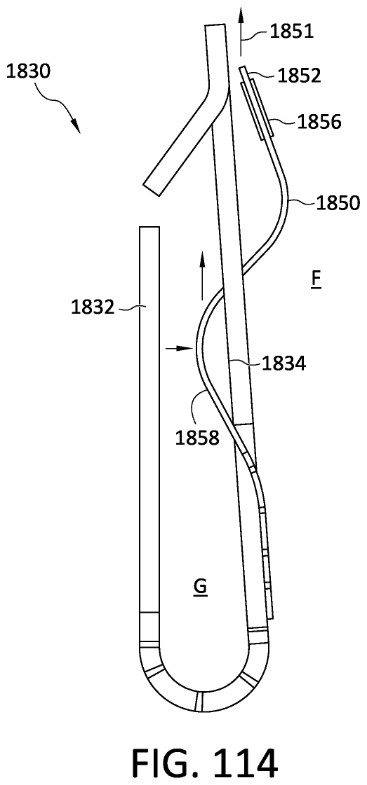

In some implementations the various indicator arms herein can be pulled, stretched, and/or moved to open more capture space. For example, FIG. 114 illustrates that an end 1852 and/or a marker 1856 of an indicator arm 1850 can be pulled on as indicated by arrow 1851 during the process of capturing a valve leaflet 42, 44 between the fixed and movable arms 1832, 1834 of the clasp 1830. For example, the end 1852 of the indicator arm 1850 can be pulled on by a line or suture when the clasp is open. As a result, the indicator arm does not block the space between the fixed and movable arms 1832, 1834 or takes up less of the space between the fixed and movable arms. In the illustrated example, the indicator arm 1850 has a curved path when pulled on as indicated by arrow 1851. For example, the indicator arm can curve, as indicated by reference character 1858 from the attachment between the movable arm and the indicator arm into the space G, but not all the way to the fixed arm. Then the indicator arm 1850 curves back through the movable arm to the space F, but not to the extent that the indicator arm extends when engaged by a valve leaflet. Then the indicator arm 1850 extends back toward the free end of the movable arm 1834. Once the leaflet is positioned in the space G (or the user thinks the leaflet is in the space G), the indicator arm can be released to indicate whether or not the leaflet is disposed in the space G and closing the clasp 1830 will capture the leaflet.

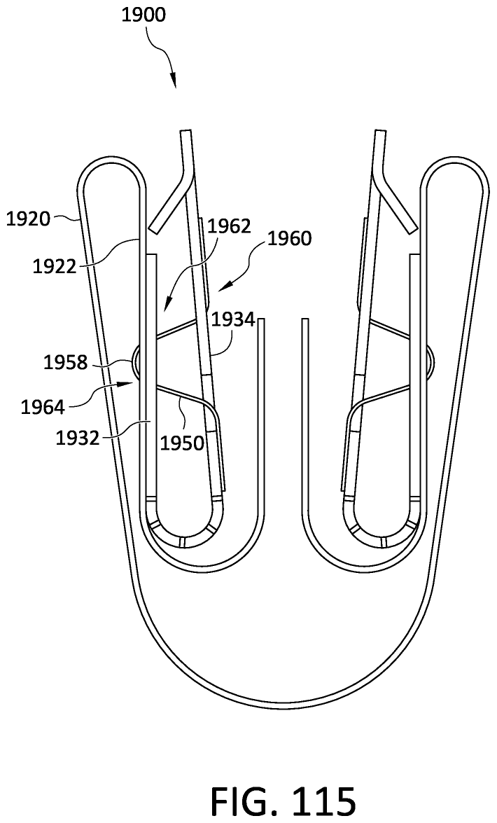

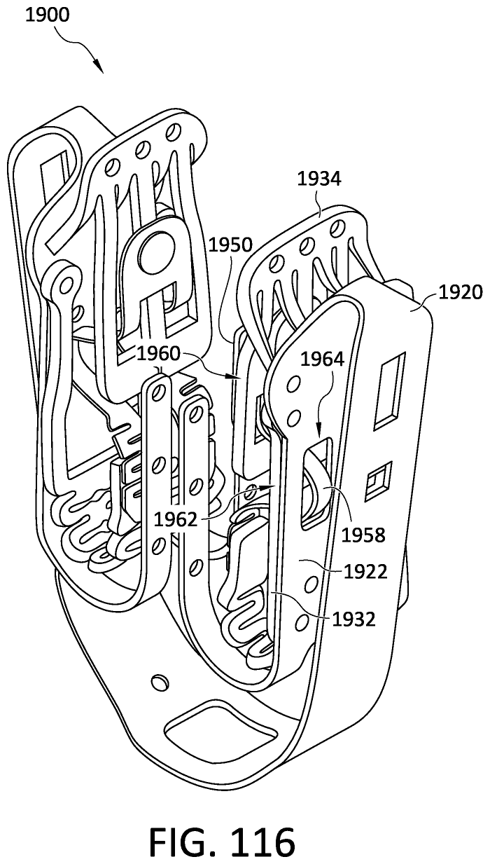

With reference to FIG. 115-116, in accordance with some implementations, one or more of a fixed arm 1932, a movable arm 1934, an outer paddle 1920, the inner paddle 1922, and/or the paddle frames (not shown) of the device 1900 can include openings, channels, cutouts, notches, etc., which the leaflet-engaging member or leaflet-engaging portion 1958 of the indicator arm 1950 can travel through. Otherwise, the fixed arm 1932, the movable arm 1934, the outer paddle 1920, the inner paddle 1922, and the paddle frames (not shown) of the device 1900 can be the same or similar to and/or operate in the same or similar manner as the fixed arm, the movable arm, the outer paddle, the inner paddle, and the paddle frames of the device 200 described above or other devices herein. In some implementations, the fixed arm 1932, the movable arm 1934, the outer paddle 1920, and the inner paddle 1922 can be formed from a single sheet or ribbon of material. In some implementations, as illustrated in FIGS. 115-116, the leaflet-engaging member or leaflet-engaging portion 1958 of the indicator arm 1950 can be disposed through the movable arm channel 1960 of the movable arm 1934, the fixed arm channel 1962 of the fixed arm 1932, and the inner paddle channel 1964 of the inner paddle 1922. By allowing the leaflet-engaging portion 1958 to extend through the fixed arm channel 1962 and the inner paddle channel 1964, the free end of the indicator arm 1950 can extend further from the movable arm 1934 to provide a clearer indication of leaflet engagement.

The devices, clasps, and indicator arms of various devices herein (including, for example, devices 900, 1100, 1900 and clasps 930, 1030, 1130, 1330, 1430, 1530, 1730, 1830, 2030, etc.) can be configured such that, even when the device is in the closed configuration (e.g., as shown in FIGS. 97, 98, 115, and 116), if leaflet tissue is captured within the clasp, then the indicator arm remains extended in an extended position away from the movable arm. Thus, the device can provide an indication of proper capture of the leaflet at the time of capture when the device is in a partially open configuration or capture-ready configuration and, when the device is transitioned from the partially open configuration to the closed configuration, the device can still provide an indication that the leaflet remains properly captured and has not somehow slipped or been torn out of the clasp. This can give the user confidence that the device has been properly implanted, even in the closed configuration.

Further, the devices, clasps, and indicator arms of various devices herein (including, for example, devices 900, 1100, 1900 and clasps 930, 1030, 1130, 1330, 1430, 1530, 1730, 1830, 2030, etc.) can be configured such that the indicator arm can bounce, pulse, or jump in a way that is visible using standard imaging equipment to help determine correct placement and engagement with the leaflet tissue. For example, the devices, clasps, and indicator arms can be configured such that the indicator arm bounces, pulses, or jumps while the leaflet tissue is within the capture region of the clasp before the movable arm of the clasp has been closed. This allows the end user to be sure the leaflet tissue is deep enough to engage the leaflet-engaging portion and will be properly captured before releasing the movable arm to a fully closed position (and thus before penetrating or deeply penetrating the tissue with any optional barbs that can be used on the movable arm of the clasp).

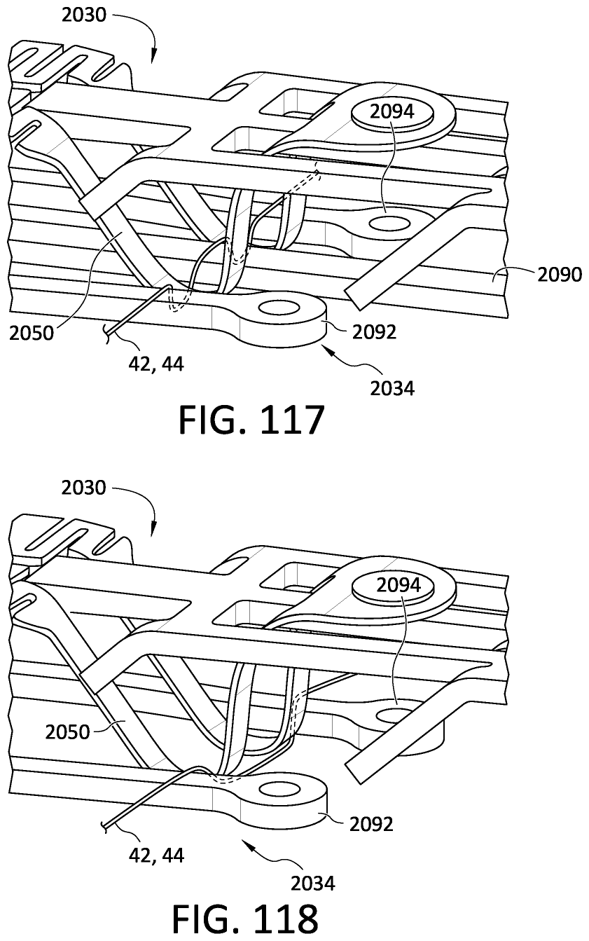

With reference to FIG. 117, the clasp 2030 includes an engaging member 2090 between the first beam 2092 and the second beam 2094 of a fixed arm 2034. The engaging member 2090 can assist in further stabilization of the leaflet 42, 44 when the leaflet 42, 44 is engaged in the clasp 2030 by the indicator arm 2050. Specifically, the leaflet 42, 44 is pressed against the two legs of the indicator arm 2050, the first beam 2092 of the fixed arm 2034, the second beam 2094 of the fixed arm, and the engaging member 2090, leading to further stabilization of the leaflet 42, 44. FIG. 117 illustrates the undulating path of the leaflet 42, 44 when the engaging member is included. FIG. 118 illustrates the clasp 2030 without the engaging member 2090 and the resulting path of the leaflet.

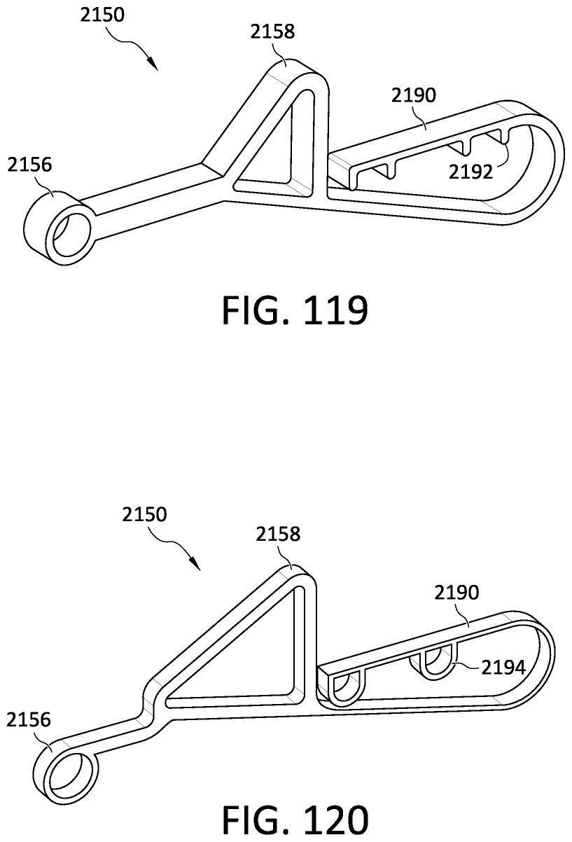

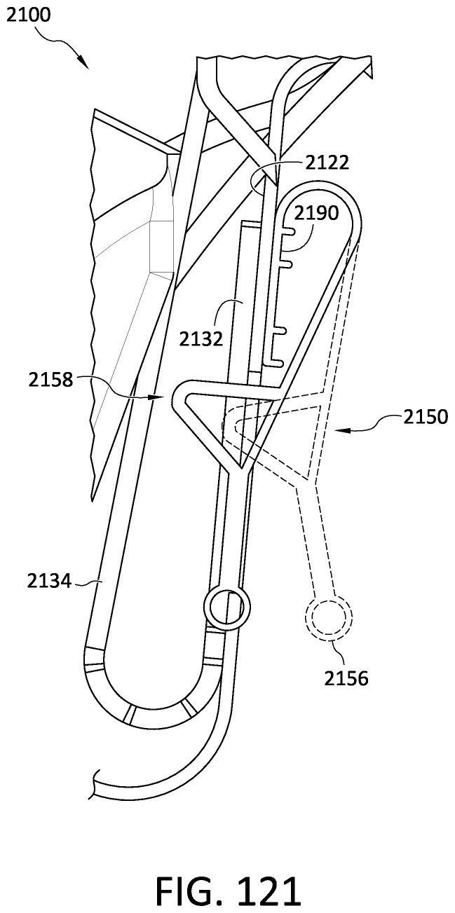

In some implementations, the indicator arm can be coupled with an inner paddle and/or the fixed arm of a clasp of a valve repair device or valve treatment device. The valve repair device can have the configuration of any of the valve repair devices disclosed herein, such as the valve repair device 200. With reference to FIGS. 119-123, the indicator arm 2150 is configured to attach to the inner paddle 2122 (see FIG. 121). This configuration can keep the indicator arm 2150 disposed between the inner paddle and the outer paddle at all times. As such the indicator arms 2150 are contained within the envelope of the valve repair device 2100. The indicator arm 2150 can include a leaflet-engaging member or leaflet-engaging portion 2158 (e.g., an extension, protrusion, arm, edge, bump, dip, swoop, U-shaped portion, V-shaped portion, triangular-shaped portion, curved portion, circular portion, rectangular portion, etc.) for engaging the leaflet and an indicator marker 2156 for assisting the user in determining whether the leaflet is properly positioned in the clasp. The indicator arm 2150 can also include a coupling member 2190 for coupling the indicator arm 2150 to the inner paddle. The coupling member 2190 can be various shapes and sizes and can include pins 2192 (FIG. 119) and/or curves 2194 (FIG. 120) for assistance in coupling. In some implementations, the coupling member 2190 can comprise one or more of a joint, pivot, hinge, pin, clip, clamp, flexible connection, suture, ribbon, bridge, sheet, etc. The indicators (e.g., indicator arms, markers, sensors, electrodes, etc.) herein can be used with a variety of valve repair devices or valve treatment devices whether implanted or removed after treatment.

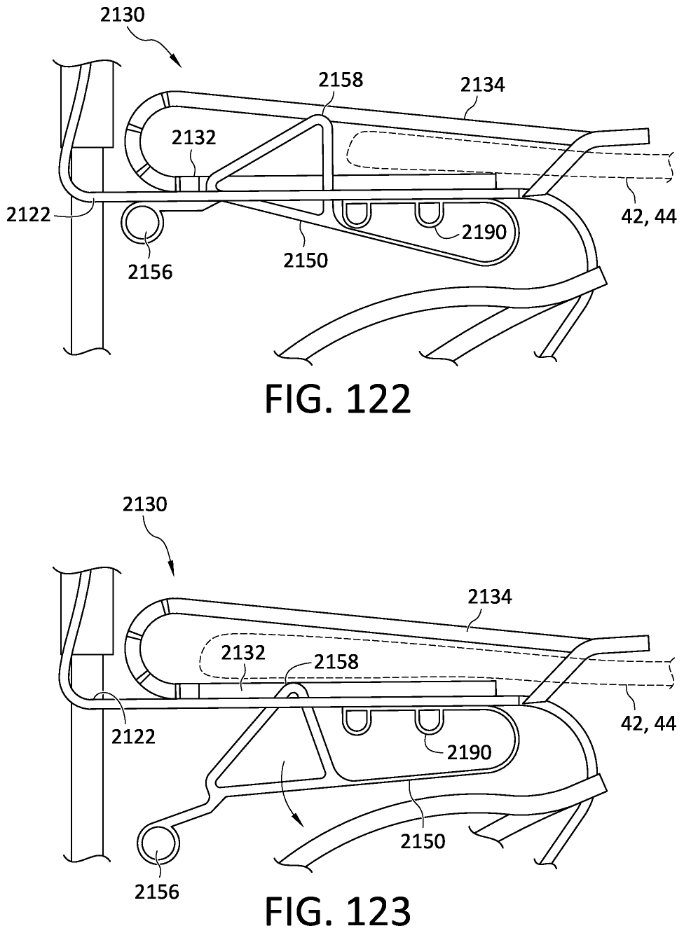

With reference to FIGS. 121-123, the device 2100 with a clasp 2130 including a movable arm 2134, a fixed arm 2132, and an indicator arm 2150 coupled to the inner paddle 2122 via the coupling member 2190. When the leaflet 42, 44 is not located far enough within the clasp 2130 to engage the indicator arm 2150 (see FIG. 122), the indicator marker 2156 lays against the inner paddle 2122. When the leaflet 42, 44 is located far enough within the clasp 2130 to engage the indicator arm 2150, the indicator marker 2156 moves away from the inner paddle 2122 (FIG. 123). The position of the indicator marker 2156, which can be visible with fluoroscopy and/or other imaging techniques, can assist the user in determining that the leaflet 42, 44 is properly positioned in the clasp 2130.

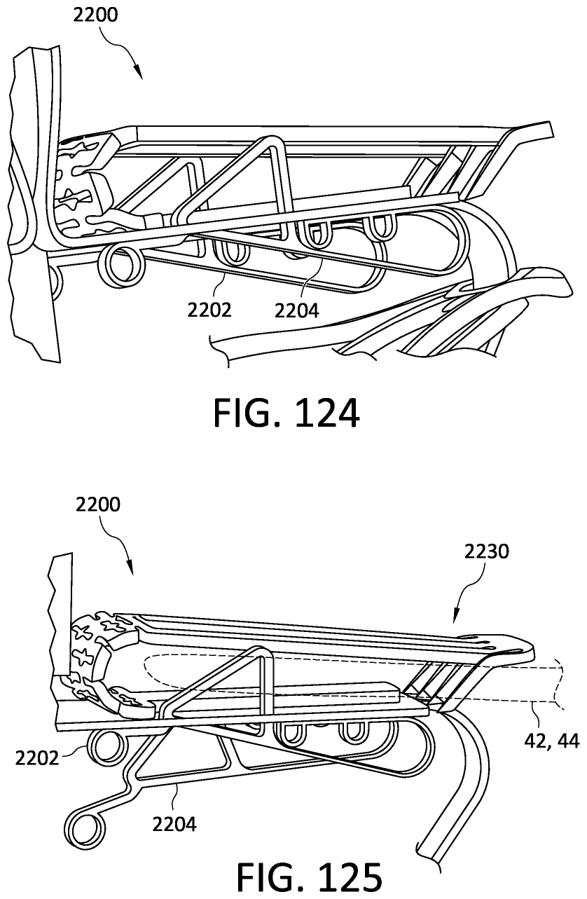

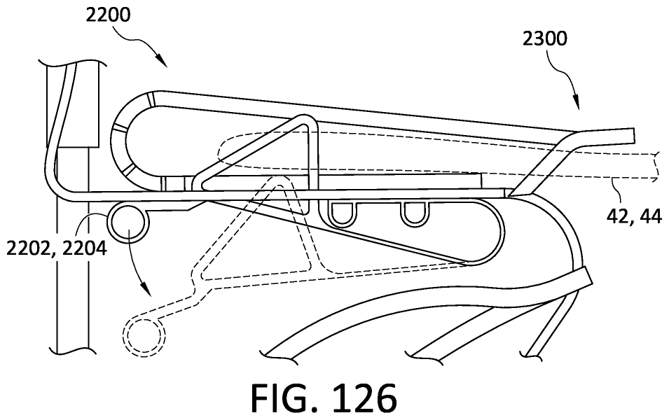

In some implementations, the device can include multiple indicators coupled to the clasp. Any of the clasps disclosed herein can include two or more indicators. For example, with reference to FIGS. 124-126, the device 2200 includes a first indicator arm 2202 and a second indicator arm 2204. The first indicator arm 2202 and the second indicator arm 2204 can be substantially similar to the indicator arm 2150 of FIGS. 121-123. However, any of the indicator configurations disclosed herein can be used and/or indicators disclosed herein can be broken in half or have portions that are broken in half two provide two indicating portions. Having multiple indicators next to each other (e.g., with leaflet-engaging portions as similar depths or distances from the clasp hinge and/or optional clasp barbs/friction-enhancing features) allow a user to determine if the leaflet is properly oriented (e.g., not significantly angled) in the clasp, or whether the leaflet is positioned too far off of one side or another of the clasp. For example, with reference to FIG. 125, the leaflet 42, 44 is positioned withing the clasp 2230 such that it engages the second indicator arm 2204, but not the first indicator arm 2202. This can be determined by locating the indicator marker on fluoroscopy and/or other imaging techniques. This can be due to the device being tilted with respect to the valve leaflets. With reference to FIG. 126, the device 2200 can be readjusted so that the leaflet engages both the first indicator arm 2202 and the second indicator arm 2204, signifying a secure fit of the leaflet 42, 44 within the device 2200 with an acceptable orientation of the clasp on the leaflet. In some implementations, the multiple indicators can have leaflet-engaging portions can be at different depths (or different distances from the clasp hinge or optional clasp barbs/friction-enhancing features) such that you can tell if the leaflet is partially engaged in depth or fully engaged.

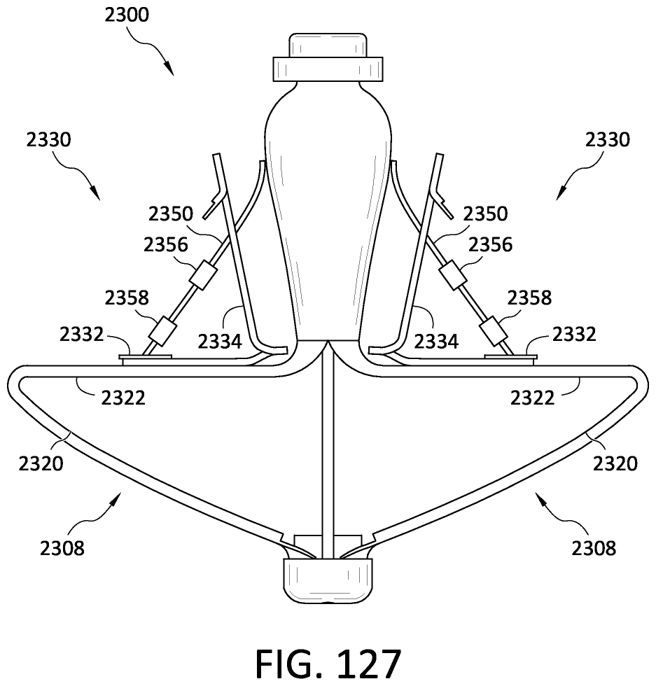

With reference to FIGS. 127-135, leaflet depth can be determined by analyzing electrical signals from electrodes placed on a valve repair device. The electrodes can be placed at a wide variety of different locations of the valve repair device. For example, electrodes can be placed on a visual indicator, such as any of the indicators disclosed herein, a portion of the device, such as on a clasp, on a paddle, on a spacer, etc. When electrodes (or other electrical measuring components) are placed on a visual indicator, the leaflet depth can both be determined through imaging and by analyzing the electrical signals.

The measured signals can take a wide variety of different forms. For example, the signals can include intracardiac electrocardiogram (IECG) signals and bioimpedance signals. The signals measure the electrical activity of the heart during contraction. It has been surprisingly discovered that when electrical signals are measured during leaflet capture, the amplitude and shape of the electrical signals are distinct in instances where the electrodes make contact with the leaflet or other portion of the heart valve (e.g., chordae tendinea). The electrical signals can differentiate the type of tissue that is being contacted, and the extent of that contact with the electrode (i.e., if the electrode is at the edge or near the root of the leaflet). As such, by placing electrodes on the device, the electrical signals can assist the user in determining if the leaflet is captured or partially captured in the device, whether no tissue is captured by the device and/or whether the device is making contact with chordae tendinea or other portion of the heart valve instead of the leaflet.

In the example illustrated by FIG. 127, an example implantable valve repair device or valve treatment device includes a plurality of anchors 2308. The anchors can be configured in a variety of ways. In some implementations, each anchor 2308 includes outer paddles 2320, inner paddles 2322, paddle extension members or paddle frames (not shown), and clasps 2330 having fixed and movable arms 2332, 2334. The device can take a wide variety of different forms. In some implementations, the device 2300 is the same or similar to the device 200 described herein. While the example shown in FIG. 127 is an implantable device, similar configurations and concepts described relative to FIG. 127 can be used on other devices, e.g., valve repair devices, etc., that are not necessarily implanted and may be removed after treatment.

In some implementations, to determine whether a leaflet has reached a particular engagement depth, the device 2300 can include an indicator arm 2350. The indicator arm can include one or more electrodes that measure electrical signals to assist the user in determining if the leaflet is captured or partially captured in the device. For example, the indicator arm 2350 can include a first electrode 2356 and a second electrode 2358. Each of the first electrode 2356 and the second electrode 2358 provide a signal in and/or in contact with material inside the heart at two different locations. For example, the electrodes can provide a signal based on being positioned in blood in the atrium (and not in contact with tissue), based on being positioned in blood in the ventricle (and not in contact with tissue), based on being in contact with valve leaflet tissue and/or based on being in contact with chordae tissue. In some implementations, three, four, five, or more electrodes are included. Any number of electrodes can be included for each clasp.

The electrical signals can take a wide variety of different forms and can be processed in a wide variety of different ways to determine the position of the device in the heart and/or the position of the leaflets relative to the heart. In some implementations, IECG signals are measured on the first and second electrodes 2356, 2358. A bipolar signal can be calculated as the signal from the first electrode 2356 subtracted from the signal from the second electrode 2358. The resulting bipolar signal and/or the original signals can provide indications of the first and/or second electrodes 2356, 2358 being in the atrium (and not in contact with tissue), being positioned in blood in the ventricle (and not in contact with tissue), being in contact with valve leaflet tissue and/or being in contact with chordae tissue.

When measuring the bioimpedance signals, different signal readings will be seen for leaflets which make contact with one electrode or with both electrodes. For example, if a leaflet makes contact with only the first electrode, then a higher magnitude signal reading can result. However, when the leaflet makes sufficient contact with both the first electrode 2356 and a second electrode 2358, then a lower magnitude signal reading can result, indicating that the device is correctly placed.

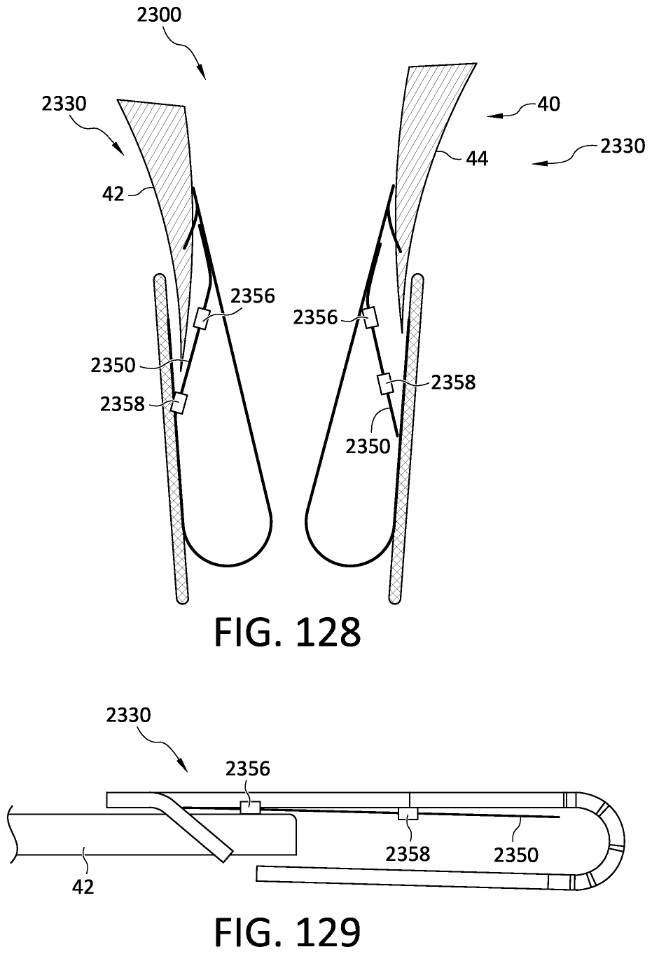

With reference to FIGS. 128-129, the clasps 2330 of the device 2300 can be partially closed (FIG. 128) or fully closed (FIG. 129) such that the position of the leaflets 42, 44 can be detected by the indicating arms 2350 for eventual capture by the clasps 2330. Leaflet 42 is partially secured within the clasp 2330 and makes contact with only the first electrode 2356. Leaflet 44 is partially secured within the clasp 2330 but does not make contact with either the first electrode 2356 or the second electrode 2358. FIG. 129 illustrates the partial capture of leaflet 42 within the clasp 2330. The electrical signals from the first electrode 2356 and the second electrode 2358 can indicate to a user that the leaflets 42, 44 are in an insufficient position and that repositioning of the clasps 2330 is required. The device 2300 can be detached and reattached so that the leaflets can be recaptured within the clasps 2330.

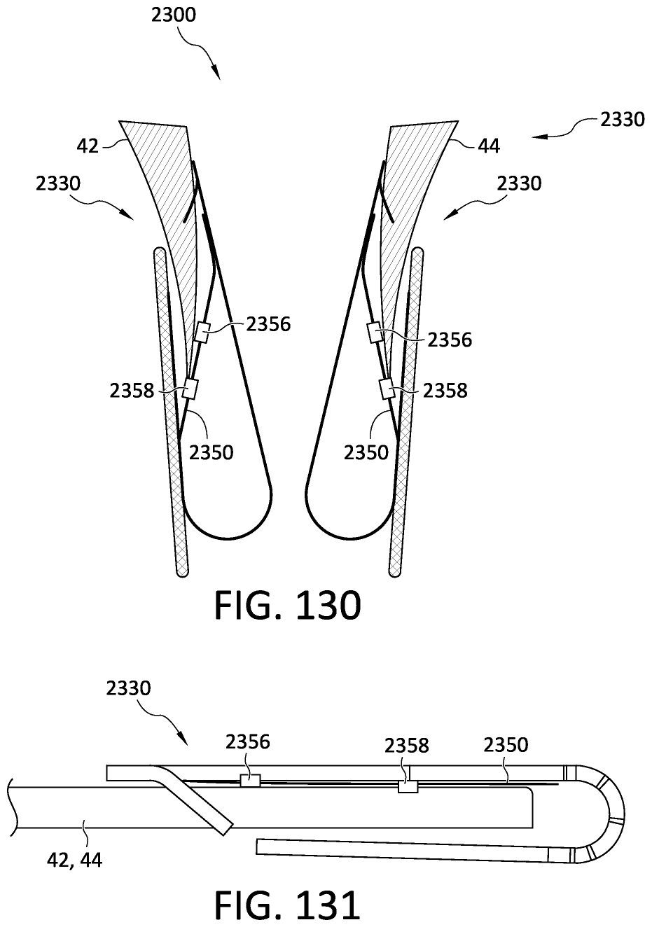

With reference to FIGS. 130-131, the leaflets 42, 44 are repositioned within the clasps 2330 so that they make contact with both the first electrode 2356 and the second electrode 2358. The electrical signals from the first electrode 2356 and the second electrode 2358 can indicate to a user that the leaflets 42, 44 are in an acceptable position and that repositioning of the clasps 2330 is not required.

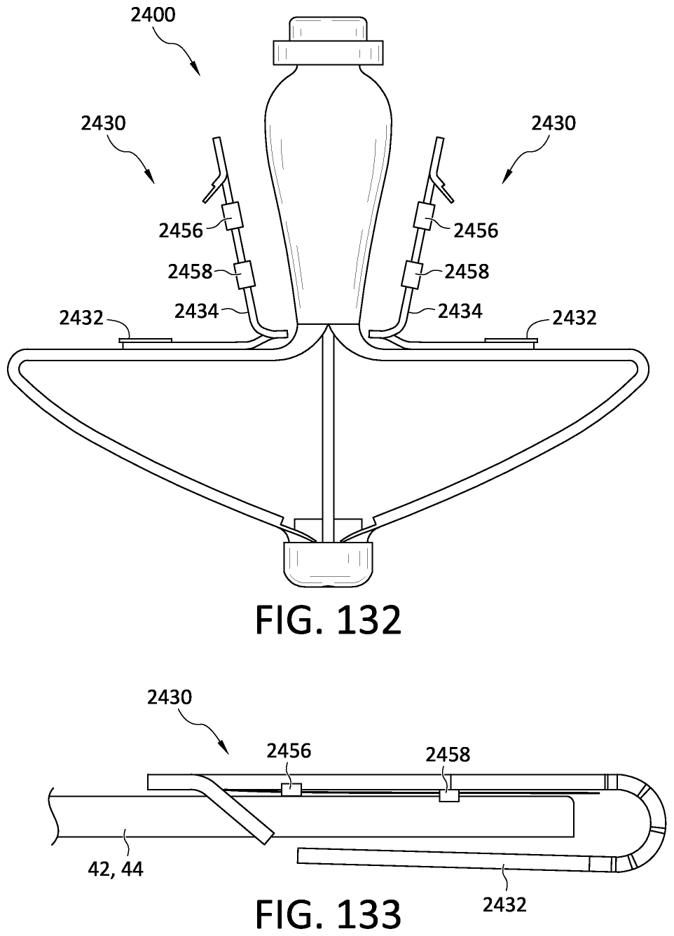

Referring now to FIGS. 132-133, the clasps 2430 of the device 2400 include electrodes on the movable arm 2434. Specifically, the first electrode 2456 and the second electrode 2458 can be coupled at different locations along the movable arm 2434. Alternatively or in addition, electrodes 2456, 2458 can be positioned on a fixed arm 2432 of the clasp 2430 and/or the inner paddle portion of the device. When the clasp is closed, the leaflets engage the electrodes, and the electrical signals from the electrodes can indicate to a user whether the leaflets are in a sufficient position or that repositioning of the clasps is required. In some implementations, IECG signals are measured on the first and second electrodes 2456, 2458. A bipolar signal can be calculated as the signal from the first electrode 2456 subtracted from the signal from the second electrode 2458. The resulting bipolar signal and/or the original signals can provide indications of the first and/or second electrodes 2456, 2458 being in the atrium (and not in contact with tissue), being positioned in blood in the ventricle (and not in contact with tissue), being in contact with valve leaflet tissue and/or being in contact with chordae tissue.

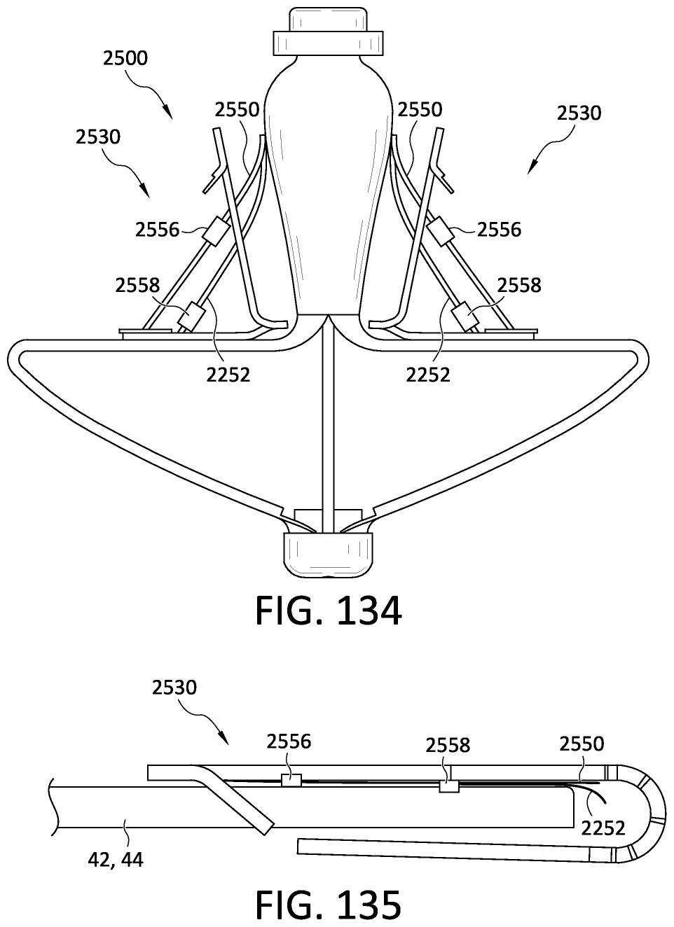

The device can also include multiple indicator arms, with each indicator arm having an electrode for indicating whether the leaflets are in a sufficient position. Referring now to FIGS. 134-135, the device 2500 has a pair of clasps 2530 that each include a first indicator arm 2550 and a second indicator arm 2252. The first indicator arm 2550 includes a first electrode 2556, and the second indicator arm 2252 includes a second electrode 2558. In this instance, when the clasp is closed, the leaflets engage the first electrode 2556 of the first indicator arm 2550 as well as the second electrode 2558 of the second indicator arm 2252, and the electrical signals from the electrodes can indicate to a user whether the leaflets are in a sufficient position or that repositioning of the clasps is required.

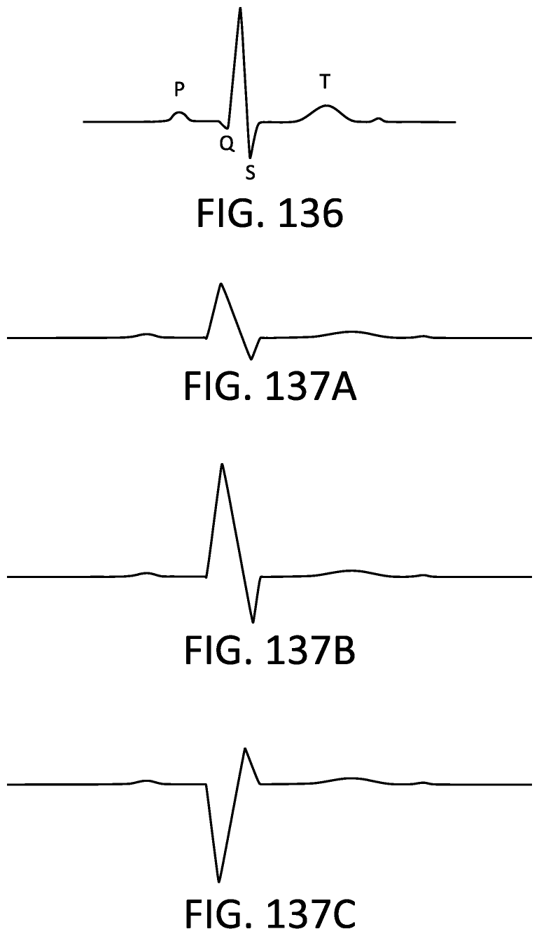

FIG. 136 illustrates an IECG signal reading. The P wave is a small deflection wave that represents atrial depolarization, the Q waves correspond to depolarization of the interventricular septum, the R wave reflects depolarization of the main mass of the ventricles, and the S wave signifies the final depolarization of the ventricles, at the base of the heart.

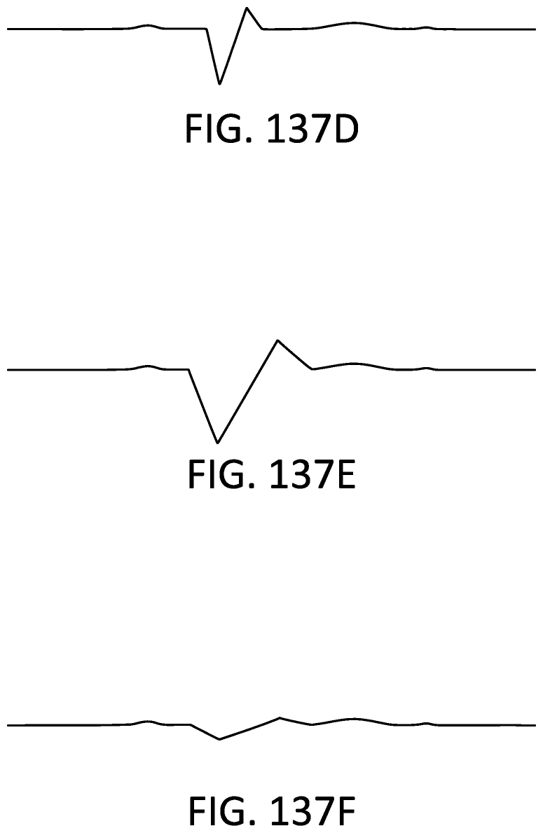

IECG readings from electrodes on a leaflet secured at an adequate depth in a device (e.g., leaflet 42 shown in FIG. 130-131 or 133) are illustrated in FIGS. 137A-137C. FIG. 137A illustrates the waveform signal of electrode 2358 (or 2458) alone. FIG. 137B illustrates the waveform signal of the first electrode 2356 (or 2456) alone. FIG. 137C illustrates the bipolar waveform signal (the waveform signal of FIG. 137A minus the waveform signal of FIG. 137B).

FIG. 137D illustrates the bipolar waveform signal of a leaflet which contacts only the first electrode 2356 (or 2456). For example, this can be the signal provided by the examples illustrated by FIGS. 128-129. The signal from the first electrode would be significantly lower than the signal illustrated by FIG. 137B, because less of the leaflet is inserted into the clasp. This reduced insertion causes a thinner portion of the leaflet to be contacted by the electrode, resulting in the lower amplitude signal. The lack of contact with a leaflet by the electrode 2358 (2458) results in a very low amplitude signal, such as the signal illustrated by FIG. 137F. The bipolar signal illustrated by FIG. 137D and/or individual signals from the two electrodes can be used to determine that the leaflet is inserted to the first electrode 2356 (or 2456), but not as far as the second electrode 2358 (2458). For example, the waveform illustrated by FIG. 137E can correspond to an expected waveform when the leaflet is inserted to the first electrode 2356 (or 2456), but not as far as the second electrode 2358 (2458). Or the set of the bipolar signal and individual signals from the electrodes can correspond to an expected set of waveforms when the leaflet is inserted to the first electrode 2356 (2456), but not to the second electrode 2358 (2458).

FIG. 137E illustrates a bipolar waveform signal where a portion of the chordae tendinea contacts the first electrode 2356 (or 2456). The signal from the first electrode will be different when contacting chordae tendinea than when contacting leaflet tissue. For example, the signal illustrated by FIG. 137E can have a higher amplitude and/or a longer wavelength (i.e., when an electrode contacts the chordae tendinea) than the signal illustrated by FIG. 137C (i.e., when the electrodes contact leaflet tissue). The bipolar signal illustrated by FIG. 137E and/or individual signals from the two electrodes can be used to determine that one or both of the electrodes are in contact with the chorea tendinea. For example, the waveform illustrated by FIG. 137E can correspond to an expected waveform when chordae tendinea is inserted to the first electrode 2356 (or 2456), and the second electrode 2358 (2458) does not contact tissue. Or the set of the bipolar signal and individual signals from the electrodes can correspond to an expected set of waveforms when chordae tendinea contacts the first electrode 2356 (or 2456), but chordae tendinea does not contact second electrode 2358 (2458).

FIG. 137F illustrates the bipolar waveform signal when there is no heart tissue contacting the electrodes. The signal is substantially flat and/or zero, because both sensors are only contacting blood in the heart. The signal can have the shape illustrated by FIG. 137F when the second electrode 2358 (2458) is deeper in the device (e.g., further in the clasp) than the first electrode 2356 (or 2456) and, thus, more shielded. The signal from the electrodes will be different when not contacting tissue (e.g., only contacting the blood in the heart) than when contacting leaflet tissue. For example, the signal illustrated by FIG. 137F can have a lower amplitude and/or be flat or substantially flat. The bipolar signal illustrated by FIG. 137F and/or individual signals from the two electrodes can be used to determine that one or both of the electrodes are in the blood in the heart. The signals from the electrodes will differ when the electrodes (and thus the device) is in the atrium versus when the electrodes are disposed in the ventricle. For example, the signals from each electrode can have a higher magnitude in the ventricle than in the atrium.

Signals from the electrodes can be used to determine a variety of different conditions of the device. For example, the electrodes can be used to determine and/or confirm that the device is disposed in the atrium, is disposed in the ventricle, whether the device is in contact with a leaflet, whether a leaflets is at a sufficient depth in a clasp, whether chordae tendinea is disposed in the device, such as in a clasp, etc.

In some implementations, a portion of the indicator can be formed from the clasp. For example, the indicator can be formed by cutting a portion of the movable arm and shape-setting and/or twisting the cut portion. The indicator can be positioned in a plane such that it can contact a native leaflet and determine whether the clasp has properly engaged the native leaflet.

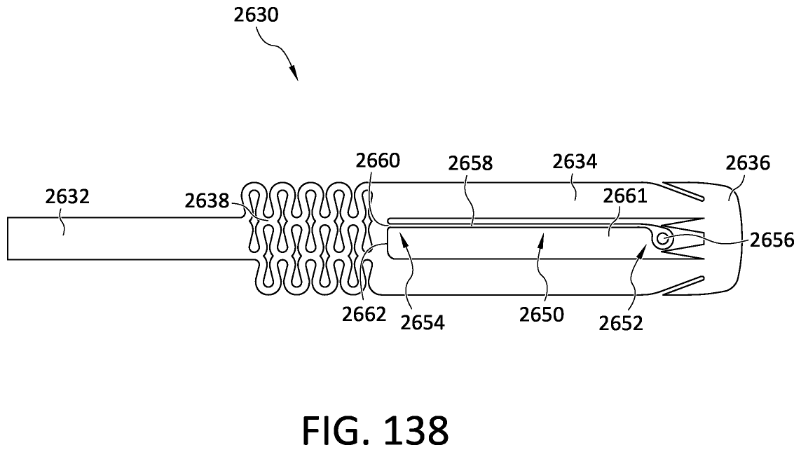

With reference to FIG. 138, flat material 2630 that can be bent to form a clasp and an indicator arm for a valve repair device is illustrated. The flat clasp material 2630 includes a fixed arm 2632, a flex or hinge portion 2638, a movable arm 2634 having a gripping portion 2636 (such as the optional illustrated barbed end), and an indicator arm 2650. The movable arm 2634 can have at least one opening 2661 (e.g., aperture, channel, slot, etc.) in it, which the indicator arm 2650 is configured to pass through. The entire flat clasp material 2630 can be formed from a single piece of flat material.

The indicator arm 2650 is formed from a portion of the movable arm 2634 of the flat clasp material 2630. The indicator arm 2650 can be cut into a portion of the movable arm 2634 by a variety of methods, including laser cutting, etc. The indicator arm 2650 includes a moving end 2652 and a fixed end 2654. The fixed end 2654 of the indicator arm 2650 can be coupled to the movable arm 2634 in a variety of ways and at a variety of locations along the movable arm 2634. In the illustrated example, the movable arm and the indicator arm are cut into the flat clasp material such that the indicator arm remains attached to the movable arm at the junction 2660. The indicator arm 2650 can be coupled to the movable arm 2634 at any point between the hinge portion 2638 and the gripping portion 2636.

The indicator arm 2650 can include an optional indicator marker 2656. In some implementations, the indicator marker 2656 comprises a radiopaque material that can be printed or attached as a separate piece of material to the indicator marker 2656. For example, the radiopaque material can be a coil made of platinum or another radiopaque material. The indicator marker is not necessarily a separate component. For example, in some implementations, the indicator marker 2656 is integral with the indicator arm, e.g., the indicator marker 2656 can be portion of the indicator arm that comprises radiopaque material and/or is thicker or has a larger surface area (which can help increase visibility).

The indicator marker 2656 can be visible with fluoroscopy and/or other imaging techniques and can assist the user in determining whether the leaflet is properly positioned in the clasp 2630. The indicator arm 2650 can be used with a suitable valve repair device, such as any of the valve repair devices disclosed herein (see also, for example, the valve repair devices disclosed by Published PCT application WO 2020/168081, which is incorporated herein by reference in its entirety).

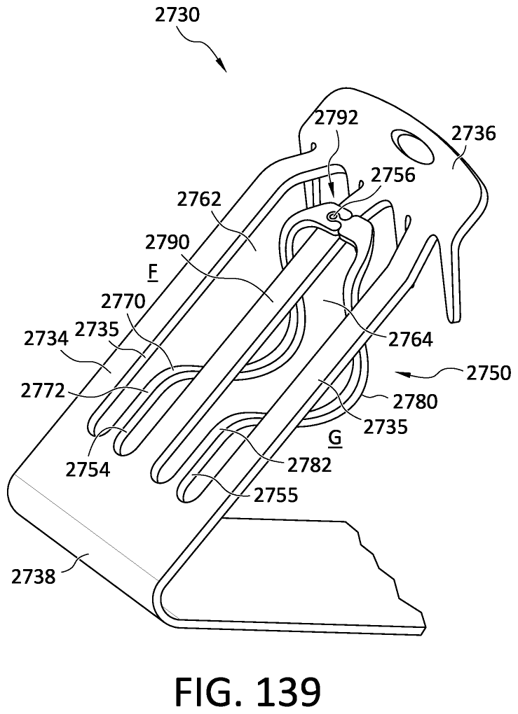

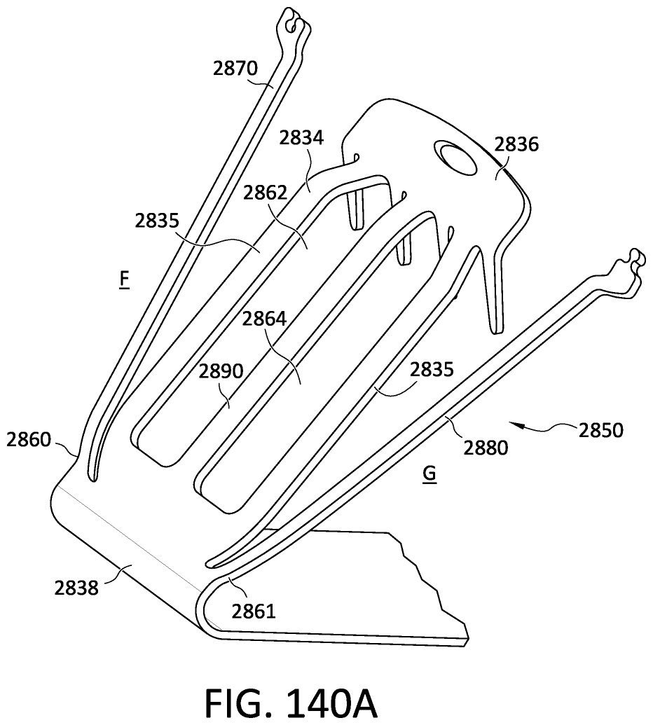

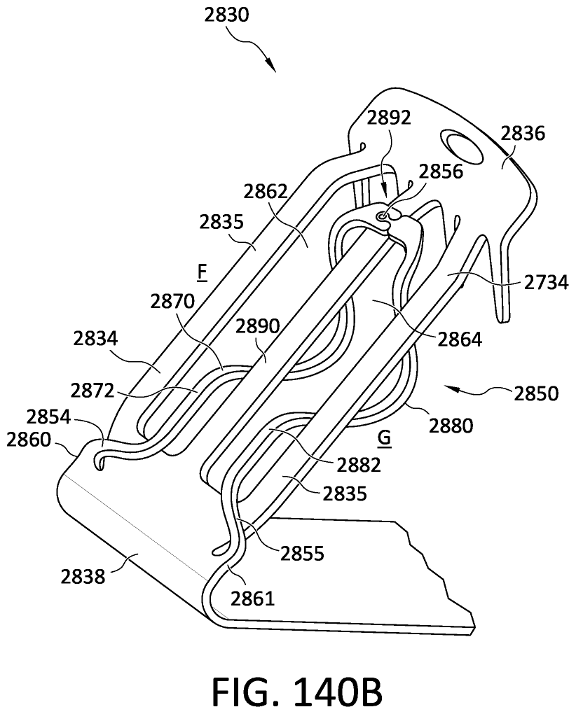

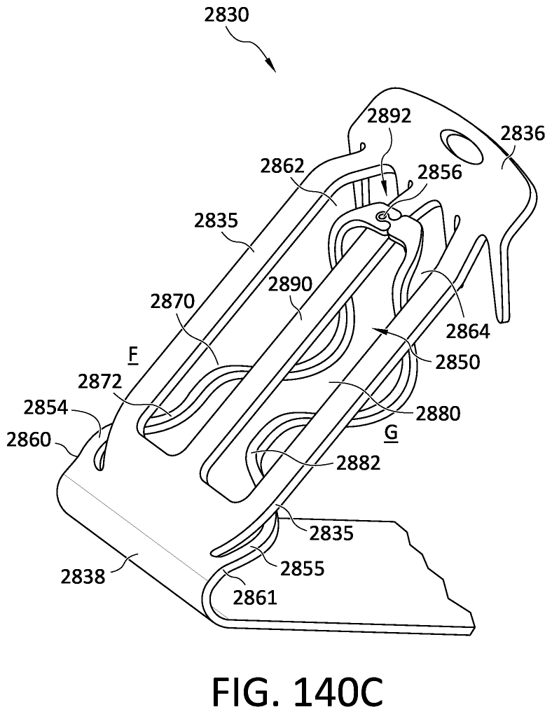

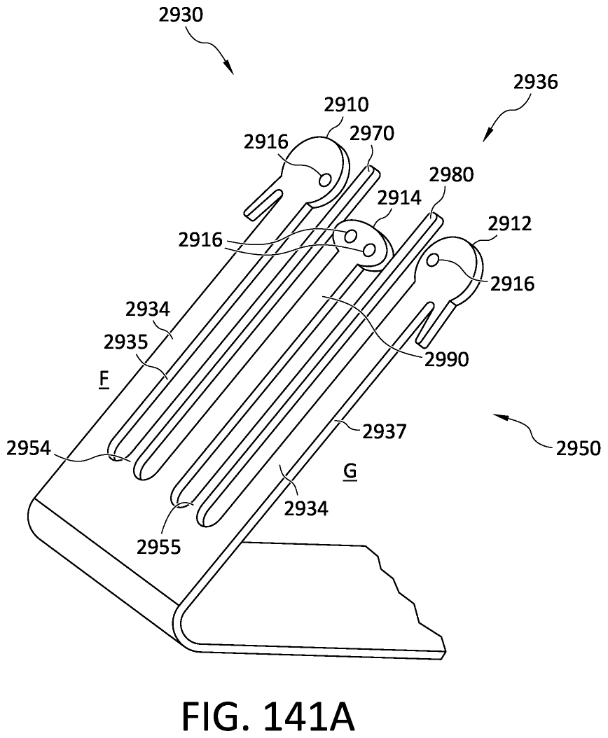

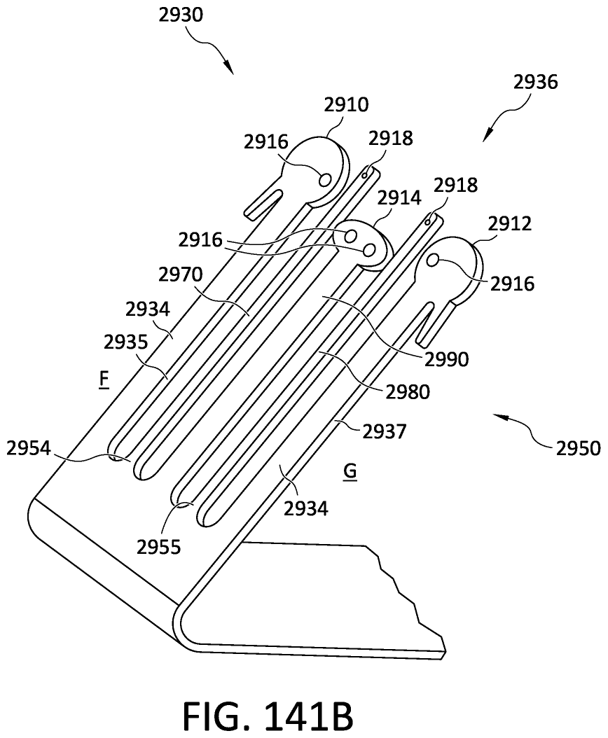

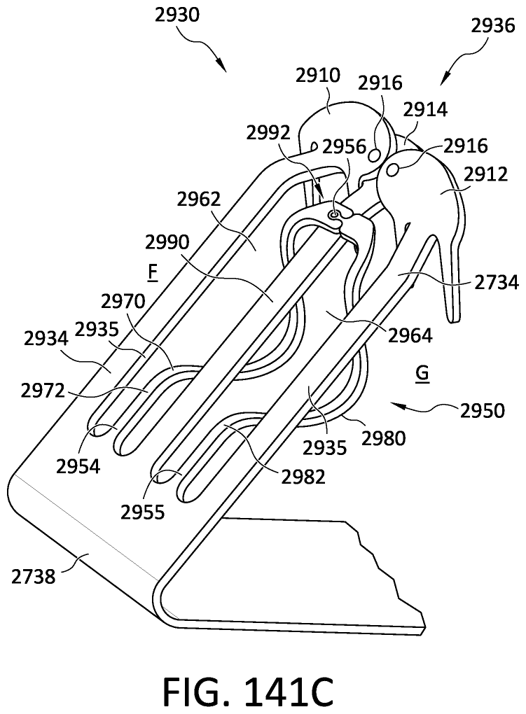

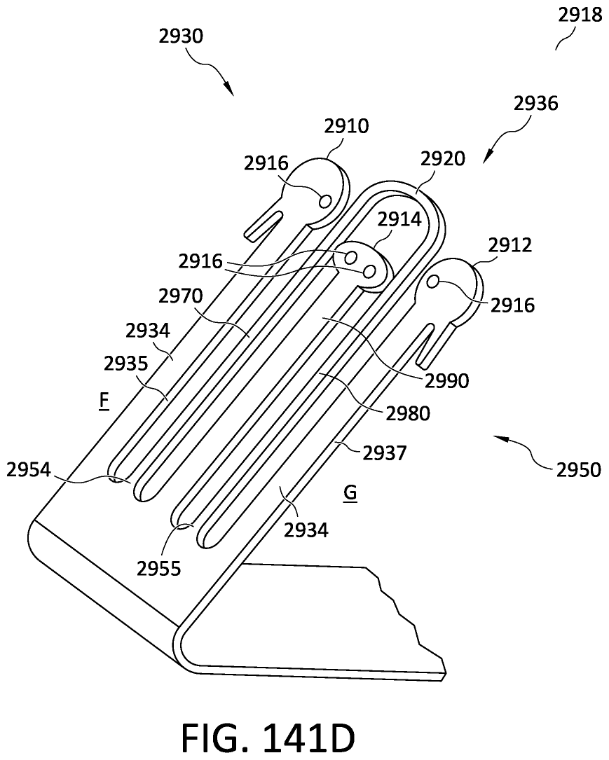

The indicator arm 2650 meets the movable arm 2634 at a junction 2660 on the movable arm 2634. The junction 2660 can be located at various positions along the movable arm 2634. For example, the junction 2660 can be located at a base 2662 on the movable arm 2634 at a position near the hinge portion 2638. The junction 2660 can also be located along a side of an interior edge (see FIG. 139 and FIGS. 141A-B) or exterior edge (see FIGS. 140A-C) of the movable arm at any point between the hinge portion 2638 to the gripping portion 2636. In another implementation, the junction 2660 can be at the gripping portion 2636 end of the movable arm and extend toward the base 2662.

The indicator arm 2650 can be a variety of lengths. In some implementations, the indicator arm 2650 is cut along a length of the movable arm 2634 from the hinge portion 2638 to the gripping portion 2636. In other implementations, the indicator arm 2650 extends along only a portion of the movable arm 2634 between the hinge portion 2638 to the gripping portion 2636. In some implementations, the length of the indicator arm is between 2.0 mm and 15.0 mm including any subrange, including between 5.0 mm and 10.0 mm, and between 6.0 mm and 8.0 mm.

The indicator arm 2650 can have a range of thicknesses. In some implementations, the thickness of the indicator arm is between 0.100 mm and 0.500 mm, including between 0.250 mm and 0.400 mm, and between 0.320 mm and 0.380 mm. In some implementations, the thickness of the indicator arm is 0.380 mm. The indicator arm can have thickness in subranges of any of these ranges.

The indicator arm 2650 can have a range of widths. In some implementations, the width of the indicator arm is between 0.025 mm and 0.250 mm, including between 0.040 mm and 0.120 mm, and between 0.075 mm and 0.100 mm. In some implementations, the thickness of the indicator arm is 0.050 mm. The indicator arm can have widths that are in subranges of any of these ranges. In an example implementation, by cutting both the movable arm end the indicator arm from a single, single thickness, material, the relative flexibilities of the indicator arm and the movable arm can be controlled by selecting the relative widths of the portions of material that form the movable arm and the portion of material that forms the indicator arm.

In some implementations, the indicator arm can be bent to include one or more twisted portions between the moving end and the fixed end of the indicator arm. With reference to FIG. 138, the indicator arm 2650 can include a twisted portion 2658 between the moving end 2652 and the fixed end 2654. The twisted portion can include one or more twists, with each twist ranging from 0 degrees to 180 degrees, relative to the untwisted portion of the indicator arm. In some implementations, the twisted portion can be twisted between 5 degrees and 170 degrees, between 15 degrees and 145 degrees, between 30 degrees and 120 degrees, or between 60 degrees and 90 degrees. In some implementations, the twisted portion is twisted 90 degrees relative to the untwisted portion. The twists can be clockwise or counterclockwise relative to the untwisted portion. The twist of the twisted portion can cause the moving end of the indicator arm to be positioned between the movable arm 2634 and the fixed arm 2632 of the clasp 2630 (for example, at the second side G, as illustrated in FIGS. 139, 140B, 140C, and 141C. The twists cause the moving end 2652 to be positioned such that the indicator arm 2650 can contact a native leaflet when inserted into the clasp 2630.

In some implementations, the twist of the indicator arm is configured to make the indicator arm flex more easily (or less easily). For example, when the indicator arm is narrower than the thickness of the indicator arm, an indicator arm that is bent 90 degrees will flex more easily when engaged by leaflet tissue than an indicator arm that is not bent. As a result, the flexibility or responsiveness of the indicator arm can be controlled with the width of the indicator arm and by twisting the indicator arm.

FIG. 139 illustrates an example implementation of a clasp 2730 with an indicator arm 2750 that can be made from a single piece of flat material. The indicator arm 2750 of the clasp 2730 can include a first arm portion 2770 and a second arm portion 2780. Both the first arm portion 2770 and the second arm portion 2780 can be similar in various regards (including length, width, and thickness) to the indicator arm 2650 of FIG. 138. In some implementations, the movable arm 2734 includes a center beam 2790 and two outer beams 2735 disposed between the hinge portion 2738 and the gripping portion 2736 (such as the optional illustrated barbed end). The center beam 2790 and the two outer beams define the size of the openings 2762, 2764.

The first arm portion 2770 and second arm portion 2780 are each cut from the material between the center beam 2790 and the two outer beams 2735 of the movable arm 2734. The material between the center beam 2790 and the two outer beams 2735 can be straightened, stretched, bent, or otherwise processed or treated to make the indicator arm 2735. For example, the material between the center beam 2790 and the two outer beams 2735 of the indicator arm portions 2770, 2780 can be cut in a tortuous path to extend the length of the material that forms the indicator arm portion and straighten, bend, or otherwise treat the material to form the indicator arm portions 2770, 2780 shown in FIG. 139. The first arm portion 2770 includes a bent portion 2772 adjacent to the fixed end 2754 of the first arm portion 2770. The second arm portion 2780 also includes a bent portion 2782 adjacent to the fixed end 2755 of the second arm portion 2780.

The bent portions 2772, 2782 cause the first and second arm portions 2770, 2780 to extend to the second side G of the clasp 2730, where the indicator arm 2650 can contact a native leaflet when inserted into the clasp 2730. The first arm portion 2770 and the second arm portion 2780 can be connected at a connection point 2792 on the first side F of the clasp 2830. The first arm portion 2770 and the second arm portion 2780 can be connected by various means, including by welding, press fitting, etc. In some implementations, the indicator arm 2750 includes an indicator marker 2756, similar in material aspects to indicator marker 2656. In some implementations, the first arm portion 2770 is secured to the second arm portion 2780 with the indicator marker 2756, which is press fit into both the first arm portion 2770 and the second arm portion 2780. The axis of indicator marker 2756 can be press fit in the first arm portion 2770 and the second arm portion 2780 in a space formed by the connection at the ends of the indicator arm, a stacked configuration, or a mirrored configuration. The stacked configuration can position the axis of the indicator marker 2756 perpendicular to the plane of the indicator arm 2750, whereas the mirrored configuration puts the axis of the indicator marker 2756 within the plane of the indicator arm 2750. Depending on the dimensions of the indicator marker 2756 used, different orientations can be more visible to the available fluoroscopy angles.

FIGS. 140A-140C illustrate example implementations of clasps with integral indicator arms 2850, where the indicator arms are formed from material that is outside the outer beams 2835. In these examples, the first arm portion 2870 and second arm portion 2880 of the indicator arm 2850 are formed from flat material that is disposed laterally relative to the flat material that the outer beams 2835 of the movable arm 2834 are made from. Both the first arm portion 2870 and the second arm portion 2880 can be similar in various regards (including length, width, and thickness) to the indicator arm 2650 of FIG. 138, except the arm portions 2870, 2880 are made from material on the outside of the clasp.

With reference to FIGS. 140A-140C, the first arm portion 2870 can meet the movable clasp arm 2834 at a first junction 2860 on the movable arm 2834. The second arm portion 2880 meets the movable arm 2834 at a second junction 2861 on the movable arm 2834. The junctions 2860, 2861 can be located at various positions along the movable arm 2834. For example, the junctions 2860, 2861 can be located on the movable arm 2834 at a position near the hinge portion 2838. The junctions 2860, 2861 can also be located along the outer beams 2835 of the movable arm 2834 at any point between the hinge portion 2838 to the gripping portion 2836 (such as the optional illustrated barbed end). FIG. 140A illustrates the first arm portion 2870 and the second arm portion 2880 of the indicator arm 2850 after they are formed from the material adjacent to the outer beams 2835 of the movable arm 2834, but before they are shaped to form the indicator arm 2850.

With reference to FIGS. 140B and 140C, the first arm portion 2870 and the second arm portion 2880 can be configured in a variety of ways. With reference to FIG. 140B, the first arm portion 2870 includes a twisted and/or bent portion 2872 adjacent to the fixed end 2854 of the first arm position 2870. The second arm portion 2880 also includes a twisted and/or bent portion 2882 adjacent to the fixed end 2855 of the second arm portion 2880. In the FIG. 140B implementation, the twisted and/or bent portions 2872, 2882 are configured such that the first and second indicator arm portions 2870, 2880 extend across the outer beams 2835 on the side F. Then, the first and second indicator arm portions 2870, 2880 extend through the spaces 2862, 2864 between the outer beams 2835 and the center beam 2890 to the second side G of the clasp 2830, where the indicator arm 2850 can contact a native leaflet when inserted into the clasp 2830. The first and second indicator arm portions can include additional twists and/or bends to allow the first arm portion 2870 and the second arm portion 2880 can be connected at a connection point 2892 on the first side F of the clasp 2830. The first arm portion 2870 and the second arm portion 2880 can be connected by various means, including by welding, etc. In some implementations, the indicator arm 2850 can include an indicator marker 2856, similar in material aspects to indicator marker 2656, 2756.

With reference to FIG. 140C, the first arm portion 2870 includes a twisted and/or bent portion 2872 adjacent to the fixed end 2854 of the first arm position 2870. The second arm portion 2880 also includes a twisted and/or bent portion 2882 adjacent to the fixed end 2855 of the second arm portion 2880. In the FIG. 140C implementation, the twisted and/or bent portions 2872, 2882 are configured such that the first and second indicator arm portions 2870, 2880 extend across the outer beams 2835 on the inner side G. The first and second indicator arm portions 2870, 2880, are bent in a configuration where the indicator arm 2850 can contact a native leaflet when inserted into the clasp 2830. The first and second indicator arm portions can include additional twists and/or bends to 2880 to extend through the spaces 2862, 2864 between the outer beams 2835 and the center beam 2890 to the side F of the clasp 2830 and can be connected at a connection point 2892. The first arm portion 2870 and the second arm portion 2880 can be connected by various means, including by welding, etc. In some implementations, the indicator arm 2850 can include an indicator marker 2856, similar in material aspects to indicator marker 2656, 2756.

Clasps with integral leaflet depth indicator(s) can be made from a single piece of flat material in a wide variety of different ways. With reference to FIGS. 141A-141B, in some implementations, the gripping portion 2936 of the clasp 2930 can include a first gripping member 2910, a second gripping member 2912, and a third gripping member 2914. The gripping members can be the same as or similar to other gripping members, clasps, clasp arms, etc. described elsewhere herein). The first gripping member 2910 and second gripping member 2912 can each connect to the third gripping member 2914 by way of connecting members 2916. The connecting members 2916 can take a wide variety of different forms. For example, the connecting members 2916 can comprise sutures, fasteners, pins, snaps, magnets etc. In some implementations, the connecting members can extend through openings in one or more of a first gripping member 2910, a second gripping member 2912, and a third gripping member 2914.

Configuring the first gripping member 2910, the second gripping member 2912, the third gripping member 2914, the first indicator arm portion 2970, and/or the second indicator arm portion 2980 in the manner illustrated by FIGS. 141A and 141B can facilitate easier manufacturing of the clasps 2930 with integral leaflet depth indicator(s). For example, in the implementations illustrated by FIGS. 141A and 141B, the first indicator arm portion 2970 and the second indicator arm portion 2980 extend past the first gripping member 2910, the second gripping member 2912, and the third gripping member 2914. This can allow the leaflet depth indicator to be longer then would be possible if the arm or arms were formed only from material in a window of the movable arm.

The clasps 2930 illustrated by FIGS. 141A and 141B are similar, except the indicator arm portions 2970, 2980 of FIG. 141A do not include connecting members and the indicator arm portions of FIG. 141B include connecting members 2918. In the FIG. 141A implementation, the indicator arm portions 2970, 2980 will not be attached to one another and can form two independently movable leaflet depth indicators. The inclusion of two side by side leaflet depth indicators can provide additional information about the position of the leaflet relative to the clasp. For example, two side by side leaflet depth indicators can provide an indication of rotation and/or offset of the clasp relative to the leaflet in addition to the depth of the leaflet in the clasp. Two independent leaflet depth indicator arms can be used in any of the implementations disclosed herein.

In the FIG. 141B implementation, the first indicator arm portion 2970 can connect to the second indicator arm portion 2980 by way of connecting members or features 2918. The connecting members or features 2918 can take a wide variety of different forms. For example, the connecting members or features 2918 can comprise complementary sutures, fasteners, pins, snaps, magnets etc. In some implementations, the connecting members can extend through openings in one or more of the first indicator arm portion 2970 and the second indicator arm portion 2980. In some implementations, the first indicator arm portion 2970 can connect to the second indicator arm portion 2980 by other means. For example, with reference to FIG. 141C, the first indicator arm portion 2970 can connect to the second indicator arm portion similar to the first indicator arm portion and second indicator arm portion of FIG. 140C.

With reference to FIGS. 141A and 141B, the gripping portion 2936 (such as the optional illustrated barbed end) is illustrated in a preliminary configuration, whereby the first gripping member 2910 and second gripping member 2912 are not yet connected to the third gripping member 2914. With reference to FIG. 141C, the gripping portion 2936 is illustrated in a formed or assembled configuration, whereby the first gripping member 2910 and second gripping member 2912 are connected to the third gripping member 2914 by way of the corresponding connection members 2916.—

With reference to FIG. 141C, the first arm portion 2970 includes a twisted and/or bent portion 2972 adjacent to the fixed end 2954 of the first arm position 2970. The second arm portion 2980 also includes a twisted and/or bent portion 2982 adjacent to the fixed end 2955 of the second arm portion 2980. The first and second indicator arm portions 2970, 2980 extend on the second side G of the spaces 2962, 2964 between the outer beams 2835 and the center beam 2990, where the indicator arm 2950 can contact a native leaflet when inserted into the clasp 2930. The first and second indicator arm portions can include additional twists and/or bends to allow the first arm portion 2970 and the second arm portion 2980 can be connected at a connection point 2992 on the first side F of the clasp 2930. The first arm portion 2970 and the second arm portion 2980 can be connected by various means, including by welding, etc. In some implementations, the indicator arm 2950 can include an indicator marker 2956.

With reference to FIG. 141D, the first arm portion 2970 can have a bent portion 2972 adjacent to the fixed end 2954 of the first arm position 2970. The second arm portion 2980 can also include a bent portion 2982 adjacent to the fixed end 2955 of the second arm portion 2980. When bent, the first and second indicator arm portions 2970, 2980 extend on the second side G of the spaces 2962, 2964 between the outer beams 2934 and the center beam 2990, where the indicator arm 2950 can contact a native leaflet when inserted into the clasp 2930. The first and second indicator arm portions 2970, 2980 are integrally formed with a transition portion 2920. The first and second indicator arm portions 2970, 2980 can include additional twists and/or bends to allow the transition portion 2920 to be positioned on the first side F of the clasp 2930. In some implementations, the transition portion 2920 can include an indicator marker.

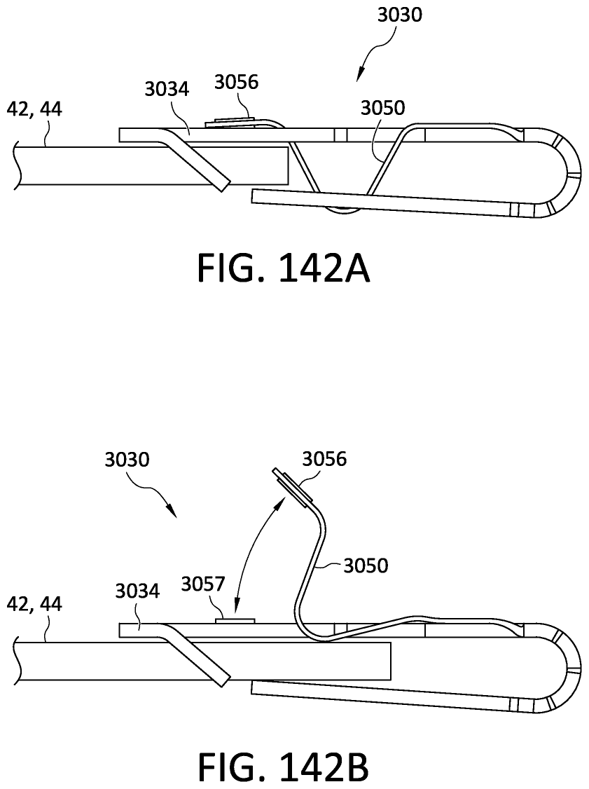

With reference to FIGS. 142A-142B, a clasp 3030 is illustrated in a closed position. In some implementations, the clasp 3030 is the same or substantially similar to any of clasps 2630, 2730, 2830, or 2930. Referring to FIG. 142A, the leaflet is located within the clasp 3030, but is not located far enough within the clasp 3030 to engage the indicator arm 3050. The optional indicator marker 3056 (when included) therefore does not move away from the movable arm 3034 of the clasp 3030 or the optional indicator marker 3057 (when included). The position of the indicator marker 3056 and/or the indicator marker 3057, which can be visible with fluoroscopy and/or other imaging techniques, can assist the user in determining that the leaflet 42, 44 is not properly positioned in the clasp 3030. For example, when both indicator markers 3056, 3057 are included, an image (e.g., fluoroscopy image) that shows only a single marker (i.e., the two markers 3056, 3057 are adjacent to one another or abutting one another and only a single mass can be seen on the image) indicates that tissue, such as valve leaflet tissue is not disposed in the clasp 3030 to a sufficient depth.

Referring to FIG. 142B, the leaflet is located far enough within the clasp 3030 to engage the indicator arm 3050. The movement of the indicator arm 3050 results in the movement of the indicator marker 3056 from the movable arm 3034 of the clasp 3030. The position of the indicator marker 3056, which can be visible with fluoroscopy and/or other imaging techniques, can assist the user in determining that the leaflet 42, 44 is properly positioned in the clasp 3030. For example, when both indicator markers 3056, 3057 are included, an image (e.g., fluoroscopy image) that shows two separate markers (i.e., the two markers 3056, 3057 are spaced apart) indicates that tissue, such as leaflet tissue is disposed in the clasp 3030 to a sufficient depth.

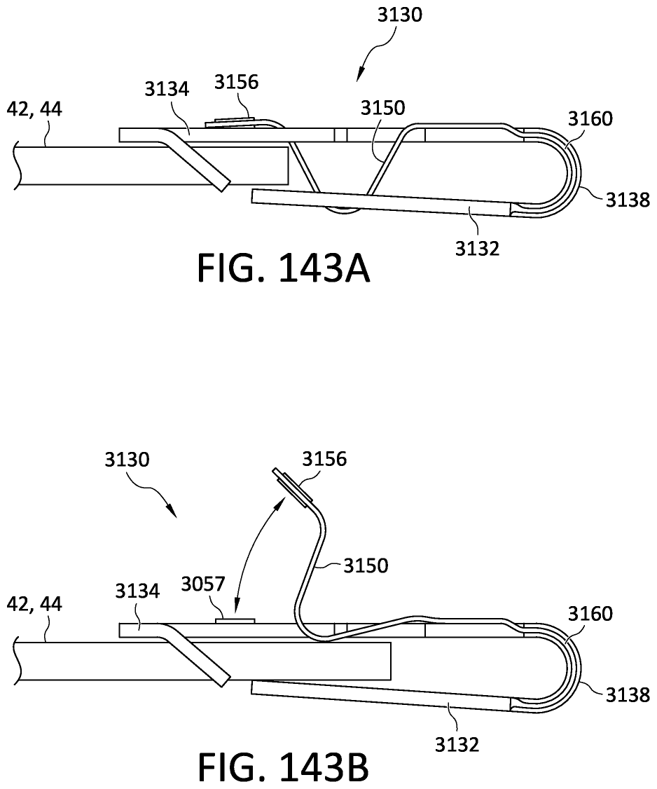

In the implementations illustrated by FIGS. 138, 139, 140A-140C, 141A-141D, 142A-142B, the leaflet depth indicators 2650, 2750, 2850, 2950 extend from the movable arms of the clasp. However, in other implementations, the leaflet depth indicator can extend from the hinge portion or the fixed arm portion of the clasp. For example, the clasp 3130 in the implementation illustrated by FIGS. 143A and 143B, a leaflet depth indicator 3150 extends from the fixed arm 3132 of the clasp. The leaflet depth indicator 3150 can be integrally formed with the clasp 3130. In the implementation illustrated by FIGS. 143A and 143B, the leaflet depth indicator 3150 originates at the fixed arm 3132 As is shown in FIGS. 143A and 143B, the leaflet depth indicator 3150 includes a curved portion 3160 that extends along the hinge portion 3138. The leaflet depth indicator then extends along the movable arm 3134 of the clasp 3130.

Referring to FIG. 143A, the leaflet is located within the clasp 3030, but is not located far enough within the clasp 3130 to engage the indicator arm 3150. The indicator marker 3156 therefore does not move from the movable arm 3134 of the clasp 3130. The position of the indicator marker 3156, which can be visible with fluoroscopy and/or other imaging techniques, can assist the user in determining that the leaflet 42, 44 is not properly positioned in the clasp 3030. For example, when both indicator markers 3156, 3157 are included, an image (e.g., fluoroscopy image) that shows only a single marker (i.e., the two markers 3156, 3157 are adjacent to one another or abutting one another and only a single mass can be seen on the image) indicates that tissue, such as leaflet tissue is not disposed in the clasp 3130 to a sufficient depth.

Referring to FIG. 143B, the leaflet is located far enough within the clasp 3130 to engage the indicator arm 3150. The movement of the indicator arm 3150 results in the movement of the indicator marker 3156 from the movable arm 3134 of the clasp 3130. The position of the indicator marker 3156, which can be visible with fluoroscopy and/or other imaging techniques, can assist the user in determining that the leaflet 42, 44 is properly positioned in the clasp 3030. For example, when both indicator markers 3156, 3157 are included, an image (e.g., fluoroscopy image) that shows two separate markers (i.e., the two markers 3156, 3157 are spaced apart) indicates that tissue, such as leaflet tissue is disposed in the clasp 3130 to a sufficient depth.

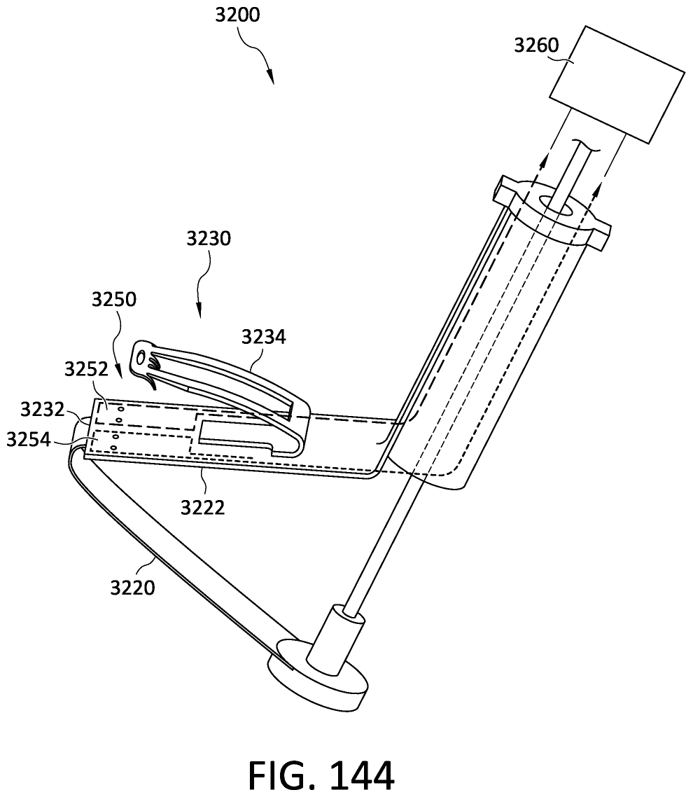

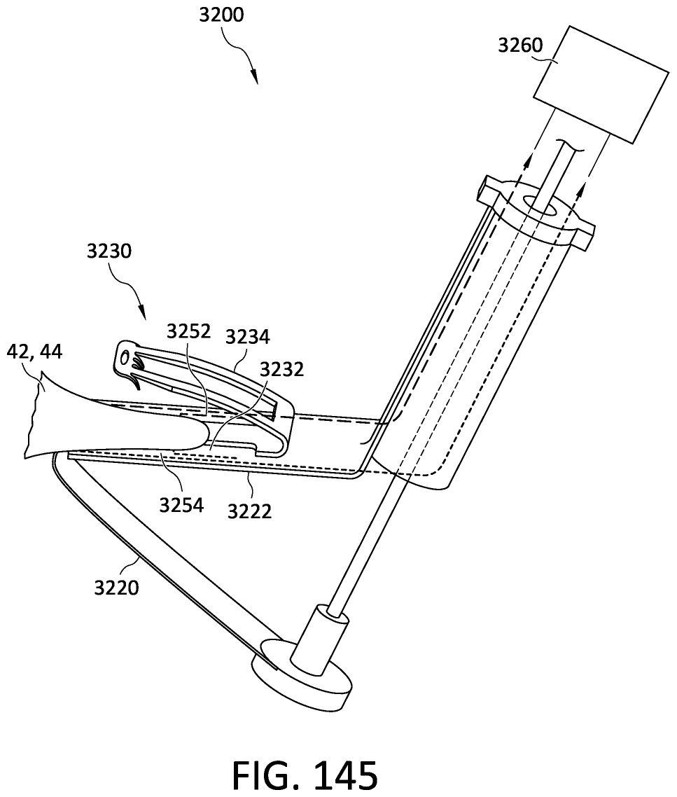

FIGS. 144-145 illustrate an implementation of a device 3200 with a leaflet indicator 3250. The leaflet indicator 3250 can be used with a variety of different devices 3200. For example, the leaflet indicator 3250 can be used with any of the valve repair devices disclosed herein or any other valve repair device. In the illustrated example, the device 3200 includes an inner paddle 3222 and an outer paddle 3220, as well as a clasp 3230 comprising a movable arm 3234 and a fixed arm 3232. The leaflet indicator 3250 can be coupled to various components on the device, such as the inner paddle 3222, the fixed arm of the clasp 3232, and/or the movable arm 3234 of the clasp 3230. In the illustrated example, the leaflet indicator 3250 is disposed on the inner paddle 3222.

The indicator 3250 can take a wide variety of different forms. For example, the indicator 3250 can comprise one or more components capable of sensing an electrical characteristic of a material, such as blood or tissue, which can be a valve leaflet, chordae tendinea, papillary muscles, heart wall tissue, etc. and/or contact by a valve repair device component, such as a class arm, a paddle portion, a coaptation element, etc. In the illustrated example, the indicator 3250 can include one or more electrically conductive contacts, for example, a first contact 3252 and a second contact 3254. Although two indicator contacts are illustrated in FIGS. 144-145, any number of indicator contacts can be used for the indicator. The indicator contacts can be electrically coupled to one or more sensors. The sensors can be coupled with the indicator contacts through multiple ways, including electrically conductive wiring. The sensors can include electrical sensors which can measure one or more of resistance, inductance, capacitance, voltage, current and impedance.

With reference to FIG. 144, a leaflet is not positioned in the clasp 3230. If the clasp 3230 closes without a leaflet positioned between the movable arm 3234 and the fixed arm 3232, the moveable arm 3234 can move and make contact with the indicator 3250 and create a bridge between the first indicator contact 3252 and the second indicator contact 3254. In this instance, the sensor 3260 senses the lack of resistance (e.g., the circuit is closed by the moveable clasp arm). This information can be used to determine that the leaflet is not present in the clasp 3230.

With reference to FIG. 145, a leaflet 42, 44 is positioned in the clasp 3230. If the clasp 3230 closes with the leaflet 42, 44 positioned between the movable arm 3234 and the fixed arm 3232, the moveable arm 3234 may not make contact with the indicator 3250 when it bends to a closed position. In this instance, the sensor 3260 can observe or otherwise indicate that there is a measurable resistance between the contacts 3252, 3254, which can be used to determine that the leaflet is present in the clasp 3230.

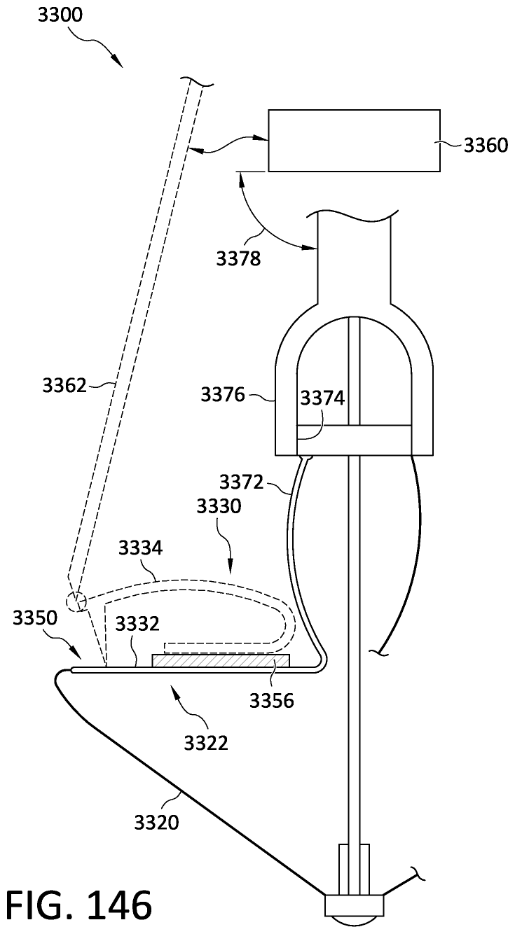

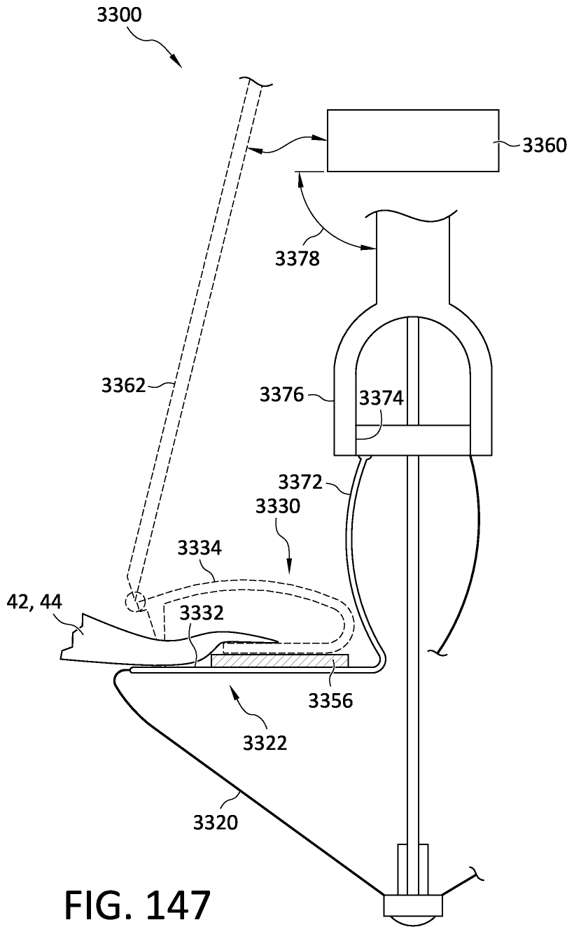

With reference to FIG. 146-147, an implementation of a system 3301 having a leaflet indicator 3350 is illustrated. In this implementation, components of the valve repair system itself are used as the indicator 3350. A wide variety of different configurations of valve repair system components can be used as a leaflet depth indicator. The illustrated device 3300 includes an inner paddle 3322 and an outer paddle 3320 as well as a clasp 3330 comprising a movable arm 3334 and a fixed arm 3332. In the illustrated example, and insulator 3356 is positioned between the inner pedal 3322 and the fixed arm 3332. The device 3300 can include any device disclosed herein, as well as any other valve repair device. The leaflet indicator 3350 can comprise various components on the device that can be electrically coupled to a proximal control handle (not shown). In the illustrated example, a first electrical path is defined by the control line 3362 and the clasp 3330. A second electrical path is defined by the inner panel 3322, the coaptation element 3372, and the coupler 3376. The electrical paths can be formed in a wide variety of different ways. For example, components, portions of the components, or auxiliary components that run along the components can be formed from an electrically conductive material.

The indicator 3350 can be electrically coupled to one or more sensors 3360. The sensors can include electrical sensors which can measure one or more of resistance, inductance, capacitance, voltage, current, impedance, etc. The sensor 3360 can be coupled with the indicator 3350 through multiple ways. The indicator 3350 can be electrically coupled to the sensor 3360 through the first path defined by the control line 3362 and the clasp 3330 and the second path defined by the inner panel 3322, the coaptation element 3372, and the coupler 3376. In some implementations, the device is made from electrically conductive components. For example, the moveable arm, the coaptation element 3372, the collar 3374, the catheter coupler 3376, and/or the actuation lines 3378 can be electrically conductive.

With reference to FIG. 146, a leaflet is not positioned in the clasp 3330. If the clasp 3330 closes without a leaflet positioned between the movable arm 3334 and the fixed arm 3332, the moveable arm 3334 can move and make contact with the indicator 3350 thereby closing the circuit between the first path, the sensor 3360, and the second path. In this instance, the sensor 3360 indicates the lack resistance (e.g., the circuit is closed by the moveable clasp arm), that can be used to determine that the leaflet is not present in the clasp 3330.

With reference to FIG. 147, a leaflet 42, 44 is positioned in the clasp 3330. If the clasp 3330 closes with the leaflet 42, 44 positioned between the movable arm 3334 and the fixed arm 3332, the moveable arm 3334 does not make contact with the inner paddle 3322. In this instance, the circuit between the sensor 3360, the first path, and the second path is interrupted (open) and the sensor 3360 can determine that the leaflet is present in the clasp 3330.

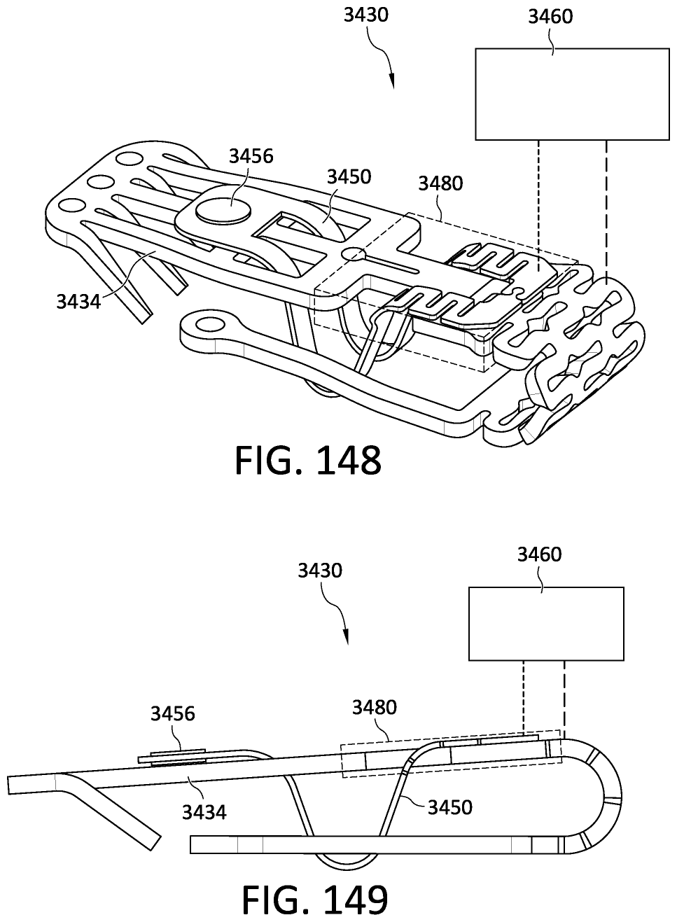

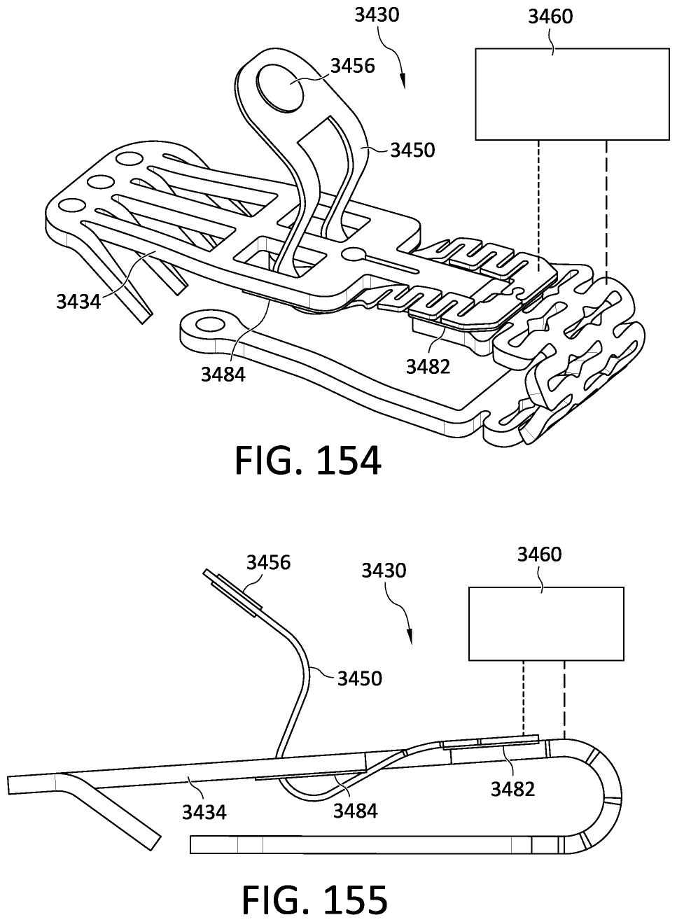

With reference to FIGS. 148-155, in some implementations a visual indicator 3450 is coupled to the movable arm 3434 of the clasp 3430 and the visual indicator 3450 and the clasp 3430 act as an electrical indicator. The visual indicator 3450 and the clasp 3430 can take a variety of different forms. For example the indicator 3450 and the clasp 3430 can be any of the clasps and indicators disclosed in this patent application. In the implementations illustrated by FIGS. 148-155 the visual indicator 3450 can be in accordance with FIGS. 94-98. A circuit can be formed by a sensor 3460, the clasp 3430, and the visual indicator 3450, via wiring connecting the clasp 3430 and the visual indicator 3450 to the sensor 3460.

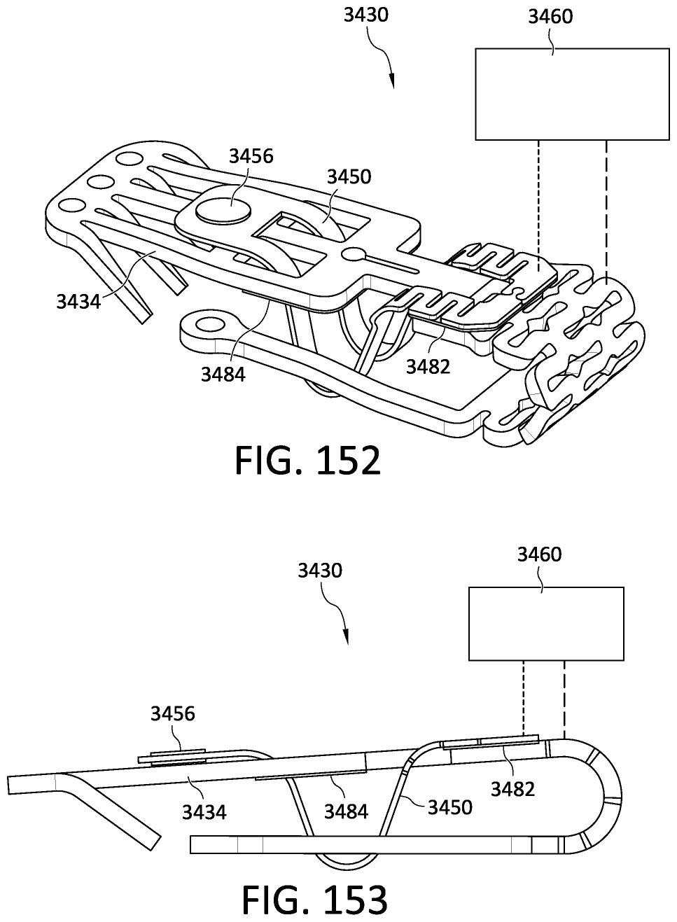

Referring to FIGS. 148-151, in some implementations an insulator 3480 insulates one or more portions of the indicator 3450 from the clasp 3430. The insulator 3480 can take a variety of different forms. In the example of FIGS. 148-151, the portions of the indicator 3450 and the clasp 3430 that are electrically insulated from one another are schematically illustrated by dashed line region 3480. The schematically illustrated insulator 3480 can be achieved in a variety of different ways. With reference to FIGS. 152-155, the indicator 3450 and the clasp 3430 are electrically insulated from each other through one or more insulating components, for example first insulator 3482 and second insulator 3484. The first insulator 3482 insulates the visual indicator 3450 and the clasp 3430 at the connection between the clasp and the indicator. The second insulator 3484 insulates the crossbar of the clasp 3430 from the curved portion of the visual indicator when the visual indicator 3450 is in the leaflet engaged position.

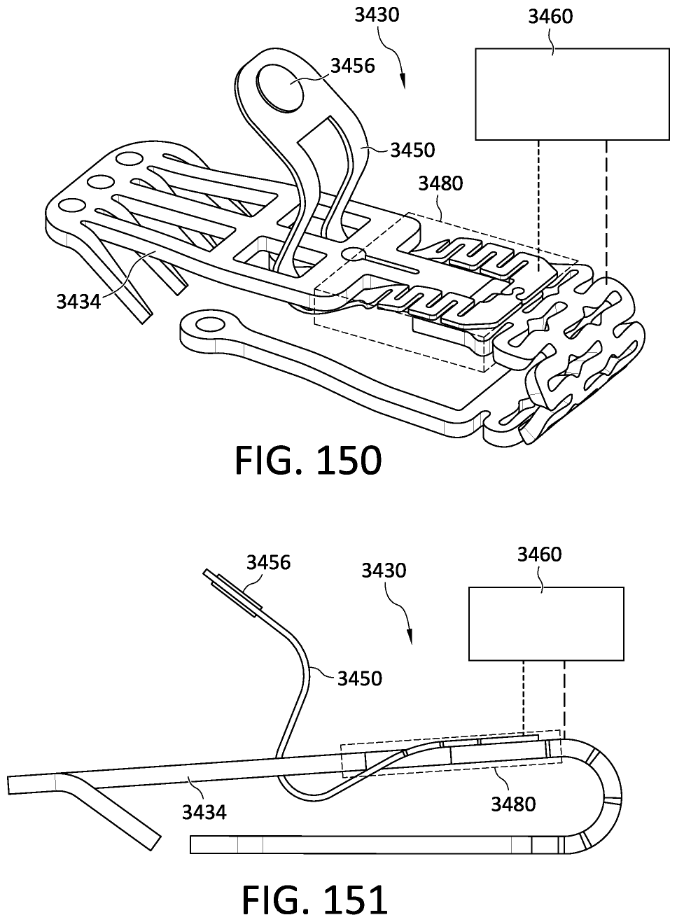

With reference to FIGS. 148-155, by being insulated in region 3480, such as by one or more insulating components, an electrical signal indicating whether or not there is a leaflet disposed within the clasp can be determined by the sensor 3460. When a leaflet does not engage the indicator 3450 within the clasp 3430, the visual indicator 3450 makes electrical contact with the clasp 3430 and the circuit is closed (see FIGS. 148, 149, 152, and 153). When a leaflet engages the indicator 3450 within the clasp 3430, the visual indicator 3450 does not make electrical contact with the clasp 3430 and the circuit is open (see FIGS. 150, 151, 154, and 155).

With reference to FIGS. 148-149, and 152-153, the indicator 3450 is in a non-engaged position, which can be when a leaflet is not positioned within the clasp 3430. In the non-engaged position, indication that there is not a leaflet can be seen visually through the position of the indicator marker 3456, which does not move from the movable arm 3434 of the clasp 3430, as well as through the closed circuit comprising the sensor 3460, the clasp 3430, the indicator 3450, and wiring connecting the clasp 3430 and the indicator 3450 to the sensor 3460. However, in other implementations, the insulator or insulators can be configured such that the circuit is open when the visual indicator is in the non-engaged position. For example, insulators can be positioned at the marker 3456 and at the crossbar of the clasp to insulate the visual indicator from the clasp in the non-engaged position.

With reference to FIGS. 150-151 and 154-155, the indicator 3450 is in an engaged position, which can be when a leaflet is positioned within the clasp 3430. In the engaged position, indication that there is a leaflet can be seen visually through the position of the indicator marker 3456, which has moved a measurable distance from the movable arm 3434 of the clasp 3430, as well as through the open circuit comprising the sensor 3460, the clasp 3430, the indicator 3450, and wiring connecting the clasp 3430 and the indicator 3450 to the sensor 3460. However, in other implementations, the insulator or insulators can be configured such that the circuit is closed when the visual indicator is in the engaged position. For example, the insulators can be configured such that the crossbar of the clasp is not insulated from the curved portion of the visual indicator such that the curved portion of the indicator directly engages the crossbar of the clasp in the engaged position.

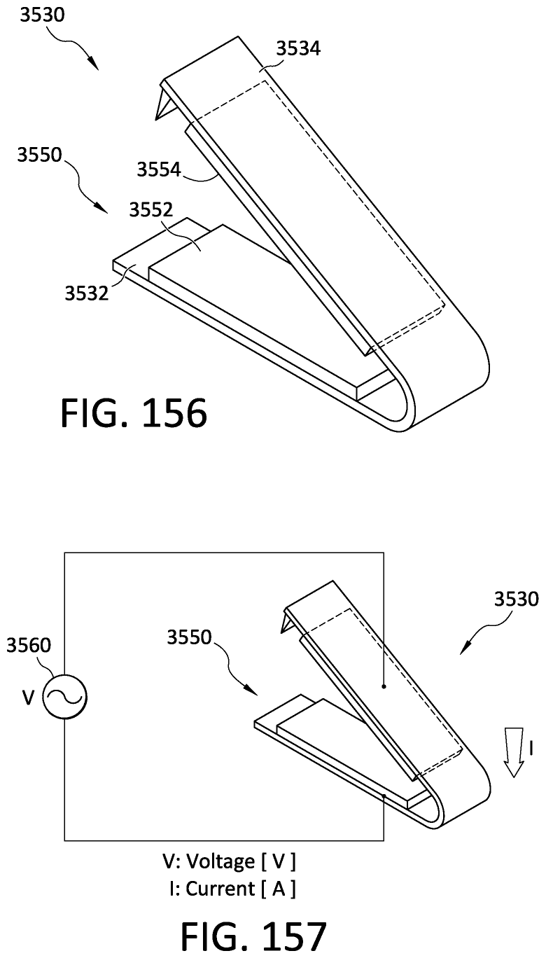

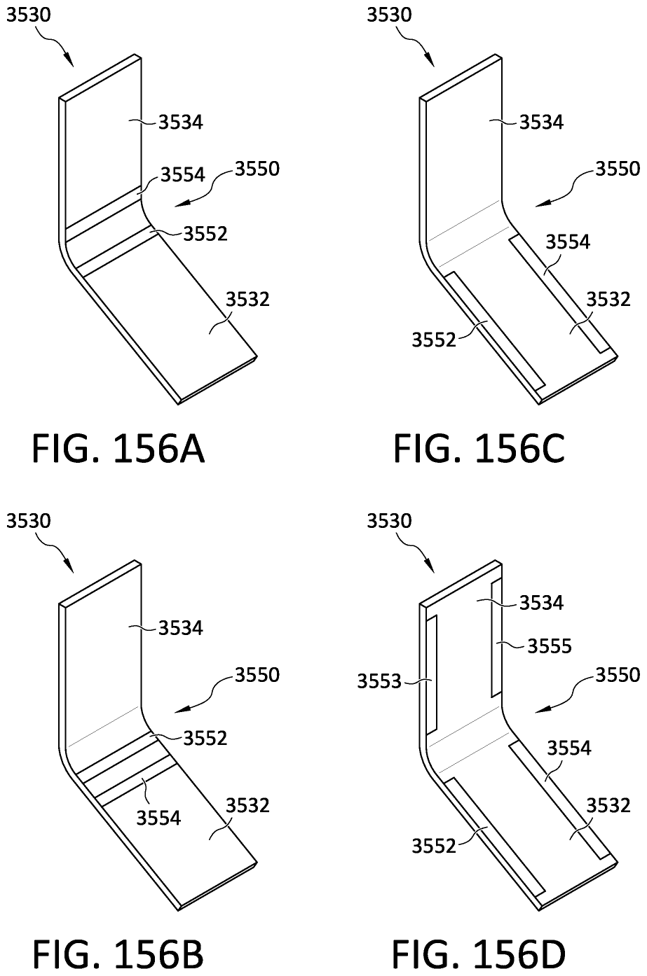

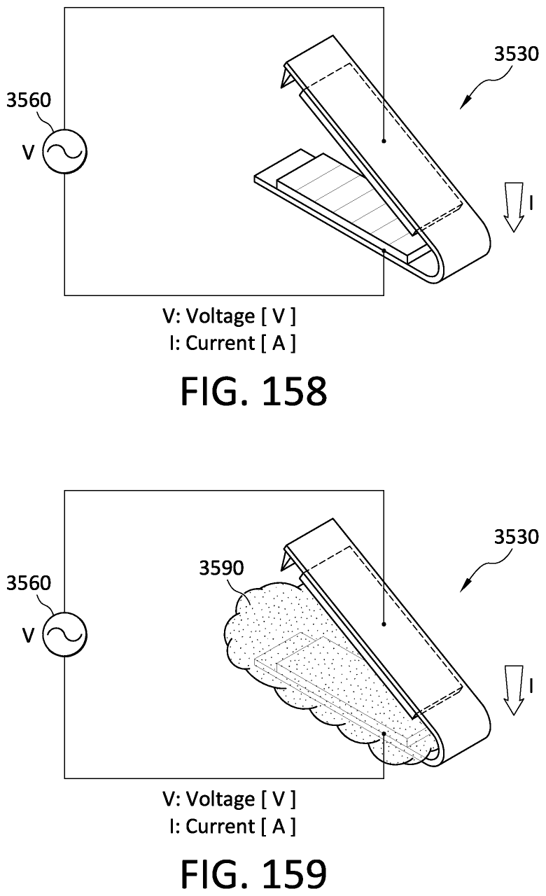

With reference to FIGS. 156-158, implementations of clasps 3530 having electrical indicators 3550 are illustrated. The electrical indicators 3550 can take a variety of different forms. For example, the indicator 3550 can comprise one or more plates. With reference to FIG. 156, an example indicator 3550 comprises a first indicator plate 3552 and a second indicator plate 3554. In accordance with some implementations, the first indicator plate 3552 is coupled to the fixed arm 3532 of the clasp 3530, and the second indicator plate 3554 is coupled to the moveable arm 3534 of the clasp 3530. With reference to FIG. 158, the indicator plates can be made of one or more separate plates. The indicator plates can be made of an electrically conductive material.

FIGS. 156A-156D illustrate additional indicator plate configurations. The implementations illustrated by FIGS. 156, 156A-156D, and 158 are a few examples of the many configurations that can be used. In the implementation illustrated by FIG. 156A, first and second plates 3552, 3554 are positioned near a hinge portion of the clasp 3530. In other implementations, a plate is positioned only on the movable arm 3534 of the clasp or only on the fixed arm of the clasp. The plates 3552, 3554 can be positioned at or near a minimum acceptable leaflet insertion depth.

In the implementation illustrated by FIG. 156B, first and second plates 3552, 3554 are positioned on the fixed arm 3532 of the clasp 3530. In other implementations, the first and second plates 3552, 3554 are positioned on the movable arm 3534 of the clasp. In other implementations, a pair of plates is disposed on the fixed arm 3532 of the clasp and a pair of plates are disposed on the movable arm of the clasp. In the implementation illustrated by FIG. 156B, the indicator plate 3554 can correspond to a minimum leaflet insertion depth and the indicator plate 3552 can correspond to a maximum leaflet insertion depth.

In the implementation illustrated by FIG. 156C, first and second plates 3552, 3554 are positioned on the fixed arm 3532 of the clasp 3530. In other implementations, the first and second plates 3552, 3554 are positioned on the movable arm 3534 of the clasp. in the implementation illustrated by FIG. 156C, the first and second plates 3552, 3554 extend along a length of the clasp arm. The first and second plates 3552, 3554 are spaced apart by a gap. The implementation illustrated by FIG. 156C allows the indicator 3550 detect variations in the presence or depth of tissue, such as leaflet tissue, across the width of the clasp. For example, the configuration illustrated by FIG. 156C can sense that a leaflet is crooked or askew or otherwise improperly grasped by the clasp. The implementation illustrated by FIG. 156D is the same as the implementation illustrated by FIG. 156C, except that a pair of plates is disposed on the fixed clasp arm 3532 and a pair of plates is disposed on the movable clasp arm 3534.

Referring to FIGS. 157 and 158, in some implementations an AC voltage is applied across the electrical indicator(s) and one or more impedance measurements are taken and/or derived. The applied AC voltage can be varied. Different materials can have different impedance characteristics for different applied AC voltages. As such, applying varying AC voltages can allow for enhanced differentiation between different biological materials disposed in the clasp. Any of the electrical indicators disclosed here in can be used with one or more AC voltages applied and one or more impedance measurements taken.

In some implementations, the AC voltage is applied and one or more impedance characteristics are measured while the clasp is closed. In other implementations, the AC voltage is applied and one or more impedance characteristics are measured while the class is open, partially open, or not fully closed. Taking the impedance measurements while the clasp is open, partially open, or not fully closed can have the benefit of being able to confirm that leaflet tissue is properly positioned in the clasp and/or to confirm that another unwanted tissue, such as chordae tendinea is not positioned in the clasp, before the clasp is closed. The clasp can take a variety of different forms. For example, the clasp can be any of the clasps disclosed in the present patent application. The clasps can include optional barbs or other friction-enhancing or securing elements. Taking the impedance measurements while the clasp is open, partially open, or not fully closed can prevent or inhibit the optional barbs from piercing or penetrating the leaflet until it is confirmed that the leaflet is properly positioned in the clasp. Taking the impedance measurements while the clasp is open, partially open, or not fully closed can prevent or inhibit chordae tendinea from being closed in the clasp.

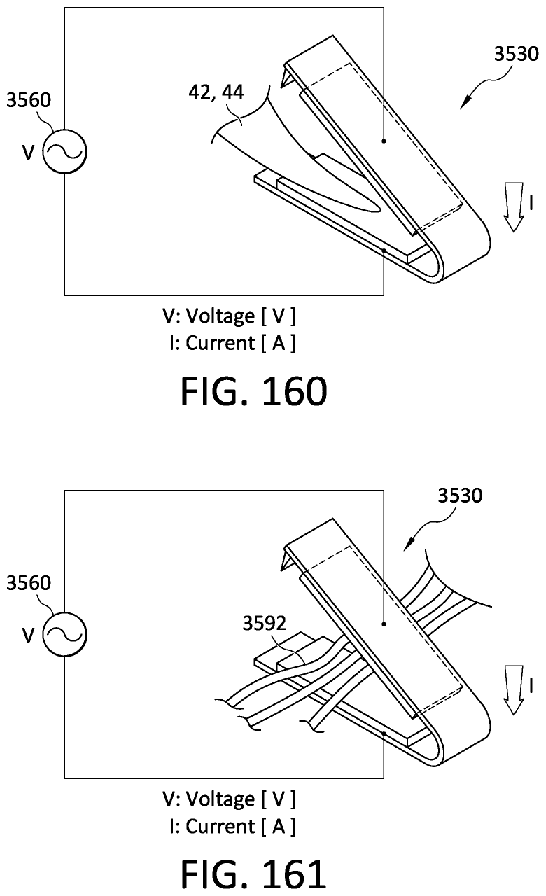

With reference to FIGS. 157-162, the indicator 3550 can be included in a circuit along with the AC power supply, the electrical sensor 3560, and the wiring. The sensor 3560 and the AC power supply can be a single device or separate devices. The wiring connects the first indicator plate 3552 and the second indicator plate 3554 to the AC power supply and the electrical sensor 3560 to measure, among other things, resistance, inductance, capacitance, voltage, current, and/or impedance, components of impedance, etc. The sensor 3560 can measure electrical characteristics in various locations and situations, including when the indicator 3550 is in the presence of blood 3590 (FIG. 159), leaflets 42, 44 (FIG. 160), and chordae tendinea 3592 or other portion of the heart valve instead of the leaflet (FIG. 161). The resistance, inductance, capacitance, voltage, impedance, and/or current readings taken by the sensor can be different based on the anatomy or anatomies that the indicator 3550 is in contact with. Thus, the electrical characteristics measured by the electrical sensor 3560 can be used to determine the location of the clasp and/or the anatomy that the clasp is in contact with, based on the resistance, inductance, capacitance, voltage, impedance and/or current readings taken by the sensor.

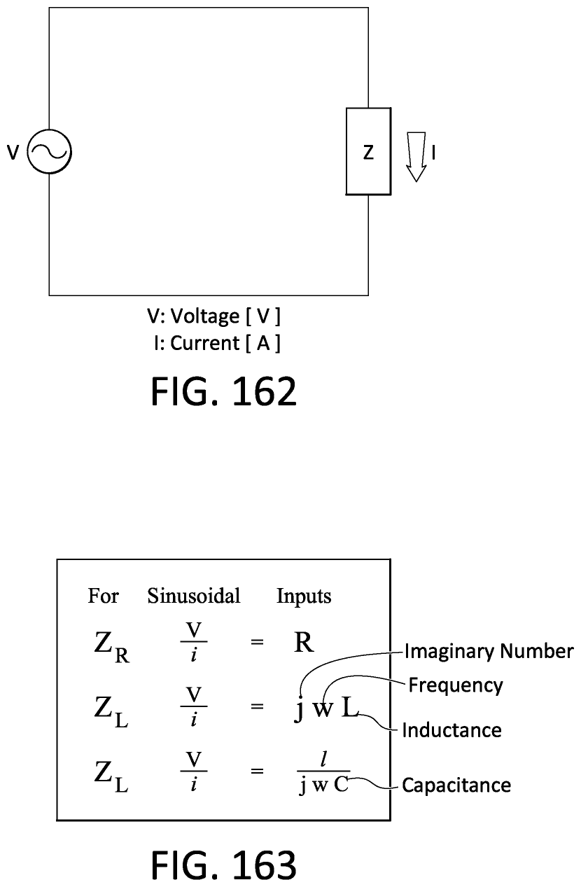

With reference to FIG. 162, the impedance can be measured using a sensor 3560. The sensor can take a variety of different forms, including an impedance meter. Impedance is a quantity that expresses resistance to the flow of an AC current. The magnitude of the impedance Z is equal to the maximum value of the potential difference, or voltage, V (volts) across the circuit, divided by the maximum value of the current I (amperes) through the circuit. Thus, by controlling the AC voltage and measuring the current for any given scenario, the impedance can be calculated.

With reference to FIG. 163, the impedance of an ideal resistor is purely real and is called resistive impedance Z_{R}, and can be measured by dividing voltage (V) by current (I). Ideal inductors and capacitors have a purely imaginary reactive impedance. The impedance of inductors increases as frequency increases, and is calculated as jwL, or the imaginary product of frequency and inductance. The impedance of capacitors decreases as frequency increases, and can be calculated as 1/(jwC) or the imaginary inverse of the product of frequency and capacitance.

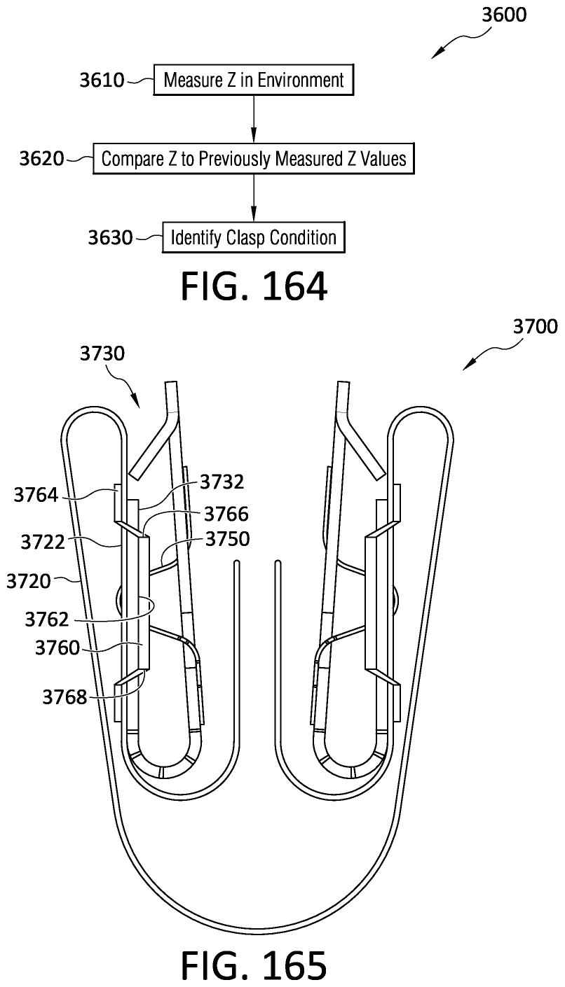

With reference to FIG. 164, a method 3600 of identifying the clasp condition is illustrated. The method 3600 includes the step of measuring 3610 a first impedance value. The impedance can be measured in a variety of different ways. The resistance component R of impedance, the inductance component L of impedance, and/or the capacitance component C of impedance can be measured or derived from the measurements. Impedance can be measured by a sensor used in a circuit according to FIGS. 144-161, and can be measured in accordance with the measurements described in FIGS. 162-163. For example, the impedance between the plates of the indicators 3550 illustrated by FIGS. 156-161 can be measured. In other implementations, impedance between components of any of the indicators disclosed here in can be measured.

The method 3600 also includes the step of comparing 3620 the impedance Z values to a set of previously collected measured values. The previously collected impedance values can correspond to known conditions. For example, each of the previously collected impedance values can be for a type of tissue in a clasp, such as leaflet tissue or chordae tendinea, an amount of tissue in the clasp, a fluid, such as blood in the clasp and/or surrounding the tissue, etc. The previously measured impedance values and associated conditions can be collected, analyzed, and/or processed to predict or estimate conditions associated with future measurements. For example, look-up tables, predictive algorithms, and/or machine learning strategies can be formed using the previously measured impedance values and corresponding conditions. These look up tables, predictive algorithms, and/or machine learning strategies can then be used to identify, estimate, and/or predict a condition that corresponds to a future measured impedance value, such as the impedance value measured in step 3610.

The method 3600 also includes the step of identifying or estimating 3630 the condition and/or location of the clasp. The clasp condition can be determined by comparing the measured impedance value to the condition associated with the corresponding values of the previously measured impedance values. The clasp condition can include a determination of where the clasp is located, what the clasp is attached to, etc. The method 3600 can determine, for example, if the clasp is coupled with a leaflet, and if so, the amount of insertion of said leaflet into the clasp.

When the leaflet is captured by a valve repair device, the leaflet can be pressed between the indicator and the clasp. In some instances, a small or thin leaflet can be at least partially bunched between certain portions of the indicator, or between the indicator and the clasp such that the distance that the indicator is pushed is reduced. With reference to FIGS. 165-169, in some implementations, the device 3700 can include a bar coupled with at least one of the fixed arm 3732 of the clasp 3730 and the inner paddle 3722. The bar can reinforce the inner paddle 3722 and can prevent or inhibit the leaflet from bunching around or between portions of the indicator 3750. As such, when the leaflet is captured within the clasp of the device, the contact between the leaflet and the bar can ensure that the contact between the leaflet and the indicator is sufficient to be identified by a user. The bar can be included in any device disclosed herein, as well as any other valve repair device.

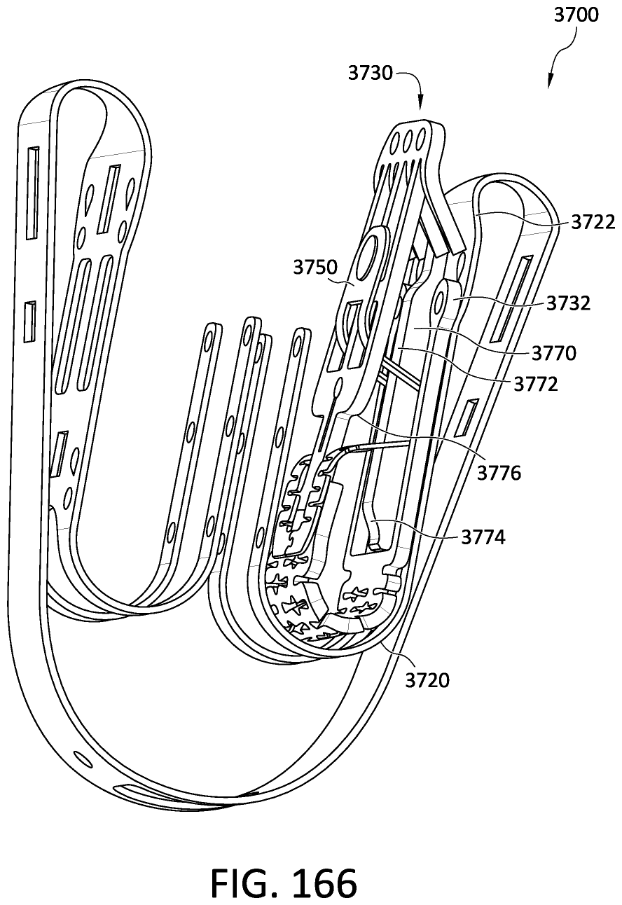

With reference to FIG. 165, the bar 3760 can include a leaflet engaging portion 3762 and a device engaging portion 3764. The bar 3760 can be disposed in a gap between the leaflet engaging portions 3758 of the indicator 3750 (see FIG. 166). The leaflet engaging portion 3762 can have a variety of shapes and sizes. For example, the leaflet engaging portion 3762 of the bar 3760 can make contact with the fixed arm 3732 of the clasp 3730 and/or be flush with a surface of the fixed arm of the clasp 3730 from a first end 3766 of the leaflet engaging portion 3762 to a second end 3768 of the leaflet engaging portion. The bar 3760 can be disposed through or around the fixed arm 3732 of the clasp 3730 and the inner paddle 3722 such that the device engaging portion 3764 is hooked onto or otherwise secured to the inner paddle 3722 at a position between the inner paddle 3722 and the outer paddle 3720. The bar 3760 can assist in further stabilization of the leaflet 42, 44 when the leaflet 42, 44 is engaged in the clasp 3730 by the indicator arm 3750. Specifically, the leaflet 42, 44 is pressed against the two legs of the indicator arm 2050 and the bar 3760, leading to further stabilization of the leaflet 42, 44. The bar 3760 causes an undulating path of the leaflet 42, 44.

The bar can have a variety of different contours. For example, the contours can be selected to optimize or enhance visualization of the indicator 3750 and/or optimize or enhance engagement or grasping of the leaflet by the clasp 3730. In the implementation illustrated by FIG. 166, the bar 3770 can include one or more ridges 3774 on or adjacent to the leaflet engaging portion 3772. The bar 3767 extends substantially into the gap between the leaflet engaging portions 3758 of the indicator 3750. As such, the bar 3770 will increase the movement of the indicator 3750 when the leaflet is disposed in the clasp and/or the leaflet will be grasped more firmly by the closed clasp.

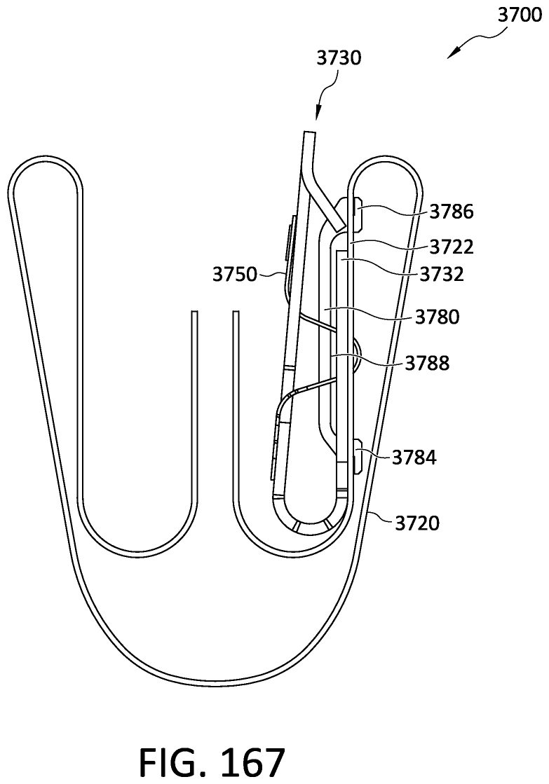

With reference to FIG. 167, a bar 3780 can be positioned such that the surface 3788 does not make contact with the fixed arm 3732 of the clasp 3730 and/or the surface 3788 is spaced apart from the fixed arm 3732 of the clasp 3730. The device engaging portion 3784 can include a clasp region 3786 which can be positioned around the inner paddle 3722 to secure the bar 3780 to the device 3700.

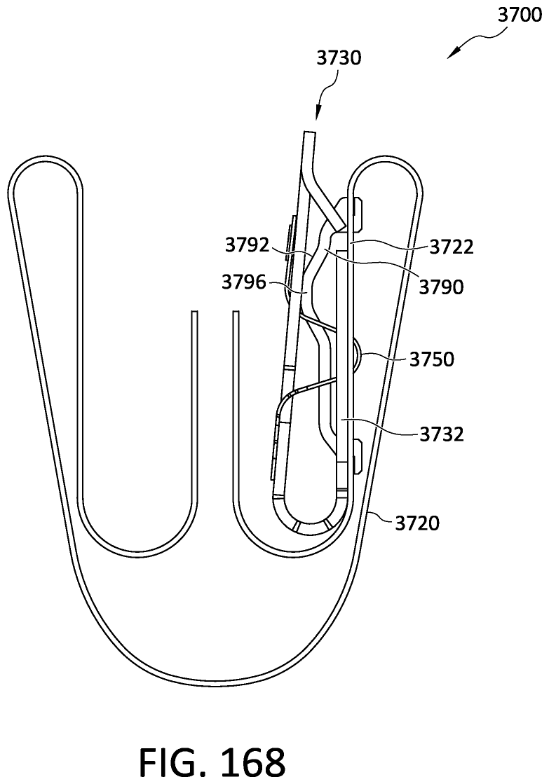

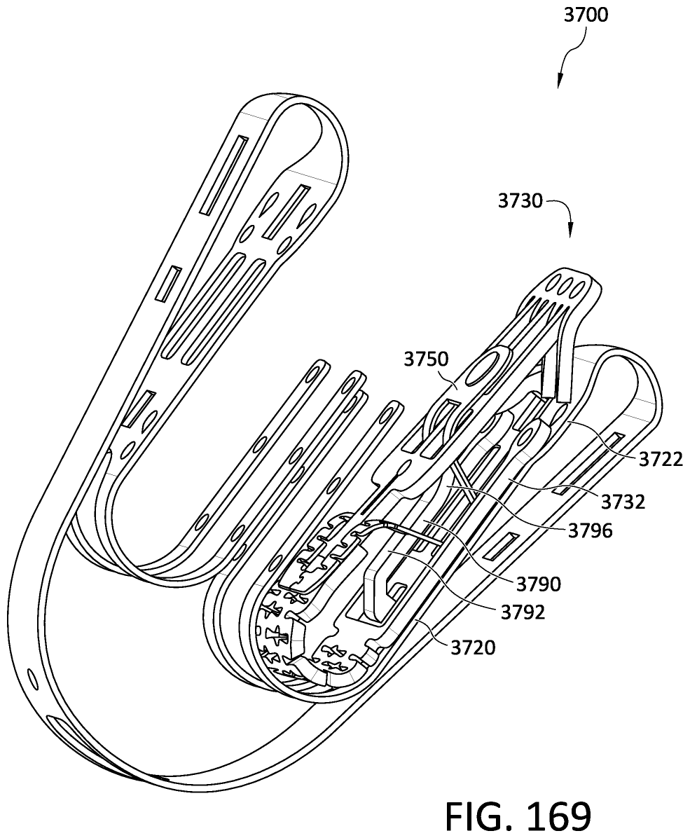

With reference to FIG. 168-169, a leaflet engaging portion 3792 of a bar 3790 can include one or more crests 3796. The crest(s) 3796 of the bar 3790 can be configured to cause the indicator 3750 to move substantially and provide a visual indication as soon as the leaflet has reached a minimum insertion depth. The crest can be configured to cause the indicator 3750 to move substantially as soon as the leaflet has reached the minimum insertion depth in a variety of different ways. In the illustrated example, the crest 3796 abuts the movable arm 3734 and/or is very close to the movable arm 3734. Also, when viewed from the side, as in FIG. 168, the profile of the crest 3796 overlaps the leaflet engaging portions of the indicator 3750. As a result, the indicator 3750 will move substantially as soon as the leaflet has reached the overlap between the crest 3796 and the leaflet engaging portions of the indicator 3750. In some implementations, the overlap is selected to coincide with the minimum leaflet insertion depth.

In some implementations, the crest 3796 can be configured to cause the clasp 3730 to engage portion of the leaflet more firmly in the proximal end (toward the open end) of the clasp 3730 than the portion of the leaflet in the distal end (toward the closed end) of the clasp. The crest 3796 can be configured to cause the clasp 3730 to engage the portion of the leaflet more firmly in the proximal end of the clasp 3730 than the portion of the leaflet in the distal end of the clasp in a variety of different ways. In the implementation illustrated by FIGS. 168 and 169, a crest 3796 is included near the proximal end of the clasp, but no crest is included at the distal end of the clasp. As such, a portion of the leaflet closer to the proximal end of the clasp is engaged more firmly than a portion of the leaflet near a distal end of a clasp. In other implementations, crests 3796 can be included at multiple locations, such as at both the proximal end of the clasp and at the distal end of the clasp.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The various inventive aspects, concepts and features of the disclosures may be described and illustrated herein as embodied in combination in the examples herein, these various aspects, concepts, and features may be used in many alternative implementations, either individually or in various combinations and sub-combinations thereof. Unless expressly excluded herein all such combinations and sub-combinations are intended to be within the scope of the present application. Still further, while various alternative implementations as to the various aspects, concepts, and features of the disclosures—such as alternative materials, structures, configurations, methods, devices, and components, alternatives as to form, fit, and function, and so on—may be described herein, such descriptions are not intended to be a complete or exhaustive list of available implementations, whether presently known or later developed. Those skilled in the art may readily adopt one or more of the inventive aspects, concepts, or features into additional implementations and uses within the scope of the present application even if such implementations are not expressly disclosed herein.

Additionally, even though some features, concepts, or aspects of the disclosures may be described herein as being a preferred arrangement or method, such description is not intended to suggest that such feature is required or necessary unless expressly so stated. Still further, example or representative values and ranges may be included to assist in understanding the present application, however, such values and ranges are not to be construed in a limiting sense and are intended to be critical values or ranges only if so expressly stated.

Moreover, while various aspects, features and concepts may be expressly identified herein as being inventive or forming part of a disclosure, such identification is not intended to be exclusive, but rather there may be inventive aspects, concepts, and features that are fully described herein without being expressly identified as such or as part of a specific disclosure, the disclosures instead being set forth in the appended claims. Descriptions of example methods or processes are not limited to inclusion of all steps as being required in all cases, nor is the order that the steps are presented to be construed as required or necessary unless expressly so stated. Further, the techniques, methods, operations, steps, etc. described or suggested herein can be performed on a living animal or on a non-living simulation, such as on a cadaver, cadaver heart, simulator (e.g., with the body parts, tissue, etc. being simulated), etc. The words used in the claims have their full ordinary meanings and are not limited in any way by the description of the implementations in the specification.

What is claimed is:

1. A valve repair device for repairing a native valve of a patient, the valve repair device comprising:
a central component;
a paddle coupled to the central component;
a gripping member;
wherein the gripping member and the paddle are movable to form a capture region for capturing a leaflet of the native valve;
an indicator that is configured to move into the capture region when the gripping member is in a closed position, the paddle is in a closed position and the leaflet of the native valve is not disposed in the capture region; and
wherein the indicator is configured to push against the central component and be pressed to a flattened condition when the gripping member is in the closed position, the paddle is in the closed position and the leaflet of the native valve is disposed in the capture region at least a minimum insertion depth.

2. The valve repair device of claim 1 wherein the indicator is configured to pass through one or more of the paddle and the gripping member.

3. The valve repair device of claim 1 wherein the paddle includes an inner paddle portion and an outer paddle portion, wherein at least a portion of the indicator is disposed between the inner paddle portion and the outer paddle portion when the gripping member is in the closed position, the paddle is in the closed position and the leaflet of the native valve is disposed in the capture region at the minimum insertion depth.

4. The valve repair device of claim 1, wherein the gripping member and the indicator each comprise a marker comprising a radiopaque material.

5. The valve repair device of claim 1, wherein the central component comprises a spacer.

6. The valve repair device of claim 1, wherein a fixed arm of the gripping member comprises a first beam, a second beam, and an engaging member between the first beam and the second beam.

7. The valve repair device of claim 6, further comprising an indicator marker attached to the indicator.

8. The valve repair device of claim 1, wherein the indicator is configured as an indicator arm comprises a fixed end and a moving end.

9. The valve repair device of claim 1, wherein the indicator is formed from a portion of the gripping member, wherein the indicator is formed between outer beams of a movable arm of the gripping member or outside the outer beams of the gripping member, wherein the indicator comprises a twisted portion, and wherein the twisted portion comprises one or more twists between 0 degrees and 180 degrees.

10. A valve repair system for repairing a native valve of a patient, the valve repair system comprising:
a delivery system;
a valve repair device coupled to the delivery system, the valve repair device comprising:
a central component;
a paddle coupled to the central component;
a gripping member;
wherein the gripping member and the paddle are movable to form a capture region for capturing a leaflet of the native valve;
an indicator that is configured to move into the capture region when the gripping member is in a closed position, the paddle is in a closed position and the leaflet of the native valve is not disposed in the capture region; and
wherein the indicator is configured to push against the central component and be pressed to a flattened condition when the gripping member is in the closed position, the paddle is in the closed position and the leaflet of the native valve is disposed in the capture region at least a minimum insertion depth.

11. The valve repair system of claim 10 wherein the indicator is configured to pass through one or more of the paddle and the gripping member.

12. The valve repair system of claim 10 wherein the paddle includes an inner paddle portion and an outer paddle portion, wherein at least a portion of the indicator is disposed between the inner paddle portion and the outer paddle portion when the gripping member is in the closed position, the paddle is in the closed position and the leaflet of the native valve is disposed in the capture region at the minimum insertion depth.

13. The valve repair system of claim 10, wherein the gripping member and the indicator each comprise a marker comprising a radiopaque material.

14. The valve repair system of claim 10, wherein the gripping member and the indicator are made from a single piece of flat material.

15. The valve repair system of claim 10, wherein a fixed arm of the gripping member comprises a first beam, a second beam, and an engaging member between the first beam and the second beam.

16. The valve repair system of claim 15, further comprising an indicator marker attached to the indicator.

17. The valve repair system of claim 10, wherein the indicator is configured as an indicator arm comprises a fixed end and a moving end.

18. The valve repair system of claim 10, wherein the indicator is formed from a portion of the gripping member, wherein the indicator is formed between outer beams of a movable arm of the gripping member or outside the outer beams of the gripping member, wherein the indicator comprises a twisted portion, and wherein the twisted portion comprises one or more twists between 0 degrees and 180 degrees.

19. A valve repair device for repairing a native valve of a patient, the valve repair device comprising:
a gripper arm;
wherein the gripper arm are movable to form an opening or capture region for receiving a native leaflet of the native valve; and
a leaflet depth indicator comprising a first electrode and a second electrode, wherein the first electrode and the second electrode provide electrical signals to indicate whether a leaflet of the native valve is inserted into the opening to a particular insertion depth.

20. The valve repair device of claim 19, wherein the electrical signals comprises an intracardiac electrocardiogram signal or a bioimpedance signal.
